SEQUENCE LISTING (306 sequences)

SEQ ID NO: 1 - protein, 450 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSQPLNPGKCACECTESPQKCLLKGKKFHHQTCSCYRRPCTNRQKACEPGFS

SEQ ID NO: 2 - DNA, 1460 bp, Homo sapiens
  source 1..1460: mol_type = genomic DNA
actggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaa

SEQ ID NO: 3 - DNA, 1248 bp, Mus sp.
  source 1..1248: mol_type = genomic DNA
atgcacttgctgtgcttcttgtctctggcgtgttccctgctcgccgctgcgctgatccccagtccgcgcgaggcgcccgccaccgtcgccgccttcgagtcgggactgggcttctcggaagcggagcccgacgggggcgaggtcaaggcttttgaaggcaaagacctggaggagcagttgcggtctgtgtccagcgtagatgagctgatgtctgtcctgtacccagactactggaaaatgtacaagtgccagctgcggaaaggcggctggcagcagcccaccctcaataccaggacaggggacagtgtaaaatttgctgctgcacattataacacagagatcctgaaaagtattgataatgagtggagaaagactcaatgcatgccacgtgaggtgtgtatagatgtggggaaggagtttggagcagccacaaacaccttctttaaacctccatgtgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgttctctgccagcaacattaccacagtgtcaggcagctaacaagacatgtccaacaaactatgtgtggaataactacatgtgccgatgcctggctcagcaggattttatcttttattcaaatgttgaagatgactcaaccaatggattccatgatgtctgtggacccaacaaggagctggatgaagacacctgtcagtgtgtctgcaagggggggcttcggccatctagttgtggaccccacaaagaactagatagagactcatgtcagtgtgtctgtaaaaacaaacttttccctaattcatgtggagccaacagggaatttgatgagaatacatgtcagtgtgtatgtaaaagaacgtgtccaagaaatcagcccctgaatcctgggaaatgtgcctgtgaatgtacagaaaacacacagaagtgcttccttaaagggaagaagttccaccatcaaacatgcagttgttacagaagaccgtgtgcgaatcgactgaagcattgtgatccaggactgtcctttagtgaagaagtatgccgctgtgtcccatcgtattggaaaaggccacatctgaactaa

SEQ ID NO: 4 - protein, 415 aa, Mus sp.
MHLLCFLSLACSLLAAALIPSPREAPATVAAFESGLGFSEAEPDGGEVKAFEGKDLEEQLRSVSSVDELMSVLYPDYWKMYKCQLRKGGWQQPTLNTRTGDSVKFAAAHYNTEILKSIDNEWRKTQCMPREVCIDVGKEFGAATNTFFKPPCVSVYRCGGCCNSEGLQCMNTSTGYLSKTLFEITVPLSQGPKPVTISFANHTSCRCMSKLDVYRQVHSIIRRSLPATLPQCQAANKTCPTNYVWNNYMCRCLAQQDFIFYSNVEDDSTNGFHDVCGPNKELDEDTCQCVCKGGLRPSSCGPHKELDRDSCQCVCKNKLFPNSCGANREFDENTCQCVCKRTCPRNQPLNPGKCACECTENTQKCFLKGKKFHHQTCSCYRRPCANRLKHCDPGLSFSEEVCRCVPSYWKRPHLN

SEQ ID NO: 5 - DNA, 1248 bp, synthetic construct
  source 1..1248: mol_type = other DNA
atgcacttgctgtgcttcttgtctctggcgtgttccctgctcgccgctgcgctgatccccagtccgcgcgaggcgcccgccaccgtcgccgccttcgagtcgggactgggcttctcggaagcggagcccgacgggggcgaggtcaaggcttttgaaggcaaagacctggaggagcagttgcggtctgtgtccagcgtagatgagctgatgtctgtcctgtacccagactactggaaaatgtacaagtgccagctgcggaaaggcggctggcagcagcccaccctcaataccaggacaggggacagtgtaaaatttgctgctgcacattataacacagagatcctgaaaagtattgataatgagtggagaaagactcaatgcatgccacgtgaggtgtgtatagatgtggggaaggagtttggagcagccacaaacaccttctttaaacctccatgtgtgtccgtctacagatgtgggggttgctgcatcagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgttctctgccagcaacattaccacagtgtcaggcagctaacaagacatgtccaacaaactatgtgtggaataactacatgtgccgatgcctggctcagcaggattttatcttttattcaaatgttgaagatgactcaaccaatggattccatgatgtctgtggacccaacaaggagctggatgaagacacctgtcagtgtgtctgcaagggggggcttcggccatctagttgtggaccccacaaagaactagatagagactcatgtcagtgtgtctgtaaaaacaaacttttccctaattcatgtggagccaacagggaatttgatgagaatacatgtcagtgtgtatgtaaaagaacgtgtccaagaaatcagcccctgaatcctgggaaatgtgcctgtgaatgtacagaaaacacacagaagtgcttccttaaagggaagaagttccaccatcaaacatgcagttgttacagaagaccgtgtgcgaatcgactgaagcattgtgatccaggactgtcctttagtgaagaagtatgccgctgtgtcccatcgtattggaaaaggccacatctgaactaa

SEQ ID NO: 6 - protein, 415 aa, synthetic construct
MHLLCFLSLACSLLAAALIPSPREAPATVAAFESGLGFSEAEPDGGEVKAFEGKDLEEQLRSVSSVDELMSVLYPDYWKMYKCQLRKGGWQQPTLNTRTGDSVKFAAAHYNTEILKSIDNEWRKTQCMPREVCIDVGKEFGAATNTFFKPPCVSVYRCGGCCISEGLQCMNTSTGYLSKTLFEITVPLSQGPKPVTISFANHTSCRCMSKLDVYRQVHSIIRRSLPATLPQCQAANKTCPTNYVWNNYMCRCLAQQDFIFYSNVEDDSTNGFHDVCGPNKELDEDTCQCVCKGGLRPSSCGPHKELDRDSCQCVCKNKLFPNSCGANREFDENTCQCVCKRTCPRNQPLNPGKCACECTENTQKCFLKGKKFHHQTCSCYRRPCANRLKHCDPGLSFSEEVCRCVPSYWKRPHLN

SEQ ID NO: 7 - DNA, 1248 bp, synthetic construct
  source 1..1248: mol_type = other DNA
atgcacttgctgtgcttcttgtctctggcgtgttccctgctcgccgctgcgctgatccccagtccgcgcgaggcgcccgccaccgtcgccgccttcgagtcgggactgggcttctcggaagcggagcccgacgggggcgaggtcaaggcttttgaaggcaaagacctggaggagcagttgcggtctgtgtccagcgtagatgagctgatgtctgtcctgtacccagactactggaaaatgtacaagtgccagctgcggaaaggcggctggcagcagcccaccctcaataccaggacaggggacagtgtaaaatttgctgctgcacattataacacagagatcctgaaaagtattgataatgagtggagaaagactcaatgcatgccacgtgaggtgtgtatagatgtggggaaggagtttggagcagccacaaacaccttctttaaacctccatgtgtgtccgtctacagatgtgggggttgctgcagaagcgaggggctgcagtgcatgaacaccagcacaggttacctcagcaagacgttgtttgaaattacaactcctatttcacaaggccccaaaccagtcacaatcagttttgccaatcacacttcctgccggtgcatgtctaaactggatgtttacagacaagttcattcaattattagacgttctctgccagcaacattaccacagtgtcaggcagctaacaagacatgtccaacaaactatgtgtggaataactacatgtgccgatgcctggctcagcaggattttatcttttattcaaatgttgaagatgactcaaccaatggattccatgatgtctgtggacccaacaaggagctggatgaagacacctgtcagtgtgtctgcaagggggggcttcggccatctagttgtggaccccacaaagaactagatagagactcatgtcagtgtgtctgtaaaaacaaacttttccctaattcatgtggagccaacagggaatttgatgagaatacatgtcagtgtgtatgtaaaagaacgtgtccaagaaatcagcccctgaatcctgggaaatgtgcctgtgaatgtacagaaaacacacagaagtgcttccttaaagggaagaagttccaccatcaaacatgcagttgttacagaagaccgtgtgcgaatcgactgaagcattgtgatccaggactgtcctttagtgaagaagtatgccgctgtgtcccatcgtattggaaaaggccacatctgaactaa

SEQ ID NO: 8 - protein, 415 aa, synthetic construct
MHLLCFLSLACSLLAAALIPSPREAPATVAAFESGLGFSEAEPDGGEVKAFEGKDLEEQLRSVSSVDELMSVLYPDYWKMYKCQLRKGGWQQPTLNTRTGDSVKFAAAHYNTEILKSIDNEWRKTQCMPREVCIDVGKEFGAATNTFFKPPCVSVYRCGGCCRSEGLQCMNTSTGYLSKTLFEITTPISQGPKPVTISFANHTSCRCMSKLDVYRQVHSIIRRSLPATLPQCQAANKTCPTNYVWNNYMCRCLAQQDFIFYSNVEDDSTNGFHDVCGPNKELDEDTCQCVCKGGLRPSSCGPHKELDRDSCQCVCKNKLFPNSCGANREFDENTCQCVCKRTCPRNQPLNPGKCACECTENTQKCFLKGKKFHHQTCSCYRRPCANRLKHCDPGLSFSEEVCRCVPSYWKRPHLN

SEQ ID NO: 9 - protein, 101 aa, synthetic construct
NTEILKSIDNEWRKTQCMPREVAIDVGKEFGAATNTFFKPPCVSVYRCGGCCNSEGLQCMNTSTGYLSKTLFEITVPLSQGPKPVTISFANHTSCRCMSKL

SEQ ID NO: 10 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgcggcaacgaacacattcttcaaacccccttgtgtgagtgtatatagatgtggagggtgttgcaattctgaggggctccaatgtatgaacacgagtacgggttacttgagtaagaccttgttcgaaattacagtaccactttcccaaggaccgaagcctgtgactatcagctttgctaaccatacaagctgtcgctgtatgagcaaactg

SEQ ID NO: 11 - protein, 336 aa, synthetic construct
NTEILKSIDNEWRKTQCMPREVAIDVGKEFGAATNTFFKPPCVSVYRCGGCCNSEGLQCMNTSTGYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1008 bp, synthetic construct
  source 1..1008: mol_type = other DNA
aacacggaaatcctcaagtctatagataatgagtggcgaaagacacaatgtatgccgcgcgaggtagcaatcgacgttggtaaagaatttggtgcggcaacgaacacattcttcaaacccccttgtgtgagtgtatatagatgtggagggtgttgcaattctgaggggctccaatgtatgaacacgagtacgggttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaag

SEQ ID NO: 13 - protein, 101 aa, synthetic construct
NTEILKSIDNEWRKTQCMPREVAIDVGKEFGAATNTFFKPPCVSVYRCGGCCRSEGLQCMNTSTGYLSKTLFEITTPISQGPKPVTISFANHTSCRCMSKL

SEQ ID NO: 14 - DNA, 303 bp, synthetic construct
  source 1..303: mol_type = other DNA
aacaccgaaatcttgaaatcaatagataacgaatggcgaaaaacccaatgtatgccacgcgaggtagcgatagatgtgggcaaagaattcggcgccgcgacgaacacctttttcaagcccccttgcgtctccgtatatagatgcggtggatgttgccgatccgagggccttcagtgtatgaacacatctactggctatttgagcaagacgctctttgagattacaacaccaattagtcaaggtcccaagcctgttaccatctctttcgctaaccacacttcatgccgctgtatgagtaagttg

SEQ ID NO: 15 - protein, 336 aa, synthetic construct
NTEILKSIDNEWRKTQCMPREVAIDVGKEFGAATNTFFKPPCVSVYRCGGCCRSEGLQCMNTSTGYLSKTLFEITTPISQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1008 bp, synthetic construct
  source 1..1008: mol_type = other DNA
aacaccgaaatcttgaaatcaatagataacgaatggcgaaaaacccaatgtatgccacgcgaggtagcgatagatgtgggcaaagaattcggcgccgcgacgaacacctttttcaagcccccttgcgtctccgtatatagatgcggtggatgttgccgatccgagggccttcagtgtatgaacacatctactggctatttgagcaagacgctctttgagattacaacaccaattagtcaaggtcccaagcctgttaccatctctttcgctaaccacacttcatgccgctgtatgagtaagttggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaag

SEQ ID NO: 17 - protein, 101 aa, synthetic construct
NTEILKSIDNEWRKTQCMPREVAIDVGKEFGAATNTFFKPPCVSVYRCGGCCISEGLQCMNTSTGYLSKTLFEITVPLSQGPKPVTISFANHTSCRCMSKL

SEQ ID NO: 18 - DNA, 303 bp, synthetic construct
  source 1..303: mol_type = other DNA
aacaccgaaatcttgaaatcaatagataacgaatggcgcaaaactcaatgtatgccacgggaggttgcaatagacgtgggaaaggaatttggcgccgccacgaatacctttttcaagcctccctgtgtgagtgtttacagatgtggtggttgttgcatatcagagggattgcagtgcatgaacacaagtacaggttacttgagtaaaacattgtttgaaatcacagtaccattgtcccaaggccctaagccggttacgatctctttcgccaatcatacgtcatgccgctgtatgagtaagttg

SEQ ID NO: 19 - protein, 336 aa, synthetic construct
NTEILKSIDNEWRKTQCMPREVAIDVGKEFGAATNTFFKPPCVSVYRCGGCCISEGLQCMNTSTGYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1008 bp, synthetic construct
  source 1..1008: mol_type = other DNA
aacaccgaaatcttgaaatcaatagataacgaatggcgcaaaactcaatgtatgccacgggaggttgcaatagacgtgggaaaggaatttggcgccgccacgaatacctttttcaagcctccctgtgtgagtgtttacagatgtggtggttgttgcatatcagagggattgcagtgcatgaacacaagtacaggttacttgagtaaaacattgtttgaaatcacagtaccattgtcccaaggccctaagccggttacgatctctttcgccaatcatacgtcatgccgctgtatgagtaagttggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaag

SEQ ID NO: 21 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 753 bp, synthetic construct
  source 1..753: mol_type = other DNA
actatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaag

SEQ ID NO: 23 - protein, 101 aa, Mus sp.
NTEILKSIDNEWRKTQCMPREVCIDVGKEFGAATNTFFKPPCVSVYRCGGCCNSEGLQCMNTSTGYLSKTLFEITVPLSQGPKPVTISFANHTSCRCMSKL

SEQ ID NO: 24 - DNA, 303 bp, Mus sp.
  source 1..303: mol_type = genomic DNA
aacacggaaatcctcaagtctatagataatgagtggcgaaagacacaatgtatgccgcgcgaggtatgtatcgacgttggtaaagaatttggtgcggcaacgaacacattcttcaaacccccttgtgtgagtgtatatagatgtggagggtgttgcaattctgaggggctccaatgtatgaacacgagtacgggttacttgagtaagaccttgttcgaaattacagtaccactttcccaaggaccgaagcctgtgactatcagctttgctaaccatacaagctgtcgctgtatgagcaaactg

SEQ ID NO: 25 - protein, 6 aa, synthetic construct
HHHHHH

SEQ ID NO: 26 - protein, 33 aa, synthetic construct
RMKQLEDKIEELLSKIYHLENEIARLKKLIGER

SEQ ID NO: 27 - protein, 4 aa, synthetic construct
STAG

SEQ ID NO: 28 - protein, 9 aa, synthetic construct
LQRIKRVEG

SEQ ID NO: 29 - protein, 9 aa, synthetic construct
DGVINSLHR

SEQ ID NO: 30 - protein, 6 aa, synthetic construct
QHNKDE

SEQ ID NO: 31 - protein, 6 aa, synthetic construct
TLPIVA

SEQ ID NO: 32 - protein, 8 aa, synthetic construct
NITSVADG

SEQ ID NO: 33 - protein, 8 aa, synthetic construct
KRTPQAEG

SEQ ID NO: 34 - protein, 9 aa, synthetic construct
NHDQKERSG

SEQ ID NO: 35 - protein, 8 aa, synthetic construct
EAGTKRPQ

SEQ ID NO: 36 - protein, 6 aa, synthetic construct
ILVPTA

SEQ ID NO: 37 - protein, 6 aa, synthetic construct
VILPTA

SEQ ID NO: 38 - protein, 8 aa, synthetic construct
FLIVYHND

SEQ ID NO: 39 - protein, 9 aa, synthetic construct
PQRTKRAEG

SEQ ID NO: 40 - DNA, 38 bp, synthetic construct
  source 1..38: mol_type = other DNA
gcggttctctggaagttctgttccagggtccgggatcc

SEQ ID NO: 41 - DNA, 54 bp, synthetic construct
  source 1..54: mol_type = other DNA
ggactgcagtgcatgaacaccagcacaggttacctcagcaagacgttgtttgaa

SEQ ID NO: 42 - DNA, 48 bp, synthetic construct
  source 1..48: mol_type = other DNA
aaaccagtcacaatcagttttgccaatcacacttcctgccggtgcatg

SEQ ID NO: 43 - DNA, 48 bp, synthetic construct
  source 1..48: mol_type = other DNA
cagatgatctgtaaacatccagtttagacatgcaccggcaggaagtgt

SEQ ID NO: 44 - DNA, 46 bp, synthetic construct
  source 1..46: mol_type = other DNA
ggatgtttacagatcatctggatctagcggatctgcggccgcttct

SEQ ID NO: 45 - DNA, 44 bp, synthetic construct
  source 1..44: mol_type = other DNA
tcggagataagcttttgttcgccaccagaagcggccgcagatcc

SEQ ID NO: 46 - DNA, 79 bp, synthetic construct
  source 1..79: mol_type = other DNA
cctcacgtggcatgcawtbagtctttctccactcattahbaatacttttthbgatctcasyggatcccggaccctggaa

SEQ ID NO: 47 - DNA, 85 bp, synthetic construct
  source 1..85: mol_type = other DNA
  misc_difference 56: note = a, c, t, g, unknown, or other
tgcatgccacgtgaggtgtgtatagatgtggggaaggagtttggagcagccvytrnaaccttctttvvacctccatgtgtgtccg

SEQ ID NO: 48 - DNA, 70 bp, synthetic construct
  source 1..70: mol_type = other DNA
tggtgttcatgcactgcagtcctbbkybkybgcagcaacctccacatctgtagacggacacacatggagg

SEQ ID NO: 49 - DNA, 68 bp, synthetic construct
  source 1..68: mol_type = other DNA
  misc_difference 36: note = a, c, t, g, unknown, or other
aaaactgattgtgactggttttbbgccttgtgaawnaggarbtgtarbttcaaacaacgtcttgctga

SEQ ID NO: 50 - protein, 101 aa, synthetic construct
NTEILKSIDNEWRKTQCMPREVAIDVGKEFGVATNTFFKPPCVSVYRCGGCCRSEGLQCMNTSTSYLSKTLFEITVPLSQGPKPVTISFANHTSCRCMSKL

SEQ ID NO: 51 - protein, 101 aa, synthetic construct
NTEILKSIDNEWRKTQCMPREVAIDVGKEFGVATNTFFKPPCVSVYRCGGCCRGEGLQCMNTSTSYLSKTLFEITVPLSQGPKPVTISFANHTSCRCMSKL

SEQ ID NO: 52 - protein, 101 aa, synthetic construct
NTEILKSIDNEWRKTQCMPREVAIDVGKEFGVATNTFFKPPCVSVYRCGGCCRSEGLQCMNTSTSYLSKTLFEITVPISQGPKPVTISFANHTSCRCMSKL

SEQ ID NO: 53 - protein, 101 aa, synthetic construct
NTEILKSIDNEWRKTQCMPREVAIDVGKEFGVATNTFFKPPCVSVYRCGGCCRGEGLQCMNTSTSYLSKTLFEITVPISQGPKPVTISFANHTSCRCMSKL

SEQ ID NO: 54 - protein, 101 aa, synthetic construct
NTEILKSIDNEWRKTQCMPREVAIDVGKEFGVATNTFFKPPCVSVYRCGGCCRSEGLQCMNTSTSYLSKTLFEITTPISQGPKPVTISFANHTSCRCMSKL

SEQ ID NO: 55 - protein, 101 aa, synthetic construct
NTEILKSIDNEWRKTQCMPREVAIDVGKEFGVATNTFFKPPCVSVYRCGGCCRGEGLQCMNTSTSYLSKTLFEITTPISQGPKPVTISFANHTSCRCMSKL

SEQ ID NO: 56 - protein, 101 aa, synthetic construct
NTEILKSIDNEWRKTQCMPREVAIDVGKEFGVATNTFFKPPCVSVYRCGGCCQSEGLQCMNTSTSYLSKTLFEITVPLSQGPKPVTISFANHTSCRCMSKL

SEQ ID NO: 57 - protein, 101 aa, synthetic construct
NTEILKSIDNEWRKTQCMPREVAIDVGKEFGVATNTFFKPPCVSVYRCGGCCQGEGLQCMNTSTSYLSKTLFEITVPLSQGPKPVTISFANHTSCRCMSKL

SEQ ID NO: 58 - protein, 101 aa, synthetic construct
NTEILKSIDNEWRKTQCMPREVAIDVGKEFGVATNTFFKPPCVSVYRCGGCCQSEGLQCMNTSTSYLSKTLFEITVPISQGPKPVTISFANHTSCRCMSKL

SEQ ID NO: 59 - protein, 101 aa, synthetic construct
NTEILKSIDNEWRKTQCMPREVAIDVGKEFGVATNTFFKPPCVSVYRCGGCCQGEGLQCMNTSTSYLSKTLFEITVPISQGPKPVTISFANHTSCRCMSKL

SEQ ID NO: 60 - protein, 101 aa, synthetic construct
NTEILKSIDNEWRKTQCMPREVAIDVGKEFGVATNTFFKPPCVSVYRCGGCCQSEGLQCMNTSTSYLSKTLFEITTPISQGPKPVTISFANHTSCRCMSKL

SEQ ID NO: 61 - protein, 101 aa, synthetic construct
NTEILKSIDNEWRKTQCMPREVAIDVGKEFGVATNTFFKPPCVSVYRCGGCCQGEGLQCMNTSTSYLSKTLFEITTPISQGPKPVTISFANHTSCRCMSKL

SEQ ID NO: 62 - protein, 101 aa, synthetic construct
NTEILKSIDNEWRKTQCMPREVAIDVGKEFGVATNTFFKPPCVSVYRCGGCCISEGLQCMNTSTSYLSKTLFEITVPLSQGPKPVTISFANHTSCRCMSKL

SEQ ID NO: 63 - protein, 101 aa, synthetic construct
NTEILKSIDNEWRKTQCMPREVAIDVGKEFGVATNTFFKPPCVSVYRCGGCCIGEGLQCMNTSTSYLSKTLFEITVPLSQGPKPVTISFANHTSCRCMSKL

SEQ ID NO: 64 - protein, 101 aa, synthetic construct
NTEILKSIDNEWRKTQCMPREVAIDVGKEFGVATNTFFKPPCVSVYRCGGCCISEGLQCMNTSTSYLSKTLFEITVPISQGPKPVTISFANHTSCRCMSKL

SEQ ID NO: 65 - protein, 101 aa, synthetic construct
NTEILKSIDNEWRKTQCMPREVAIDVGKEFGVATNTFFKPPCVSVYRCGGCCIGEGLQCMNTSTSYLSKTLFEITVPISQGPKPVTISFANHTSCRCMSKL

SEQ ID NO: 66 - protein, 101 aa, synthetic construct
NTEILKSIDNEWRKTQCMPREVAIDVGKEFGVATNTFFKPPCVSVYRCGGCCISEGLQCMNTSTSYLSKTLFEITTPISQGPKPVTISFANHTSCRCMSKL

SEQ ID NO: 67 - protein, 101 aa, synthetic construct
NTEILKSIDNEWRKTQCMPREVAIDVGKEFGVATNTFFKPPCVSVYRCGGCCIGEGLQCMNTSTSYLSKTLFEITTPISQGPKPVTISFANHTSCRCMSKL

SEQ ID NO: 68 - protein, 101 aa, synthetic construct
NTEILKSIDNEWRKTQCMPREVAIDVGKEFGVATNTFFKPPCVSVYRCGGCCNGEGLQCMNTSTSYLSKTLFEITVPLSQGPKPVTISFANHTSCRCMSKL

SEQ ID NO: 69 - protein, 101 aa, synthetic construct
NTEILKSIDNEWRKTQCMPREVAIDVGKEFGVATNTFFKPPCVSVYRCGGCCNSEGLQCMNTSTSYLSKTLFEITVPISQGPKPVTISFANHTSCRCMSKL

SEQ ID NO: 70 - protein, 101 aa, synthetic construct
NTEILKSIDNEWRKTQCMPREVAIDVGKEFGVATNTFFKPPCVSVYRCGGCCNGEGLQCMNTSTSYLSKTLFEITVPISQGPKPVTISFANHTSCRCMSKL

SEQ ID NO: 71 - protein, 101 aa, synthetic construct
NTEILKSIDNEWRKTQCMPREVAIDVGKEFGVATNTFFKPPCVSVYRCGGCCNSEGLQCMNTSTSYLSKTLFEITTPISQGPKPVTISFANHTSCRCMSKL

SEQ ID NO: 72 - protein, 101 aa, synthetic construct
NTEILKSIDNEWRKTQCMPREVAIDVGKEFGVATNTFFKPPCVSVYRCGGCCNGEGLQCMNTSTSYLSKTLFEITTPISQGPKPVTISFANHTSCRCMSKL

SEQ ID NO: 73 - protein, 101 aa, synthetic construct
NTEIEKSINNEWRKTKCMPREVAIDVGKEFGVATNTFFKPPCVSVYRCGGCCNSEGLQCMNTSTSYLSKTLFEITVPLSQGPKPVTISFANHTSCRCMSKL

SEQ ID NO: 74 - protein, 101 aa, synthetic construct
NTEIMKSIDNEWRKTQCMPREVAIDVGKEFGVATNTFFKPPCVSVYRCGGCCRSEGLQCMNTSTSYLSKTLFEITVPLSQGPKPVTISFANHTSCRCMSKL

SEQ ID NO: 75 - protein, 101 aa, synthetic construct
NTEIEKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKL

SEQ ID NO: 76 - protein, 101 aa, synthetic construct
NTEIEKSIDNEWRKTQCMPREVAIDVGKEFGVATNTFFKPPCVSVYRCGGCCRSEGLQCMNTSTSYLSKTLFEITVPLSQGPKPVTISFANHTSCRCMSKL

SEQ ID NO: 77 - protein, 101 aa, synthetic construct
NTEIEKSIDNEWRKTQCMPREVAIDVGKEFGVATNTFFKPPCVSVYRCGGCCRGEGLQCMNTSTSYLSKTLFEITVPLSQGPKPVTISFANHTSCRCMSKL

SEQ ID NO: 78 - protein, 101 aa, synthetic construct
NTEIEKSIDNEWRKTQCMPREVAIDVGKEFGVATNTFFKPPCVSVYRCGGCCNGEGLQCMNTSTSYLSKTLFEITVPLSQGPKPVTISFANHTSCRCMSKL

SEQ ID NO: 79 - protein, 101 aa, synthetic construct
NTEILKSIDNEWRKTQCMPREVAIDVGKEFGVATNTFFKPPCVSVYRCGGCCHSEGLQCMNTSTSYLSKTLFEITTPISQGPKPVTISFANHTSCRCMSKL

SEQ ID NO: 80 - protein, 101 aa, synthetic construct
NTEILKSIDNEWRKTQCMPREVAIDVGKEFGVATNTFFKPPCVSVYRCGGCCHSEGLQCMNTSTSYLSKTLFEITVPLSQGPKPVTISFANHTSCRCMSKL

SEQ ID NO: 81 - protein, 101 aa, synthetic construct
NTEILKSIDNEWRKTQCMPREVAIDVGKEFGVATNTFFKPPCVSVYRCGGCCQREGLQCMNTSTSYLSKTLFEITTPISQGPKPVTISFANHTSCRCMSKL

SEQ ID NO: 82 - protein, 101 aa, synthetic construct
NTEILKSIDNEWRKTQCMPREVAIDVGKEFGVATNTFFKPPCVSVYRCGGCCNREGLQCMNTSTSYLSKTLFEITVPLSQGPKPVTISFANHTSCRCMSKL

SEQ ID NO: 83 - protein, 101 aa, synthetic construct
NTEILKSIDNEWRKTQCMPREVAIDVGKEFGVATNTFFKPPCVSVYRCGGCCRREGLQCMNTSTSYLSKTLFEITVPLSQGPKPVTISFANHTSCRCMSKL

SEQ ID NO: 84 - protein, 101 aa, synthetic construct
NTEILKSIDNEWRKTQCMPREVAIDVGKEFGVATNTFFKPPCVSVYRCGGCCRREGLQCMNTSTSYLSKTLFEITVPISQGPKPVTISFANHTSCRCMSKL

SEQ ID NO: 85 - protein, 101 aa, synthetic construct
NTEILKSIDNEWRKTQCMPREVAIDVGKEFGVATNTFFKPPCVSVYRCGGCCRREGLQCMNTSTSYLSKTLFEITTPISQGPKPVTISFANHTSCRCMSKL

SEQ ID NO: 86 - protein, 101 aa, synthetic construct
NTEILKSIDNEWRKTQCMPREVAIDVGKEFGVATNTFFKPPCVSVYRCGGCCQREGLQCMNTSTSYLSKTLFEITVPLSQGPKPVTISFANHTSCRCMSKL

SEQ ID NO: 87 - protein, 101 aa, synthetic construct
NTEILKSIDNEWRKTQCMPREVAIDVGKEFGVATNTFFKPPCVSVYRCGGCCQREGLQCMNTSTSYLSKTLFEITVPISQGPKPVTISFANHTSCRCMSKL

SEQ ID NO: 88 - protein, 101 aa, synthetic construct
NTEILKSIDNEWRKTQCMPREVAIDVGKEFGVATNTFFKPPCVSVYRCGGCCIREGLQCMNTSTSYLSKTLFEITVPLSQGPKPVTISFANHTSCRCMSKL

SEQ ID NO: 89 - protein, 101 aa, synthetic construct
NTEILKSIDNEWRKTQCMPREVAIDVGKEFGVATNTFFKPPCVSVYRCGGCCIREGLQCMNTSTSYLSKTLFEITVPISQGPKPVTISFANHTSCRCMSKL

SEQ ID NO: 90 - protein, 101 aa, synthetic construct
NTEILKSIDNEWRKTQCMPREVAIDVGKEFGVATNTFFKPPCVSVYRCGGCCIREGLQCMNTSTSYLSKTLFEITTPISQGPKPVTISFANHTSCRCMSKL

SEQ ID NO: 91 - protein, 101 aa, synthetic construct
NTEILKSIDNEWRKTQCMPREVAIDVGKEFGVATNTFFKPPCVSVYRCGGCCHREGLQCMNTSTSYLSKTLFEITVPLSQGPKPVTISFANHTSCRCMSKL

SEQ ID NO: 92 - protein, 101 aa, synthetic construct
NTEILKSIDNEWRKTQCMPREVAIDVGKEFGVATNTFFKPPCVSVYRCGGCCNREGLQCMNTSTSYLSKTLFEITVPISQGPKPVTISFANHTSCRCMSKL

SEQ ID NO: 93 - protein, 101 aa, synthetic construct
NTEIEKSIDNEWRKTQCMPREVAIDVGKEFGVATNTFFKPPCVSVYRCGGCCRREGLQCMNTSTSYLSKTLFEITVPLSQGPKPVTISFANHTSCRCMSKL

SEQ ID NO: 94 - protein, 101 aa, synthetic construct
NTEIEKSIDNEWRKTQCMPREVAIDVGKEFGVATNTFFKPPCVSVYRCGGCCQSEGLQCMNTSTSYLSKTLFEITVPLSQGPKPVTISFANHTSCRCMSKL

SEQ ID NO: 95 - protein, 101 aa, synthetic construct
NTEIEKSIDNEWRKTQCMPREVAIDVGKEFGVATNTFFKPPCVSVYRCGGCCISEGLQCMNTSTSYLSKTLFEITVPLSQGPKPVTISFANHTSCRCMSKL

SEQ ID NO: 96 - protein, 101 aa, synthetic construct
NTEIEKSIDNEWRKTQCMPREVAIDVGKEFGVATNTFFKPPCVSVYRCGGCCQREGLQCMNTSTSYLSKTLFEITVPLSQGPKPVTISFANHTSCRCMSKL

SEQ ID NO: 97 - protein, 101 aa, synthetic construct
NTEIEKSIDNEWRKTQCMPREVAIDVGKEFGVATNTFFKPPCVSVYRCGGCCIREGLQCMNTSTSYLSKTLFEITVPLSQGPKPVTISFANHTSCRCMSKL

SEQ ID NO: 98 - protein, 101 aa, synthetic construct
NTEIEKSIDNEWRKTQCMPREVAIDVGKEFGVATNTFFKPPCVSVYRCGGCCHREGLQCMNTSTSYLSKTLFEITVPLSQGPKPVTISFANHTSCRCMSKL

SEQ ID NO: 99 - protein, 101 aa, synthetic construct
NTEIEKSIDNEWRKTQCMPREVAIDVGKEFGVATNTFFKPPCVSVYRCGGCCRREGLQCMNTSTSYLSKTLFEITTPISQGPKPVTISFANHTSCRCMSKL

SEQ ID NO: 100 - protein, 101 aa, synthetic construct
NTEILKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKL

SEQ ID NO: 101 - protein, 101 aa, synthetic construct
NTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKL

SEQ ID NO: 102 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcagatctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 103 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcagaggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 104 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcagatctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 105 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcagaggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 106 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcagatctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 107 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcagaggcgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 108 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 109 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 110 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 111 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 112 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 113 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagggcgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 114 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 115 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 116 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 117 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 118 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 119 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcggcgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 120 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 121 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 122 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 123 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 124 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 125 - DNA, 303 bp, synthetic construct
  source 1..303: mol_type = other DNA
aacacggaaatcgagaagtctataaacaatgagtggcgaaagacaaagtgtatgccgcgcgaggtagcaatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 126 - DNA, 303 bp, synthetic construct
  source 1..303: mol_type = other DNA
aacacggaaatcatgaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcagatctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 127 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 128 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaggtctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 129 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaggggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 130 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 131 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccactctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 132 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccactctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 133 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagagagaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 134 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaatagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 135 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 136 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 137 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacaacaccaatctcccaaggaccgaagcctgtgactatcagctttgctaaccatacaagctgtcgctgtatgagcaaactg

SEQ ID NO: 138 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 139 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagagagaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 140 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 141 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 142 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 143 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccacagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 144 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaatagagaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 145 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 146 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 147 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 148 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 149 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 150 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccacagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 151 - DNA, 303 bp, synthetic construct
  source 1..303: mol_type = other DNA
aacacggaaatcgagaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacaacaccaatctcccaaggaccgaagcctgtgactatcagctttgctaaccatacaagctgtcgctgtatgagcaaactg

SEQ ID NO: 152 - DNA, 303 bp, synthetic construct
  source 1..303: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 153 - DNA, 303 bp, Homo sapiens
  source 1..303: mol_type = genomic DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 154 - protein, 101 aa, synthetic construct
NTEILKSIDNEWRKTQCMPREVCIDVGKEFGVATNTFFKPPCVSVYRCGGCCRSEGLQCMNTSTSYLSKTLFEITVPLSQGPKPVTISFANHTSCRCMSKL

SEQ ID NO: 155 - protein, 101 aa, synthetic construct
NTEILKSIDNEWRKTQCMPREVCIDVGKEFGVATNTFFKPPCVSVYRCGGCCRGEGLQCMNTSTSYLSKTLFEITVPLSQGPKPVTISFANHTSCRCMSKL

SEQ ID NO: 156 - protein, 101 aa, synthetic construct
NTEILKSIDNEWRKTQCMPREVCIDVGKEFGVATNTFFKPPCVSVYRCGGCCRSEGLQCMNTSTSYLSKTLFEITVPISQGPKPVTISFANHTSCRCMSKL

SEQ ID NO: 157 - protein, 101 aa, synthetic construct
NTEILKSIDNEWRKTQCMPREVCIDVGKEFGVATNTFFKPPCVSVYRCGGCCRGEGLQCMNTSTSYLSKTLFEITVPISQGPKPVTISFANHTSCRCMSKL

SEQ ID NO: 158 - protein, 101 aa, synthetic construct
NTEILKSIDNEWRKTQCMPREVCIDVGKEFGVATNTFFKPPCVSVYRCGGCCRSEGLQCMNTSTSYLSKTLFEITTPISQGPKPVTISFANHTSCRCMSKL

SEQ ID NO: 159 - protein, 101 aa, synthetic construct
NTEILKSIDNEWRKTQCMPREVCIDVGKEFGVATNTFFKPPCVSVYRCGGCCRGEGLQCMNTSTSYLSKTLFEITTPISQGPKPVTISFANHTSCRCMSKL

SEQ ID NO: 160 - protein, 101 aa, synthetic construct
NTEILKSIDNEWRKTQCMPREVCIDVGKEFGVATNTFFKPPCVSVYRCGGCCQSEGLQCMNTSTSYLSKTLFEITVPLSQGPKPVTISFANHTSCRCMSKL

SEQ ID NO: 161 - protein, 101 aa, synthetic construct
NTEILKSIDNEWRKTQCMPREVCIDVGKEFGVATNTFFKPPCVSVYRCGGCCQGEGLQCMNTSTSYLSKTLFEITVPLSQGPKPVTISFANHTSCRCMSKL

SEQ ID NO: 162 - protein, 101 aa, synthetic construct
NTEILKSIDNEWRKTQCMPREVCIDVGKEFGVATNTFFKPPCVSVYRCGGCCQSEGLQCMNTSTSYLSKTLFEITVPISQGPKPVTISFANHTSCRCMSKL

SEQ ID NO: 163 - protein, 101 aa, synthetic construct
NTEILKSIDNEWRKTQCMPREVCIDVGKEFGVATNTFFKPPCVSVYRCGGCCQGEGLQCMNTSTSYLSKTLFEITVPISQGPKPVTISFANHTSCRCMSKL

SEQ ID NO: 164 - protein, 101 aa, synthetic construct
NTEILKSIDNEWRKTQCMPREVCIDVGKEFGVATNTFFKPPCVSVYRCGGCCQSEGLQCMNTSTSYLSKTLFEITTPISQGPKPVTISFANHTSCRCMSKL

SEQ ID NO: 165 - protein, 101 aa, synthetic construct
NTEILKSIDNEWRKTQCMPREVCIDVGKEFGVATNTFFKPPCVSVYRCGGCCQGEGLQCMNTSTSYLSKTLFEITTPISQGPKPVTISFANHTSCRCMSKL

SEQ ID NO: 166 - protein, 101 aa, synthetic construct
NTEILKSIDNEWRKTQCMPREVCIDVGKEFGVATNTFFKPPCVSVYRCGGCCISEGLQCMNTSTSYLSKTLFEITVPLSQGPKPVTISFANHTSCRCMSKL

SEQ ID NO: 167 - protein, 101 aa, synthetic construct
NTEILKSIDNEWRKTQCMPREVCIDVGKEFGVATNTFFKPPCVSVYRCGGCCIGEGLQCMNTSTSYLSKTLFEITVPLSQGPKPVTISFANHTSCRCMSKL

SEQ ID NO: 168 - protein, 101 aa, synthetic construct
NTEILKSIDNEWRKTQCMPREVCIDVGKEFGVATNTFFKPPCVSVYRCGGCCISEGLQCMNTSTSYLSKTLFEITVPISQGPKPVTISFANHTSCRCMSKL

SEQ ID NO: 169 - protein, 101 aa, synthetic construct
NTEILKSIDNEWRKTQCMPREVCIDVGKEFGVATNTFFKPPCVSVYRCGGCCIGEGLQCMNTSTSYLSKTLFEITVPISQGPKPVTISFANHTSCRCMSKL

SEQ ID NO: 170 - protein, 101 aa, synthetic construct
NTEILKSIDNEWRKTQCMPREVCIDVGKEFGVATNTFFKPPCVSVYRCGGCCISEGLQCMNTSTSYLSKTLFEITTPISQGPKPVTISFANHTSCRCMSKL

SEQ ID NO: 171 - protein, 101 aa, synthetic construct
NTEILKSIDNEWRKTQCMPREVCIDVGKEFGVATNTFFKPPCVSVYRCGGCCIGEGLQCMNTSTSYLSKTLFEITTPISQGPKPVTISFANHTSCRCMSKL

SEQ ID NO: 172 - protein, 101 aa, synthetic construct
NTEILKSIDNEWRKTQCMPREVCIDVGKEFGVATNTFFKPPCVSVYRCGGCCNGEGLQCMNTSTSYLSKTLFEITVPLSQGPKPVTISFANHTSCRCMSKL

SEQ ID NO: 173 - protein, 101 aa, synthetic construct
NTEILKSIDNEWRKTQCMPREVCIDVGKEFGVATNTFFKPPCVSVYRCGGCCNSEGLQCMNTSTSYLSKTLFEITVPISQGPKPVTISFANHTSCRCMSKL

SEQ ID NO: 174 - protein, 101 aa, synthetic construct
NTEILKSIDNEWRKTQCMPREVCIDVGKEFGVATNTFFKPPCVSVYRCGGCCNGEGLQCMNTSTSYLSKTLFEITVPISQGPKPVTISFANHTSCRCMSKL

SEQ ID NO: 175 - protein, 101 aa, synthetic construct
NTEILKSIDNEWRKTQCMPREVCIDVGKEFGVATNTFFKPPCVSVYRCGGCCNSEGLQCMNTSTSYLSKTLFEITTPISQGPKPVTISFANHTSCRCMSKL

SEQ ID NO: 176 - protein, 101 aa, synthetic construct
NTEILKSIDNEWRKTQCMPREVCIDVGKEFGVATNTFFKPPCVSVYRCGGCCNGEGLQCMNTSTSYLSKTLFEITTPISQGPKPVTISFANHTSCRCMSKL

SEQ ID NO: 177 - protein, 101 aa, synthetic construct
NTEIEKSINNEWRKTKCMPREVCIDVGKEFGVATNTFFKPPCVSVYRCGGCCNSEGLQCMNTSTSYLSKTLFEITVPLSQGPKPVTISFANHTSCRCMSKL

SEQ ID NO: 178 - protein, 101 aa, synthetic construct
NTEIMKSIDNEWRKTQCMPREVCIDVGKEFGVATNTFFKPPCVSVYRCGGCCRSEGLQCMNTSTSYLSKTLFEITVPLSQGPKPVTISFANHTSCRCMSKL

SEQ ID NO: 179 - protein, 101 aa, synthetic construct
NTEIEKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKL

SEQ ID NO: 180 - protein, 101 aa, synthetic construct
NTEIEKSIDNEWRKTQCMPREVCIDVGKEFGVATNTFFKPPCVSVYRCGGCCRSEGLQCMNTSTSYLSKTLFEITVPLSQGPKPVTISFANHTSCRCMSKL

SEQ ID NO: 181 - protein, 101 aa, synthetic construct
NTEIEKSIDNEWRKTQCMPREVCIDVGKEFGVATNTFFKPPCVSVYRCGGCCRGEGLQCMNTSTSYLSKTLFEITVPLSQGPKPVTISFANHTSCRCMSKL

SEQ ID NO: 182 - protein, 101 aa, synthetic construct
NTEIEKSIDNEWRKTQCMPREVCIDVGKEFGVATNTFFKPPCVSVYRCGGCCNGEGLQCMNTSTSYLSKTLFEITVPLSQGPKPVTISFANHTSCRCMSKL

SEQ ID NO: 183 - protein, 101 aa, synthetic construct
NTEILKSIDNEWRKTQCMPREVCIDVGKEFGVATNTFFKPPCVSVYRCGGCCHSEGLQCMNTSTSYLSKTLFEITTPISQGPKPVTISFANHTSCRCMSKL

SEQ ID NO: 184 - protein, 101 aa, synthetic construct
NTEILKSIDNEWRKTQCMPREVCIDVGKEFGVATNTFFKPPCVSVYRCGGCCHSEGLQCMNTSTSYLSKTLFEITVPLSQGPKPVTISFANHTSCRCMSKL

SEQ ID NO: 185 - protein, 101 aa, synthetic construct
NTEILKSIDNEWRKTQCMPREVCIDVGKEFGVATNTFFKPPCVSVYRCGGCCQREGLQCMNTSTSYLSKTLFEITTPISQGPKPVTISFANHTSCRCMSKL

SEQ ID NO: 186 - protein, 101 aa, synthetic construct
NTEILKSIDNEWRKTQCMPREVCIDVGKEFGVATNTFFKPPCVSVYRCGGCCNREGLQCMNTSTSYLSKTLFEITVPLSQGPKPVTISFANHTSCRCMSKL

SEQ ID NO: 187 - protein, 101 aa, synthetic construct
NTEILKSIDNEWRKTQCMPREVCIDVGKEFGVATNTFFKPPCVSVYRCGGCCRREGLQCMNTSTSYLSKTLFEITVPLSQGPKPVTISFANHTSCRCMSKL

SEQ ID NO: 188 - protein, 101 aa, synthetic construct
NTEILKSIDNEWRKTQCMPREVCIDVGKEFGVATNTFFKPPCVSVYRCGGCCRREGLQCMNTSTSYLSKTLFEITVPISQGPKPVTISFANHTSCRCMSKL

SEQ ID NO: 189 - protein, 101 aa, synthetic construct
NTEILKSIDNEWRKTQCMPREVCIDVGKEFGVATNTFFKPPCVSVYRCGGCCRREGLQCMNTSTSYLSKTLFEITTPISQGPKPVTISFANHTSCRCMSKL

SEQ ID NO: 190 - protein, 101 aa, synthetic construct
NTEILKSIDNEWRKTQCMPREVCIDVGKEFGVATNTFFKPPCVSVYRCGGCCQREGLQCMNTSTSYLSKTLFEITVPLSQGPKPVTISFANHTSCRCMSKL

SEQ ID NO: 191 - protein, 101 aa, synthetic construct
NTEILKSIDNEWRKTQCMPREVCIDVGKEFGVATNTFFKPPCVSVYRCGGCCQREGLQCMNTSTSYLSKTLFEITVPISQGPKPVTISFANHTSCRCMSKL

SEQ ID NO: 192 - protein, 101 aa, synthetic construct
NTEILKSIDNEWRKTQCMPREVCIDVGKEFGVATNTFFKPPCVSVYRCGGCCIREGLQCMNTSTSYLSKTLFEITVPLSQGPKPVTISFANHTSCRCMSKL

SEQ ID NO: 193 - protein, 101 aa, synthetic construct
NTEILKSIDNEWRKTQCMPREVCIDVGKEFGVATNTFFKPPCVSVYRCGGCCIREGLQCMNTSTSYLSKTLFEITVPISQGPKPVTISFANHTSCRCMSKL

SEQ ID NO: 194 - protein, 101 aa, synthetic construct
NTEILKSIDNEWRKTQCMPREVCIDVGKEFGVATNTFFKPPCVSVYRCGGCCIREGLQCMNTSTSYLSKTLFEITTPISQGPKPVTISFANHTSCRCMSKL

SEQ ID NO: 195 - protein, 101 aa, synthetic construct
NTEILKSIDNEWRKTQCMPREVCIDVGKEFGVATNTFFKPPCVSVYRCGGCCHREGLQCMNTSTSYLSKTLFEITVPLSQGPKPVTISFANHTSCRCMSKL

SEQ ID NO: 196 - protein, 101 aa, synthetic construct
NTEILKSIDNEWRKTQCMPREVCIDVGKEFGVATNTFFKPPCVSVYRCGGCCNREGLQCMNTSTSYLSKTLFEITVPISQGPKPVTISFANHTSCRCMSKL

SEQ ID NO: 197 - protein, 101 aa, synthetic construct
NTEIEKSIDNEWRKTQCMPREVCIDVGKEFGVATNTFFKPPCVSVYRCGGCCRREGLQCMNTSTSYLSKTLFEITVPLSQGPKPVTISFANHTSCRCMSKL

SEQ ID NO: 198 - protein, 101 aa, synthetic construct
NTEIEKSIDNEWRKTQCMPREVCIDVGKEFGVATNTFFKPPCVSVYRCGGCCQSEGLQCMNTSTSYLSKTLFEITVPLSQGPKPVTISFANHTSCRCMSKL

SEQ ID NO: 199 - protein, 101 aa, synthetic construct
NTEIEKSIDNEWRKTQCMPREVCIDVGKEFGVATNTFFKPPCVSVYRCGGCCISEGLQCMNTSTSYLSKTLFEITVPLSQGPKPVTISFANHTSCRCMSKL

SEQ ID NO: 200 - protein, 101 aa, synthetic construct
NTEIEKSIDNEWRKTQCMPREVCIDVGKEFGVATNTFFKPPCVSVYRCGGCCQREGLQCMNTSTSYLSKTLFEITVPLSQGPKPVTISFANHTSCRCMSKL

SEQ ID NO: 201 - protein, 101 aa, synthetic construct
NTEIEKSIDNEWRKTQCMPREVCIDVGKEFGVATNTFFKPPCVSVYRCGGCCIREGLQCMNTSTSYLSKTLFEITVPLSQGPKPVTISFANHTSCRCMSKL

SEQ ID NO: 202 - protein, 101 aa, synthetic construct
NTEIEKSIDNEWRKTQCMPREVCIDVGKEFGVATNTFFKPPCVSVYRCGGCCHREGLQCMNTSTSYLSKTLFEITVPLSQGPKPVTISFANHTSCRCMSKL

SEQ ID NO: 203 - protein, 101 aa, synthetic construct
NTEIEKSIDNEWRKTQCMPREVCIDVGKEFGVATNTFFKPPCVSVYRCGGCCRREGLQCMNTSTSYLSKTLFEITTPISQGPKPVTISFANHTSCRCMSKL

SEQ ID NO: 204 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcagatctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 205 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcagaggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 206 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcagatctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 207 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcagaggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 208 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcagatctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 209 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcagaggcgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 210 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 211 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 212 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 213 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 214 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 215 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagggcgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 216 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 217 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatcggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 218 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 219 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatcggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 220 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 221 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatcggcgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 222 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 223 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 224 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 225 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 226 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 227 - DNA, 303 bp, synthetic construct
  source 1..303: mol_type = other DNA
aacacggaaatcgagaagtctataaacaatgagtggcgaaagacaaagtgtatgccgcgcgaggtatgtatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 228 - DNA, 303 bp, synthetic construct
  source 1..303: mol_type = other DNA
aacacggaaatcatgaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcagatctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 229 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 230 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaggtctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 231 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaggggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 232 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 233 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccactctgaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 234 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccactctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 235 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagagagaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 236 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaatagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 237 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 238 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 239 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacaacaccaatctcccaaggaccgaagcctgtgactatcagctttgctaaccatacaagctgtcgctgtatgagcaaactg

SEQ ID NO: 240 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 241 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagagagaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 242 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 243 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 244 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacaaccccaatctcccaaggaccgaagcctgtgactatcagctttgctaaccatacaagctgtcgctgtatgagcaaactg

SEQ ID NO: 245 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccacagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 246 - DNA, 303 bp, synthetic construct
  source 1..303: mol_type = other DNA
aacacggaaatcctcaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcaatagagaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactg

SEQ ID NO: 247 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 248 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 249 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 250 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccagagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 251 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 252 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccacagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactg

SEQ ID NO: 253 - DNA, 303 bp, synthetic construct
  source 1..303: mol_type = other DNA
aacacggaaatcgagaagtctatagataatgagtggcgaaagacacaatgtatgccgcgcgaggtatgtatcgacgttggtaaagaatttggtgtagcaacgaacacattcttcaaacccccttgtgtgagtgtatatagatgtggagggtgttgccggagagaggggctccaatgtatgaacacgagtacgtcttacttgagtaagaccttgttcgaaattacaacaccaatctcccaaggaccgaagcctgtgactatcagctttgctaaccatacaagctgtcgctgtatgagcaaactg

SEQ ID NO: 254 - protein, 109 aa, synthetic construct
NTEILKSIDNEWRKTQCMPREVAIDVGKEFGAATNTFFKPPCVSVYRCGGCCISEGLQCMNTSTGYLSKTLFEITVPLSQGPKPVTISFANHTSCRCMSKLHHHHHHHH

SEQ ID NO: 255 - protein, 109 aa, synthetic construct
NTEILKSIDNEWRKTQCMPREVAIDVGKEFGAATNTFFKPPCVSVYRCGGCCRSEGLQCMNTSTGYLSKTLFEITTPISQGPKPVTISFANHTSCRCMSKLHHHHHHHH

SEQ ID NO: 256 - protein, 342 aa, synthetic construct
NTEILKSIDNEWRKTQCMPREVAIDVGKEFGAATNTFFKPPCVSVYRCGGCCISEGLQCMNTSTGYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 257 - protein, 342 aa, synthetic construct
NTEILKSIDNEWRKTQCMPREVAIDVGKEFGAATNTFFKPPCVSVYRCGGCCRSEGLQCMNTSTGYLSKTLFEITTPISQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 258 - protein, 715 aa, synthetic construct
NTEILKSIDNEWRKTQCMPREVAIDVGKEFGAATNTFFKPPCVSVYRCGGCCISEGLQCMNTSTGYLSKTLFEITVPLSQGPKPVTISFANHTSCRCMSKLAAAGGGGGSGGGGSGGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGSHHHHHHHH

SEQ ID NO: 259 - protein, 715 aa, synthetic construct
NTEILKSIDNEWRKTQCMPREVAIDVGKEFGAATNTFFKPPCVSVYRCGGCCRSEGLQCMNTSTGYLSKTLFEITTPISQGPKPVTISFANHTSCRCMSKLAAAGGGGGSGGGGSGGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGSHHHHHHHH

SEQ ID NO: 260 - DNA, 327 bp, synthetic construct
  source 1..327: mol_type = other DNA
aacaccgaaatcttgaaatcaatagataacgaatggcgcaaaactcaatgtatgccacgggaggttgcaatagacgtgggaaaggaatttggcgccgccacgaatacctttttcaagcctccctgtgtgagtgtttacagatgtggtggttgttgcatatcagagggattgcagtgcatgaacacaagtacaggttacttgagtaaaacattgtttgaaatcacagtaccattgtcccaaggccctaagccggttacgatctctttcgccaatcatacgtcatgccgctgtatgagtaagttgcatcaccatcaccatcatcaccat

SEQ ID NO: 261 - DNA, 327 bp, synthetic construct
  source 1..327: mol_type = other DNA
aacaccgaaatcttgaaatcaatagataacgaatggcgaaaaacccaatgtatgccacgcgaggtagcgatagatgtgggcaaagaattcggcgccgcgacgaacacctttttcaagcccccttgcgtctccgtatatagatgcggtggatgttgccgatccgagggccttcagtgtatgaacacatctactggctatttgagcaagacgctctttgagattacaacaccaattagtcaaggtcccaagcctgttaccatctctttcgctaaccacacttcatgccgctgtatgagtaagttgcatcaccatcaccatcatcaccat

SEQ ID NO: 262 - DNA, 1026 bp, synthetic construct
  source 1..1026: mol_type = other DNA
aacaccgaaatcttgaaatcaatagataacgaatggcgcaaaactcaatgtatgccacgggaggttgcaatagacgtgggaaaggaatttggcgccgccacgaatacctttttcaagcctccctgtgtgagtgtttacagatgtggtggttgttgcatatcagagggattgcagtgcatgaacacaagtacaggttacttgagtaaaacattgtttgaaatcacagtaccattgtcccaaggccctaagccggttacgatctctttcgccaatcatacgtcatgccgctgtatgagtaagttggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 263 - DNA, 1026 bp, synthetic construct
  source 1..1026: mol_type = other DNA
aacaccgaaatcttgaaatcaatagataacgaatggcgaaaaacccaatgtatgccacgcgaggtagcgatagatgtgggcaaagaattcggcgccgcgacgaacacctttttcaagcccccttgcgtctccgtatatagatgcggtggatgttgccgatccgagggccttcagtgtatgaacacatctactggctatttgagcaagacgctctttgagattacaacaccaattagtcaaggtcccaagcctgttaccatctctttcgctaaccacacttcatgccgctgtatgagtaagttggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 264 - DNA, 2145 bp, synthetic construct
  source 1..2145: mol_type = other DNA
aacaccgaaatcttgaaatcaatagataacgaatggcgcaaaactcaatgtatgccacgggaggttgcaatagacgtgggaaaggaatttggcgccgccacgaatacctttttcaagcctccctgtgtgagtgtttacagatgtggtggttgttgcatatcagagggattgcagtgcatgaacacaagtacaggttacttgagtaaaacattgtttgaaatcacagtaccattgtcccaaggccctaagccggttacgatctctttcgccaatcatacgtcatgccgctgtatgagtaagttggcggccgctggtggcggaggtggctctggtggaggcggtagcggcggtgggggatccgaagctcacaaaagtgagatagcacatcgctacaacgaccttggtgaacaacattttaaaggcctggtccttatcgcattttcccagtacctccagaaatgcagttatgatgagcacgctaaattggtccaggaagttacggactttgccaagacctgcgtggctgatgagtccgctgccaattgcgacaaatcccttcacacacttttcggcgacaagctctgtgcaattcctaacttgcgcgagaactacggtgagctcgcagattgctgcacaaagcaggaacctgagcgcaatgaatgcttcttgcaacataaggatgataacccgagccttccgccattcgaaagacccgaagccgaagcaatgtgtacgagtttcaaggaaaaccctacgacttttatgggacattatctgcatgaggtcgctcggagacatccttatttttacgctcccgaattgctttattatgcggaacagtataatgagatattgacccagtgctgcgccgaagctgataaggaatcctgcctcacacccaaattggatggggtgaaggagaaagcacttgtatcatcagtgcgacaaagaatgaaatgttccagcatgcaaaagtttggcgaacgcgcattcaaggcttgggctgtggcccgcctttctcagacatttcccaacgctgattttgccgagatcacgaagttggcgactgacctgacaaaggtgaataaggagtgttgtcacggtgatttgttggagtgcgctgatgatcgggccgagcttgcgaagtacatgtgtgagaatcaagcgacgataagttccaaactccagacatgctgtgataagccgcttttgaaaaaagcacattgtctgagtgaggtagaacacgataccatgccagccgatctgccggcgatagcagccgatttcgtcgaggaccaggaagtttgcaagaattatgcggaagcgaaggatgtgtttctggggacttttctgtatgaatacagtcgaagacacccggactatagtgtatccttgcttttgagacttgccaagaagtacgaagctacgctggagaaatgttgtgcagaggctaacccacccgcctgttatggtaccgtgctcgctgaattccaacccctggtcgaagaacctaagaatttggtgaaaaccaattgtgacctttatgagaaactcggcgagtatgggttccagaacgccattcttgtccgctatacccagaaagcaccacaagttagcacaccgaccctggtagaagccgcgagaaatctgggtcgagttggtactaaatgttgcaccctgcctgaagatcagaggttgccgtgtgtcgaagactacctgagtgctattttgaaccgggtgtgcctcctgcacgaaaaaactcccgtaagtgagcatgttacgaaatgctgctcaggaagcctggtagaacgcagaccatgtttttcagccctgaccgtagatgaaacctatgttcctaaagagtttaaagcagaaacttttacctttcactccgatatatgcacgctcccagaaaaggagaagcaaattaaaaaacaaactgcactggcggagttggtcaaacacaaacccaaggcgactgccgagcaactcaaaacagtgatggatgatttcgcccagttcctcgacacctgctgtaaagcggcggacaaggatacttgtttctccacagagggaccaaatctggttacgagatgtaaggatgccttggctggcggcagtcatcaccatcaccatcatcaccat

SEQ ID NO: 265 - DNA, 2145 bp, synthetic construct
  source 1..2145: mol_type = other DNA
aacaccgaaatcttgaaatcaatagataacgaatggcgaaaaacccaatgtatgccacgcgaggtagcgatagatgtgggcaaagaattcggcgccgcgacgaacacctttttcaagcccccttgcgtctccgtatatagatgcggtggatgttgccgatccgagggccttcagtgtatgaacacatctactggctatttgagcaagacgctctttgagattacaacaccaattagtcaaggtcccaagcctgttaccatctctttcgctaaccacacttcatgccgctgtatgagtaagttggcggccgctggtggcggaggtggctctggtggaggcggtagcggcggtgggggatccgaagctcacaaaagtgagatagcacatcgctacaacgaccttggtgaacaacattttaaaggcctggtccttatcgcattttcccagtacctccagaaatgcagttatgatgagcacgctaaattggtccaggaagttacggactttgccaagacctgcgtggctgatgagtccgctgccaattgcgacaaatcccttcacacacttttcggcgacaagctctgtgcaattcctaacttgcgcgagaactacggtgagctcgcagattgctgcacaaagcaggaacctgagcgcaatgaatgcttcttgcaacataaggatgataacccgagccttccgccattcgaaagacccgaagccgaagcaatgtgtacgagtttcaaggaaaaccctacgacttttatgggacattatctgcatgaggtcgctcggagacatccttatttttacgctcccgaattgctttattatgcggaacagtataatgagatattgacccagtgctgcgccgaagctgataaggaatcctgcctcacacccaaattggatggggtgaaggagaaagcacttgtatcatcagtgcgacaaagaatgaaatgttccagcatgcaaaagtttggcgaacgcgcattcaaggcttgggctgtggcccgcctttctcagacatttcccaacgctgattttgccgagatcacgaagttggcgactgacctgacaaaggtgaataaggagtgttgtcacggtgatttgttggagtgcgctgatgatcgggccgagcttgcgaagtacatgtgtgagaatcaagcgacgataagttccaaactccagacatgctgtgataagccgcttttgaaaaaagcacattgtctgagtgaggtagaacacgataccatgccagccgatctgccggcgatagcagccgatttcgtcgaggaccaggaagtttgcaagaattatgcggaagcgaaggatgtgtttctggggacttttctgtatgaatacagtcgaagacacccggactatagtgtatccttgcttttgagacttgccaagaagtacgaagctacgctggagaaatgttgtgcagaggctaacccacccgcctgttatggtaccgtgctcgctgaattccaacccctggtcgaagaacctaagaatttggtgaaaaccaattgtgacctttatgagaaactcggcgagtatgggttccagaacgccattcttgtccgctatacccagaaagcaccacaagttagcacaccgaccctggtagaagccgcgagaaatctgggtcgagttggtactaaatgttgcaccctgcctgaagatcagaggttgccgtgtgtcgaagactacctgagtgctattttgaaccgggtgtgcctcctgcacgaaaaaactcccgtaagtgagcatgttacgaaatgctgctcaggaagcctggtagaacgcagaccatgtttttcagccctgaccgtagatgaaacctatgttcctaaagagtttaaagcagaaacttttacctttcactccgatatatgcacgctcccagaaaaggagaagcaaattaaaaaacaaactgcactggcggagttggtcaaacacaaacccaaggcgactgccgagcaactcaaaacagtgatggatgatttcgcccagttcctcgacacctgctgtaaagcggcggacaaggatacttgtttctccacagagggaccaaatctggttacgagatgtaaggatgccttggctggcggcagtcatcaccatcaccatcatcaccat

SEQ ID NO: 266 - protein, 342 aa, synthetic construct
NTEILKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 267 - protein, 342 aa, synthetic construct
NTEILKSIDNEWRKTQCMPREVAIDVGKEFGVATNTFFKPPCVSVYRCGGCCQSEGLQCMNTSTSYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 268 - protein, 342 aa, synthetic construct
NTEILKSIDNEWRKTQCMPREVAIDVGKEFGVATNTFFKPPCVSVYRCGGCCQGEGLQCMNTSTSYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 269 - protein, 342 aa, synthetic construct
NTEILKSIDNEWRKTQCMPREVAIDVGKEFGVATNTFFKPPCVSVYRCGGCCQSEGLQCMNTSTSYLSKTLFEITVPISQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 270 - protein, 342 aa, synthetic construct
NTEILKSIDNEWRKTQCMPREVAIDVGKEFGVATNTFFKPPCVSVYRCGGCCQGEGLQCMNTSTSYLSKTLFEITVPISQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 271 - protein, 342 aa, synthetic construct
NTEILKSIDNEWRKTQCMPREVAIDVGKEFGVATNTFFKPPCVSVYRCGGCCISEGLQCMNTSTSYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 272 - protein, 342 aa, synthetic construct
NTEILKSIDNEWRKTQCMPREVAIDVGKEFGVATNTFFKPPCVSVYRCGGCCIGEGLQCMNTSTSYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 273 - protein, 342 aa, synthetic construct
NTEILKSIDNEWRKTQCMPREVAIDVGKEFGVATNTFFKPPCVSVYRCGGCCISEGLQCMNTSTSYLSKTLFEITVPISQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 274 - protein, 342 aa, synthetic construct
NTEILKSIDNEWRKTQCMPREVAIDVGKEFGVATNTFFKPPCVSVYRCGGCCIGEGLQCMNTSTSYLSKTLFEITVPISQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 275 - protein, 342 aa, synthetic construct
NTEILKSIDNEWRKTQCMPREVAIDVGKEFGVATNTFFKPPCVSVYRCGGCCNGEGLQCMNTSTSYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 276 - protein, 342 aa, synthetic construct
NTEILKSIDNEWRKTQCMPREVAIDVGKEFGVATNTFFKPPCVSVYRCGGCCNGEGLQCMNTSTSYLSKTLFEITVPISQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 277 - protein, 342 aa, synthetic construct
NTEILKSIDNEWRKTQCMPREVAIDVGKEFGVATNTFFKPPCVSVYRCGGCCHSEGLQCMNTSTSYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 278 - protein, 342 aa, synthetic construct
NTEILKSIDNEWRKTQCMPREVAIDVGKEFGVATNTFFKPPCVSVYRCGGCCIREGLQCMNTSTSYLSKTLFEITVPLSQGPKPVTISFANHTSCRCMSKLAAAEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 279 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaattctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 280 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 281 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 282 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagtctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 283 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccagggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 284 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 285 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 286 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatctctgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 287 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 288 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 289 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcaatggcgaggggctccaatgtatgaacacgagtacgtcttacttgagtaagaccttgttcgaaattacagtaccaatctcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 290 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgccactctgaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 291 - DNA, 1026 bp, synthetic construct
  source 1..1026: mol_type = other DNA
aacacggaaatcctcaagtctatagataatgagtggcgaaagacacaatgtatgccgcgcgaggtagcaatcgacgttggtaaagaatttggtgtagcaacgaacacattcttcaaacccccttgtgtgagtgtatatagatgtggagggtgttgcatcagagaggggctccaatgtatgaacacgagtacgtcttacttgagtaagaccttgttcgaaattacagtaccactttcccaaggaccgaagcctgtgactatcagctttgctaaccatacaagctgtcgctgtatgagcaaactggcggccgctgaacccaaaagttgtgacaaaacgcacacatgcccgccttgccccgccccggagctactgggcggaccctctgtgtttctgtttccaccaaagccgaaagatactcttatgatttccaggacacctgaagtcacctgcgttgttgtggatgttagccatgaagatcccgaagtgaagtttaactggtacgtagacggcgtggaagtacacaatgcaaaaacgaaacctagagaagaacaatatcaatcaacctatagggtagtgtcagtccttacagtcctacaccaggactggcttaatgggaaagagtataaatgcaaggtcagtaacaaggcgctacccgctccaatagagaaaactatctctaaggctaaaggccagccgcgtgagccccaggtatatacactgccaccatccagggaggaaatgacaaagaaccaggtgtccctgacttgcttagtaaaagggttttatccatccgacatcgccgtggaatgggaatctaatggtcagcctgaaaacaattacaagacaacacctcccgttcttgatagcgacggatcttttttcctttactctaaattgactgtagataaaagtaggtggcagcaaggtaacgtcttttcatgcagcgtgatgcacgaggcattacacaaccattacacacagaaatcattgtcattaagtccagggaagcaccatcaccatcaccat

SEQ ID NO: 292 - protein, 101 aa, Mus sp.; identical to SEQ ID NO: 23

SEQ ID NO: 293 - DNA, 303 bp, Mus sp.
  source 1..303: mol_type = genomic DNA
aacacagagatcctgaaaagtattgataatgagtggagaaagactcaatgcatgccacgtgaggtgtgtatagatgtggggaaggagtttggagcagccacaaacaccttctttaaacctccatgtgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactg

SEQ ID NO: 294 - protein, 16 aa, synthetic construct
GGGGGSGGGGSGGGGS

SEQ ID NO: 295 - protein, 9 aa, synthetic construct
PPGPPGPPG

SEQ ID NO: 296 - protein, 10 aa, synthetic construct
PGPGPGPGPG

SEQ ID NO: 297 - protein, 15 aa, synthetic construct
PPPPGPPPPGPPPPG

SEQ ID NO: 298 - protein, 12 aa, synthetic construct
ESESESESESES

SEQ ID NO: 299 - protein, 10 aa, synthetic construct
DEDEDEDEDE

SEQ ID NO: 300 - protein, 15 aa, synthetic construct
EDEDEEDEDEEDEDE

SEQ ID NO: 301 - protein, 12 aa, synthetic construct
KSKSKSKSKSKS

SEQ ID NO: 302 - protein, 12 aa, synthetic construct
RKRKRKRKRKRK

SEQ ID NO: 303 - protein, 15 aa, synthetic construct
KRKRKKRKRKKRKRK

SEQ ID NO: 304 - protein, 9 aa, synthetic construct
GGSGGSGGS

SEQ ID NO: 305 - protein, 4 aa, synthetic construct
LVPR

SEQ ID NO: 306 - protein, 8 aa, synthetic construct
HHHHHHHH

(19) United States
(12) Patent Application Publication
(10) Pub. No.: US 2025/0188139 A1
(43) Pub. Date: Jun. 12, 2025

(54) VEGF-C MUTEINS FOR SELECTIVE LYMPHATIC STIMULATION

(71) Applicant: YALE UNIVERSITY, New Haven, CT (US)

(72) Inventors: Eric SONG, New Haven, CT (US); Akiko IWASAKI, Guilford, CT (US); Aaron RING, Milford, CT (US); Ting ZHOU, Hangzhou, Zhejiang Province (CN)

(73) Assignee: YALE UNIVERSITY, New Haven, CT (US)

(21) Appl. No.: 18/844,670

(22) PCT Filed: Mar. 8, 2023
(86) PCT No.: PCT/US2023/063952

§ 371 Date: Sep. 6, 2024

Related U.S. Application Data
(60) Provisional application No. 63/317,614, filed on Mar. 8, 2022.

(51) Int. Cl. C07K 14/475; A61K 9/00; A61K 38/00; A61K 45/06; A61P 27/06; C12N 15/86; C40B 40/02

(52) U.S. Cl. CPC C07K 14/475; A61K 9/0048; A61K 45/06; A61P 27/06; C12N 15/86; C40B 40/02; A61K 38/00; C07K 2319/30

(57) ABSTRACT

The present invention provides VEGF-C muteins having selective binding for VEGFR-3 over VEGFR-2. The present invention also provides method of inducing lymphangiogenesis in a subject in need thereof by administering to the subject an effective amount of a disclosed VEGF-C mutein. The present invention also provides methods for treating a disease or condition (e.g., cancer) in a subject in need thereof by administering to the subject an effective amount of a disclosed VEGF-C mutein.

### CROSS-REFERENCE TO RELATED APPLICATION

This application claims benefit of Provisional U.S. Application No. 63/317,614, filed Mar. 8, 2022, the contents of which is incorporated by reference in its entirety for all purposes.

### STATEMENT REGARDING FEDERALLY SPONSORED RESEARCH

This invention was made with government support under CA239444 awarded by National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Mar. 8, 2023, is named 251609_000089_SL.xml and is 347,984 bytes in size.

### FIELD OF THE INVENTION

The present invention relates generally to vascular endothelial growth factor C (VEGF-C) muteins having selective binding for vascular endothelial growth factor receptor-3 (VEGFR-3) over vascular endothelial growth factor receptor-2 (VEGFR-2). The present invention relates also to a method of inducing lymphangiogenesis in a subject in need thereof by administering to the subject an effective amount of a VEGF-C mutein or a functional fragment thereof. The present invention relates also to a method for treating a cancer in a subject in need thereof, the method comprising administering to the subject an effective amount of a VEGF-C mutein or a functional fragment thereof.

### BACKGROUND

Immune surveillance against pathogens and tumors in the central nervous system is thought to be limited owing to the lack of lymphatic drainage. It has been shown that the meningeal lymphatic vasculature can be manipulated to mount better immune responses against brain tumors. The immunity that is mediated by CD8 T cells to the glioblastoma antigen is very limited when the tumor is confined to the central nervous system, resulting in uncontrolled tumor growth. However, ectopic expression of vascular endothelial growth factor C (VEGF-C) promotes enhanced priming of CD8 T cells in the draining deep cervical lymph nodes, migration of CD8 T cells into the tumor, rapid clearance of the glioblastoma and a long-lasting antitumor memory response. Furthermore, transfection of an mRNA construct that expresses VEGF-C works synergistically with checkpoint blockade therapy to eradicate existing glioblastoma.

Vascular endothelial growth factor C (VEGF-C) binds to vascular endothelial growth factor receptor-2 (VEGFR-2) promoting the growth of blood vessels (angiogenesis) and regulating vascular permeability, and to vascular endothelial growth factor receptor-3 (VEGFR-3) promoting the growth of lymphatic vessels (lymphangiogenesis). VEGF-C acts on lymphatic endothelial cells (LECs) primarily via VEGFR-3 promoting survival, growth and migration. Angiogenesis is a necessity for growth of both primary tumors and metastases. There exists a need to reduce unwanted angiogenesis while promoting lymphangiogenesis.

### SUMMARY OF THE INVENTION

Various non-limiting aspects and embodiments of the invention are described below.

In one aspect, provided herein is an isolated vascular endothelial growth factor C (VEGF-C) mutein protein or a functional fragment thereof, wherein the VEGF-C mutein protein or functional fragment thereof has a reduced or no ability to stimulate blood endothelial cell proliferation, as compared to a wild-type VEGF-C protein from the same species but preserves the ability to stimulate lymphatic endothelial cell proliferation.

In one aspect, provided herein is an isolated vascular endothelial growth factor C (VEGF-C) mutein protein or a functional fragment thereof, wherein the VEGF-C mutein protein or functional fragment thereof (i) has a reduced binding affinity to vascular endothelial growth factor receptor-2 (VEGFR-2) as compared to a wild-type VEGF-C protein from the same species, (ii) has the ability to bind and generate signaling through vascular endothelial growth factor receptor-3 (VEGFR-3), and (iii) comprises an amino acid sequence that is at least 70% identical to the amino acid sequence of the wild-type VEGF-C protein from the same species.

In some embodiments of any of the above methods, the VEGF-C mutein protein or functional fragment thereof generates reduced or no signaling through VEGFR-2 as compared to the wild-type VEGF-C protein from the same species. In some embodiments of any of the above methods, the VEGF-C mutein protein or functional fragment thereof does not generate signaling through VEGFR-2. In some embodiments of any of the above methods, the signaling through VEGFR-2 is determined by measuring VEGFR-2-dependent AKT-phosphorylation and/or ERK-phosphorylation level in blood endothelial cells, by a wound healing assay, by a proliferation assay, or by an angiogenesis assay. In some embodiments of any of the above methods, the signaling through VEGFR-3 is determined by measuring VEGFR-3-dependent AKT-phosphorylation and/or ERK-phosphorylation level in lymphatic endothelial cells, by a wound healing assay (scratch assay), by a proliferation assay, or by an angiogenesis assay.

In some embodiments of any of the above methods, the VEGF-C mutein protein or functional fragment thereof does not induce angiogenesis.

In some embodiments of any of the above methods, the VEGF-C mutein protein is a mutein of a wild-type VEGF-C protein comprising amino acids 111-211 of SEQ ID NO: 4, or a polypeptide defined by the corresponding positions at the wild-type VEGF-C protein of another species. In some embodiments of any of the above methods, the wild-type VEGF-C protein comprises amino acids 111-211 of SEQ ID NO: 4.

In some embodiments of any of the above methods, the VEGF-C mutein protein or functional fragment thereof comprises one or more mutations selected from mutations at residues T112, L115, D119, Q126, T144, N145, K149, N163, S164, E165, I184, V186, L188, and P192, wherein the positions of said residues are defined in relation to SEQ ID NO: 4, or mutations at the corresponding residues within the wild-type VEGF-C protein of another species, or one or more mutations at residues T116, L119, D123, Q130, T148, N149, K153, N167, S168, E169, I188, V190, L192, and P196 wherein the positions of said residues are defined in relation to SEQ ID NO: 1.

In some embodiments of any of the above methods, the VEGF-C mutein protein or functional fragment thereof further comprises a mutation at residue C133, wherein the position of said residue is defined in relation to SEQ ID NO: 4, or a mutation at the corresponding residue within the wild-type VEGF-C protein of another species. In some embodiments, the mutation at residue C133 is C133A mutation.

In some embodiments of any of the above methods, the VEGF-C mutein protein is a mutein of a wild-type VEGF-C protein comprising amino acids 115-215 of SEQ ID NO: 1, or a polypeptide defined by the corresponding positions within the wild-type VEGF-C protein of another species. In some embodiments, the wild-type VEGF-C protein comprises amino acids 115-215 of SEQ ID NO: 1.

In some embodiments of any of the above methods, the VEGF-C mutein protein or functional fragment thereof comprises one or more mutations selected from mutations at residues T116, L119, D123, Q130, T148, N149, K153, N167, S168, E169, I188, V190, L192, and P196 wherein the positions of said residues are defined in relation to SEQ ID NO: 1.

In some embodiments of any of the above methods, the VEGF-C mutein protein or functional fragment thereof further comprises a mutation at residue C137, wherein the position of said residue is defined in relation to SEQ ID NO: 1, or mutations at the corresponding residues within the wild-type VEGF-C protein of other species. In some embodiments, the mutation at residue C137 is C137A mutation.

In some embodiments of any of the above methods, the mutation at residue L119 is L119E mutation, or L119M mutation; the mutation at residue D123 is D123N mutation; the mutation at residue Q130 is Q130K mutation; the mutation at residue N167 is N167R mutation, N167I mutation, N167Q mutation, or N167H mutation; the mutation at residue S168 is S168G mutation, or S168R mutation; the mutation at residue V190 is V190T mutation; and/or the mutation at residue L192 is L192I mutation.

In some embodiments of any of the above methods, the VEGF-C mutein protein or functional fragment thereof comprises one or more mutations selected from mutations at residues N167, S168, and/or L192.

In some embodiments of any of the above methods, the mutation at residue N167 is N167I mutation, N167Q mutation, or N167H mutation; the mutation at residue S168 is S168G mutation, or S168R mutation; and/or the mutation at residue L192 is L192I mutation.

In some embodiments of any of the above methods, the VEGF-C mutein protein or functional fragment thereof comprises N167Q mutation.

In some embodiments of any of the above methods, the VEGF-C mutein protein comprises the amino acid sequence of SEQ ID NO: 160 or SEQ ID NO: 56.

In some embodiments of any of the above methods, the VEGF-C mutein protein consists of the amino acid sequence of SEQ ID NO: 160 or SEQ ID NO: 56.

In some embodiments of any of the above methods, the VEGF-C mutein protein or functional fragment thereof comprises N167Q mutation and S168G mutation.

In some embodiments of any of the above methods, the VEGF-C mutein protein comprises the amino acid sequence of SEQ ID NO: 161 or SEQ ID NO: 57.

In some embodiments of any of the above methods, the VEGF-C mutein protein consists of the amino acid sequence of SEQ ID NO: 161 or SEQ ID NO: 57.

In some embodiments of any of the above methods, the VEGF-C mutein protein or functional fragment thereof comprises N167Q mutation and L192I mutation.

In some embodiments of any of the above methods, the VEGF-C mutein protein comprises the amino acid sequence of SEQ ID NO: 162 or SEQ ID NO: 58.

In some embodiments of any of the above methods, the VEGF-C mutein protein consists of the amino acid sequence of SEQ ID NO: 162 or SEQ ID NO: 58.

In some embodiments of any of the above methods, the VEGF-C mutein protein or functional fragment thereof comprises N167Q mutation, S168G mutation, and L192I mutation.

In some embodiments of any of the above methods, the VEGF-C mutein protein comprises the amino acid sequence of SEQ ID NO: 163 or SEQ ID NO: 59.

In some embodiments of any of the above methods, the VEGF-C mutein protein consists of the amino acid sequence of SEQ ID NO: 163 or SEQ ID NO: 59.

In some embodiments of any of the above methods, the VEGF-C mutein protein or functional fragment thereof comprises N167I mutation.

In some embodiments of any of the above methods, the VEGF-C mutein protein comprises the amino acid sequence of SEQ ID NO: 166 or SEQ ID NO: 62.

In some embodiments of any of the above methods, the VEGF-C mutein protein consists of the amino acid sequence of SEQ ID NO: 166 or SEQ ID NO: 62.

In some embodiments of any of the above methods, the VEGF-C mutein protein or functional fragment thereof comprises N167I mutation and S168G mutation.

In some embodiments of any of the above methods, the VEGF-C mutein protein comprises the amino acid sequence of SEQ ID NO: 167 or SEQ ID NO: 63.

In some embodiments of any of the above methods, the VEGF-C mutein protein consists of the amino acid sequence of SEQ ID NO: 167 or SEQ ID NO: 63.

In some embodiments of any of the above methods, the VEGF-C mutein protein or functional fragment thereof comprises N167I mutation and L192I mutation.

In some embodiments of any of the above methods, the VEGF-C mutein protein comprises the amino acid sequence of SEQ ID NO: 168 or SEQ ID NO: 64.

In some embodiments of any of the above methods, the VEGF-C mutein protein consists of the amino acid sequence of SEQ ID NO: 168 or SEQ ID NO: 64.

In some embodiments of any of the above methods, the VEGF-C mutein protein or functional fragment thereof comprises N167I mutation, S168G mutation, and L192I mutation.

In some embodiments of any of the above methods, the VEGF-C mutein protein comprises the amino acid sequence of SEQ ID NO: 169 or SEQ ID NO: 65.

In some embodiments of any of the above methods, the VEGF-C mutein protein consists of the amino acid sequence of SEQ ID NO: 169 or SEQ ID NO: 65.

In some embodiments of any of the above methods, the VEGF-C mutein protein or functional fragment thereof comprises S168G mutation.

In some embodiments of any of the above methods, the VEGF-C mutein protein comprises the amino acid sequence of SEQ ID NO: 172 or SEQ ID NO: 68.

In some embodiments of any of the above methods, the VEGF-C mutein protein consists of the amino acid sequence of SEQ ID NO: 172 or SEQ ID NO: 68.

In some embodiments of any of the above methods, the VEGF-C mutein protein or functional fragment thereof comprises S168G mutation and L192I mutation.

In some embodiments of any of the above methods, the VEGF-C mutein protein comprises the amino acid sequence of SEQ ID NO: 174 or SEQ ID NO: 70.

In some embodiments of any of the above methods, the VEGF-C mutein protein consists of the amino acid sequence of SEQ ID NO: 174 or SEQ ID NO: 70.

In some embodiments of any of the above methods, the VEGF-C mutein protein or functional fragment thereof comprises N167H mutation.

In some embodiments of any of the above methods, the VEGF-C mutein protein comprises the amino acid sequence of SEQ ID NO: 184 or SEQ ID NO: 80.

In some embodiments of any of the above methods, the VEGF-C mutein protein consists of the amino acid sequence of SEQ ID NO: 184 or SEQ ID NO: 80.

In some embodiments of any of the above methods, the VEGF-C mutein protein or functional fragment thereof comprises N167I mutation and S168R mutation.

In some embodiments of any of the above methods, the VEGF-C mutein protein comprises the amino acid sequence of SEQ ID NO: 192 or SEQ ID NO: 88.

In some embodiments of any of the above methods, the VEGF-C mutein protein consists of the amino acid sequence of SEQ ID NO: 192 or SEQ ID NO: 88.

In one aspect, provided herein is a fusion protein or conjugate comprising the VEGF-C mutein protein or functional fragment thereof disclosed herein, wherein the mutein protein or a functional fragment thereof, is fused and/or conjugated to one of more heterologous moieties.

In some embodiments, the one of more heterologous moieties are selected from an immunoglobulin or a functional fragment thereof, an albumin or a functional fragment thereof, an albumin-binding antibody or a functional fragment thereof, and a polyethylene glycol (PEG) polymer.

In some embodiments, the immunoglobulin or functional fragment thereof comprises an IgG Fc domain. In some embodiments, the IgG Fc domain is modified to reduce a Fc effector function. In some embodiments, the IgG Fc domain comprises a mutation at residue N297. In some embodiments, the mutation at residue N297 is selected from N297Q, N297A and N297G.

In one aspect, provided herein is an isolated polynucleotide molecule encoding the VEGF-C mutein protein or functional fragment thereof of the present disclosure or the fusion proteins of the present disclosure.

In some embodiments, the polynucleotide molecule comprises the nucleotide sequence of SEQ ID NO: 210 or SEQ ID NO: 108. In some embodiments, the polynucleotide molecule comprises the nucleotide sequence of SEQ ID NO: 211 or SEQ ID NO: 109. In some embodiments, the polynucleotide molecule comprises the nucleotide sequence of SEQ ID NO: 212 or SEQ ID NO: 110. In some embodiments, the polynucleotide molecule comprises the nucleotide sequence of SEQ ID NO: 213 or SEQ ID NO: 111. In some embodiments, the polynucleotide molecule comprises the nucleotide sequence of SEQ ID NO: 216 or SEQ ID NO: 114. In some embodiments, the polynucleotide molecule comprises the nucleotide sequence of SEQ ID NO: 217 or SEQ ID NO: 115. In some embodiments, the polynucleotide molecule comprises the nucleotide sequence of SEQ ID NO: 218 or SEQ ID NO: 116. In some embodiments, the polynucleotide molecule comprises the nucleotide sequence of SEQ ID NO: 219 or SEQ ID NO: 117. In some embodiments, the polynucleotide molecule comprises the nucleotide sequence of SEQ ID NO: 222 or SEQ ID NO: 120. In some embodiments, the polynucleotide molecule comprises the nucleotide sequence of SEQ ID NO: 224 or SEQ ID NO: 122. In some embodiments, the polynucleotide molecule comprises the nucleotide sequence of SEQ ID NO: 234 or SEQ ID NO: 132. In some embodiments, the polynucleotide molecule comprises the nucleotide sequence of SEQ ID NO: 242 or SEQ ID NO: 140. In some embodiments, the polynucleotide molecule comprises a nucleotide sequence encoding the VEGF-C mutein protein or functional fragment thereof which is operably linked to a promoter.

In some embodiments, the polynucleotide molecule is an mRNA.

In some embodiments, the polynucleotide molecule comprises one or more nucleotide modifications. In some embodiments, the one or more nucleotide modifications are a 5′cap, a 5-methylcytosine, or a pseudo-uridine.

In one aspect, provided herein is a vector comprising the polynucleotide molecules of the present disclosure.

In some embodiments, the vector is a viral vector. In some embodiments, the viral vector is derived from a herpes virus, a cytomegalovirus, a poliovirus, an alphavirus, a vaccinia virus, a rabies virus, an adeno-associated virus (AAV), a retrovirus, a lentivirus, or an adenovirus.

In one aspect, provided herein is a particle comprising the polynucleotide molecules of the present disclosure. In some embodiments, the particle is a nanoparticle, lipid particle, microparticle, lipid nanoparticle, polymer particle, or virus-like particle (VLP).

In one aspect, provided herein is a host cell comprising the polynucleotides of the present disclosure or the vectors of the present disclosure.

In one aspect, provided herein is a method of producing a VEGF-C mutein protein or functional fragment thereof, or fusion protein thereof, comprising culturing the host cell of the present disclosure under conditions at which the VEGF-C mutein protein or functional fragment thereof, or fusion protein thereof is expressed.

In one aspect, provided herein is a VEGF-C mutein protein or functional fragment thereof, or fusion protein thereof, produced by the methods of the present disclosure.

In one aspect, provided herein is a kit comprising the VEGF-C mutein proteins or functional fragments thereof of the present disclosure or the fusion proteins or conjugates of the present disclosure, and optionally instructions for use.

In one aspect, provided herein is a kit comprising the polynucleotides of the present disclosure or the vectors of the present disclosure, or the particles of the present disclosure, and optionally instructions for use.

In one aspect, provided herein is a pharmaceutical composition comprising a VEGF-C mutein protein or functional fragment thereof of the present disclosure or the fusion protein or conjugate of the present disclosure, or the polynucleotide molecule of the present disclosure, or the vector of the present disclosure, or the particle of the present disclosure, and a pharmaceutically acceptable carrier or diluent.

In some embodiments, the composition comprises mRNA encoding the VEGF-C mutein protein or functional fragment thereof, or fusion protein thereof as mRNA-nanoparticle formulation.

In some embodiments, the pharmaceutical composition further comprising an immunotherapeutic agent. In some embodiments, the immunotherapeutic agent is an immune checkpoint inhibitor. In some embodiments, the immune checkpoint inhibitor targets PD-1, PD-L1, CTLA-4, TIGIT, TIM-3, LAG-3, BTLA, GITR, 4-1BB, or Ox-40. In some embodiments, the immune checkpoint inhibitor is an anti-PD-1 antibody, an anti-PD-L1 antibody, an anti-CTLA-4 antibody, an anti-TIGIT antibody, an anti-TIM-3 antibody, an anti-LAG-3 antibody, an anti-BLTA antibody, an anti-GITR antibody, an anti-4-IBB antibody, or an anti-Ox-40 antibody.

In some embodiments, the pharmaceutical composition is formulated for intrathecal administration. In some embodiments, the pharmaceutical composition is formulated for intratumoral administration. In some embodiments, the pharmaceutical composition is formulated for systemic administration. In some embodiments, the pharmaceutical composition is formulated for intracisternal administration. In some embodiments, the pharmaceutical composition is formulated for eye-drop administration. In some embodiments, the pharmaceutical composition is formulated for intraocular administration.

In one aspect, provided herein is a method of inducing lymphangiogenesis in a subject in need thereof, the method comprising administering to the subject an effective amount of the VEGF-C mutein proteins or functional fragments thereof of the present disclosure or the fusion proteins or conjugate of the present disclosure, or the polynucleotide molecules of the present disclosure, or the vectors of the present disclosure, or the particles of the present disclosure, or the pharmaceutical compositions of the present disclosure.

In some embodiments of any of the above methods, the administration said VEGF-C mutein protein or functional fragment thereof, fusion protein, conjugate, polynucleotide molecule, vector, particle, or pharmaceutical composition, does not cause one or more side effects associated with administration of a wild-type VEGF-C protein, or corresponding fusion protein, conjugate, polynucleotide molecule, vector, particle, or pharmaceutical composition. In some embodiments, the one or more side effects are angiogenesis and/or increased intraocular pressure (IOP).

In some embodiments of any of the above methods, the VEGF-C mutein protein or functional fragment thereof, fusion protein, conjugate, polynucleotide molecule, vector, particle, or pharmaceutical composition is administered intrathecally, intraocularly, intratumorally, intracisternally, intravitreally, via eye drops, subcutaneously, intradermally, via inhalation, via long-dwelling catheter, orally, topically, or systemically.

In some embodiments of any of the above methods, the VEGF-C mutein protein or functional fragment thereof, fusion protein, conjugate, polynucleotide molecule, vector, particle, or pharmaceutical composition is administered to the cisterna magna or directly into the lymphatic system.

In some embodiments of any of the above methods, the subject has a disease or condition selected from cancer, coronary vessel function, osmoregulation, heart ischemia, restenosis, fibrosis, colitis, chronic liver disease, polycystic kidney disease, diseases or conditions associated with lymph node transplant, Alzheimer's disease, Parkinson's disease, stroke, cerebral ischemia, wound healing, lymphedema, Hennekam syndrome, Milroy's disease, Turner syndrome, age related macular degeneration, glaucoma, central serous chorioretinopathy, diabetic retinopathy, macular edema and retinal edema.

In some embodiments of any of the above methods, the diseases or conditions associated with lymph node transplant are breast cancer associated lymphedema, idiopathic lymphedema, and/or heart failure associated lymphedema.

In one aspect, provided herein is a method of treating a disease or condition in a subject in need thereof, the method comprising administering to the subject an effective amount of a VEGF-C mutein proteins or functional fragments thereof of the present disclosure or the fusion proteins or conjugate of the present disclosure, or the polynucleotide molecules of the present disclosure, or the vectors of the present disclosure, or the particles of the present disclosure, or the pharmaceutical compositions of the present disclosure.

In some embodiments, wherein the method provided herein is a method of treating a disease or condition in a subject in need thereof, the disease or condition is cancer, coronary vessel function, osmoregulation, heart ischemia, restenosis, fibrosis, colitis, chronic liver disease, polycystic kidney disease, diseases or conditions associated with lymph node transplant, Alzheimer's disease, Parkinson's disease, stroke, cerebral ischemia, wound healing, lymphedema, Hennekam syndrome, Milroy's disease, Turner syndrome, age related macular degeneration, glaucoma, central serous chorioretinopathy, diabetic retinopathy, macular edema and retinal edema. In some embodiments, the cancer is melanoma, lung cancer, breast cancer, stomach cancer, esophageal cancer, ovarian cancer, uterine cancer, cervical cancer, head and neck squamous cell carcinoma, thyroid cancers, liquid cancer, kidney cancers, urothelial bladder cancers, prostate cancers, pheochromocytoma, cholangiocarcinoma, liver hepatocellular carcinoma, pancreatic ductal adenocarcinoma, thymoma, sarcoma, mesothelioma, testicular cancer, or colorectal cancer. In some embodiments, the cancer is in the brain or the central nervous system of the subject. In some embodiments, the cancer is selected from glioma, ependymoma, subependymoma, primitive neuroectodermal tumor, ganglioglioma, Schwannoma, germinoma, craniopharyngioma, meningioma, CNS lymphoma, pineal tumor, retinoblastoma, uveal melanoma and rhabdoid tumor.

In some embodiments, the method of treating a disease or condition in a subject in need thereof further comprises administering an immunotherapeutic agent. In some embodiments, the immunotherapeutic agent is an immune checkpoint inhibitor. In some embodiments, the immune checkpoint inhibitor targets PD-1, PD-L1, CTLA-4, TIGIT, TIM-3, LAG-3, BTLA, GITR, 4-1BB, or Ox-40. In some embodiments, the immune checkpoint inhibitor is an anti-PD-1 antibody, an anti-PD-L1 antibody, an anti-CTLA-4 antibody, an anti-TIGIT antibody, an anti-TIM-3 antibody, an anti-LAG-3 antibody, an anti-BLTA antibody, an anti-GITR antibody, an anti-4-IBB antibody, or an anti-Ox-40 antibody. In some embodiments, the VEGF-C mutein protein or functional fragment thereof, fusion protein, conjugate, polynucleotide molecule, vector, particle, or pharmaceutical composition is administered intrathecally, intraocularly, intratumorally, intracisternally, intravitreally, via eye drops, subcutaneously, intradermally, via inhalation, via long-dwelling catheter, orally, topically, or systemically. In some embodiments, the VEGF-C mutein protein or functional fragment thereof, fusion protein, conjugate, polynucleotide molecule, vector, particle, or pharmaceutical composition is administered to the cisterna magna or directly into the lymphatic system.

In some embodiments, wherein the method provided herein is a method of treating a disease or condition in a subject in need thereof, and wherein the disease is cancer, the method further comprising administering an additional anti-cancer treatment to the subject. In some embodiments, the additional anti-cancer treatment is selected from surgery, radiation therapy, administration of a chemotherapeutic agent, an immunotherapy, and any combinations thereof.

In one aspect, provided herein is a method for modulating intraocular pressure in a subject in need thereof comprising administering to the subject an effective amount of the VEGF-C mutein protein or functional fragment thereof, the fusion protein or conjugate, the polynucleotide molecule, the vector, the particle, or the pharmaceutical composition of the present disclosure, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above. In some embodiments, the VEGF-C mutein protein or the corresponding wild-type VEGF-C protein, or functional fragment thereof, fusion protein, conjugate, polynucleotide molecule, vector, particle, or pharmaceutical composition, or the corresponding wild-type VEGF-C protein or functional fragment thereof, or the fusion protein or conjugate thereof, or the polynucleotide molecule encoding said wild-type VEGF-C protein or functional fragment thereof, or the vector or particle comprising said polynucleotide molecule, or the pharmaceutical composition comprising any of the above, is administered to the posterior eye. In some embodiments, the administration is intraocular. In some embodiments, the intraocular administration is intravitreal, via eye drops, or subretinal.

In one aspect, provided herein is a method for removing unwanted fluid in an eye of a subject in need thereof comprising administering to the subject an effective amount of the VEGF-C mutein protein or functional fragment thereof, the fusion protein or conjugate, the polynucleotide molecule, the vector, the particle, or the pharmaceutical composition of the present disclosure, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above. In some embodiments, the unwanted fluid is optic nerve, retinal, subretinal, choroidal, or suprachoroidal fluid. In some embodiments, the subject has glaucoma, macular edema, central serous chorioretinopathy, retinal edema, papilledema, macular degeneration, or diabetic retinopathy. In some embodiments, the administration is intraocular. In some embodiments, the intraocular administration is intravitreal, via eye drops, or subretinal.

In one aspect, provided herein is a method for providing neuroprotection in a subject in need thereof comprising administering to the subject an effective amount of the VEGF-C mutein protein or functional fragment thereof, the fusion protein or conjugate, the polynucleotide molecule, or the vector, the particle, or the pharmaceutical composition of the present disclosure, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above.

In one aspect, provided herein is a vaccine comprising the VEGF-C mutein protein or functional fragment thereof, the fusion protein or conjugate, the polynucleotide molecule, the vector, the particle, the pharmaceutical composition of the present disclosure.

In one aspect, provided herein is a method inducing an immune response in a subject in need thereof in a subject in need thereof, the method comprising administering to the subject an effective amount of the vaccine of the present disclosure.

In some embodiments of any of the above methods, the subject is a human.

In a further aspect, provided herein is a method of generating a library of VEGF-C muteins having selective binding to VEGFR-3, wherein amino acid residues comprising the shared binding interface on VEGF-C which binds both VEGFR-3 and VEGFR-2 are diversified to other amino acids via mutagenesis of the corresponding nucleic acid sequence.

In a further aspect, provided herein is a yeast cell library for selection of VEGF-C mutein proteins, comprising a plurality of yeast cells comprising a cell wall peptide anchor sequence, a linker peptide, and the VEGF-C mutein sequence.

These and other aspects described herein will be apparent to those of ordinary skill in the art in the following description, claims and drawings.

### BRIEF DESCRIPTION OF THE DRAWINGS

FIGS. 1A-1B depict a summary of VEGF-C pleiotropism. FIG. 1A is a schematic showing that VEGF-C binds to both VEGFR-2 and VEGFR-3, promoting angiogenesis and lymphangiogenesis, respectively. FIG. 1B is a schematic of designed VEGF-C mutein showing specific binding to VEGFR-3 with loss of binding to VEGFR-2.

FIGS. 2A-2B depict VEGF-C display construct design for dimerization and screening. FIG. 2A shows multiple methods to display VEGF-C on yeast surface which were utilized to optimize VEGF-C dimerization on the surface. Final resulting combination displayed wild type VEGF-C with high binding affinity to VEGFR-3 and lower affinity towards VEGFR-2. FIG. 2B is a schematic showing yeast-display library selection towards finding a VEGFR-3 specific VEGF-C mutein.

FIG. 3 depicts VEGF-C mutein library design. Structure guided primer libraries were designed for generation of ˜103 VEGF-C mutein combinations. Structures of VEGF-C binding to VEGFR-3 and VEGFR-2 were studied to identify key residues on the surface of binding sites. Amino acids that can have significant polarization changes in these residues were chosen for designing of primers. Figure discloses SEQ ID NOs: 27-34 and 34-39, respectively, in order of appearance.

FIG. 4 depicts that VEGF-C muteins were positively selected for VEGFR-3 and negatively selected for VEGFR-2 binding. Wild type VEGF-C showed binding towards yeast cells expressing both VEGFR-2 and VEGFR-3 (top row, WT). First two rounds selected for binding towards VEGFR-3. In subsequent rounds, decreasing amounts of VEGFR-3 were utilized to select for high binding affinity muteins towards VEGFR-3. At the same time, VEGFR-2 was included and negatively selected against in order to enrich for muteins that lost binding affinity towards VEGFR-2 with high binding to VEGFR-3.

FIG. 5 depicts confirmation of receptor affinity preference post final rounds of selection. Muteins that were selected for positively for VEGFR-3 and negatively against VEGFR-2 display high binding for VEGFR-3 and no binding towards VEGFR-2 even at concentrations of the receptor 100× higher than wild type binding.

FIG. 6 depicts confirmation of receptor affinity preference post final rounds of selection. Clonal muteins after round 5 and 6 of selection were sequenced to identify unique clones that were enriched for in the mutein population. Unique mutein residues were identified to create specific mutations into wild type VEGF-C for validation.

FIG. 7 depicts summary of sequences, rate of mutation, and affinity for selected murine clones and final LS-VEGF-C. Position and identity of key residues are listed in the first two rows. Shaded columns indicate mutations at contact positions, all of which inform the final mutations in WT-VEGF-C that create LS-VEGF-C. Rate of mutation for clones and at specific residue positions is also indicated. Affinity to VEGFR-2 and VEGFR-3 was determined by SPR for WT- and LS-VEGF-C.

FIG. 8 depicts VEGFR-2 and VEGFR-3 affinities of WT VEGF-C and after round 6 of selection.

FIG. 9 depicts that isolated VEGF-C muteins displayed specific signaling through VEGFR-3 in vitro. VEGF-A, wild type VEGF-C and VEGF-C mutein (163I and RTI) signaling was evaluated in vitro using human umbilical vein endothelial cells (HUVEC) and human dermal lymphatic endothelial cells (juvenile HUVEC-j and adult HUVEC-a). HUVECs specifically express VEGFR-2 while HDLECs express both VEGFR-2 and VEGFR-3, allowing for evaluation of each receptor signaling. Cells were stimulated with each protein and protein lysates were collected for western blots to detect ERK phosphorylation (downstream of VEGFR-2 and VEGFR-3).

FIG. 10 depicts use of VEGF-C mutein in vivo. Single administration of VEGF-C muteins intravitreally resulted in sustained decrease in intraocular pressure (IOP) in a wild type mouse compared with its counterpart.

FIG. 11 depicts use of VEGF-C mutein in vivo. Wild type VEGF-C and VEGF-C muteins were evaluated in vivo for the treatment of brain tumors. In combination with anti-PD-1 antibodies, VEGF-C muteins showed significant therapeutic benefits treating brain tumors.

FIG. 12 depicts VEGFR-2 and VEGFR-3 affinities of WT VEGF-C and previously established VEGFR-3 specific ligand, VEGF-C152S/C-156S.

FIG. 13 depicts that isolated VEGF-C muteins displayed specific signaling through VEGFR-3 in vivo. Wild type VEGF-C and VEGF-C mutein (RTI) signaling was evaluated in vivo. These vectors were injected into eyes of mice. 24 hours later, eyes were enucleated and made into single cell suspensions to check for AKT-phosphorylation. Wild type VEGF-C retained signaling through VEGFR-2 in blood endothelial cells while mutant VEGF-C(RTI) no longer had signaling. Both the wild type protein and RTI mutein signaled through VEGFR-3 in lymphatic endothelial cells.

FIG. 14 shows that routes of administration resulted in differential drops in IOP in vivo. VEGF-C or VEGF-C mutein (RTI) was administered either by eye drops, injection into the anterior chamber (AC) or intravitreally. While in the AC, both wild type and the mutein had similar activity, the mutein showed selective ability to decrease eye pressure when administered via eyedrops or intravitreally.

FIGS. 15A-15D show directed evolution of VEGF-C variants for lymphatic-specific VEGFR-3 binding. FIG. 15A shows mVEGFR2 affinity of WT VEGF-C and muteins after round 6 of selection iteration. FIG. 15B shows mVEGFR3 affinity of WT VEGF-C and muteins after round 6 of selection iteration. FIG. 15C shows downstream ERK phosphorylation of HUVEC (R2+) after administration of control (n=3), VEGF-A (n=3), WT VEGF-C (n=3), and LS-VEGF-C (n=3). FIG. 15D shows proliferation of HUVEC (R2+) and HDLEC (R2+R3+) after administration of control (n=3), VEGF-A (n=3), WT VEGF-C (n=3), and LS-VEGF-C (n=3). In FIGS. 15C and 15D, data shown as mean±s.e.m., with *P<0.05, **P<0.01 ***P<0.005, ****P<0.001, ns being nonsignificant.

FIGS. 16A-16B show that administration of LS-VEGF-C results in decrease of lymphedema in a mouse model of lymphedema. LS-VEGF-C was administered subcutaneously in mice near the cite of lymphatic ligation and compared to control resulted in significant decrease in ankle size. Mice were treated twice to demonstrate the redosing capability of LS-VEGF-C. FIG. 16C-16D Tumor bearing mice were treated with tumor vaccines and PD-1 along with LS-VEGF-C or WT-VEGF-C. FIG. 16C demonstrates the tumor growth curves while FIG. 16D demonstrates the mice survivability.

FIGS. 17A-17E show that LS-VEGF-C leads to lymphangiogenic changes, without increasing vascular permeability, in the eye. FIG. 17A shows intraocular pressure measurements after topical (eyedrop), intracameral, and intravitreal administration of WT-VEGF-C in WT mice. FIG. 17B shows intraocular pressure measurements after topical (eyedrop), intracameral, and intravitreal administration of LS-VEGF-C in WT mice. FIG. 17C shows representative fundus (left column), fluorescent angiogenic (middle column), and OCT (right column) images after intravitreal administration of VEGF-A, WT-VEGF-C, and LS-VEGF-C. FIG. 17D shows normalized measurements of fluorescent intensity across the fluorescein angiographs in 2, which indicate permeability of blood vessels into the extravascular space; sharp peaks above baseline (normalized value of ˜20) demonstrate presence of blood vessels with no leakage, and sustained elevated values indicate enhanced permeability. FIG. 17E shows repeated measure comparisons of the absorbance of systemically delivered Evans blue in the eye, indicating permeability of retinal blood vessels into the extravascular space, after IVT administration of PBS (control), VEGF-A, WT VEGF-C, and LS-VEGF-C. For FIG. 17E, ***P<0.005, ****P<0.001, *P<0.05, ns being nonsignificant.

FIGS. 18A-18H show that LS-VEGF-C provides neuroprotection in glaucoma models. FIG. 18A, is a schematic representation of the microbead mouse model of glaucoma and evaluation by IOP measurements. FIG. 18B shows a summary (mean±s.e.m., left) and individual IOP measurements (right) over 28 days for WT mice (Control, n=12, microbead mouse model of glaucoma (Induced Glaucoma, n=12), microbead mouse model of glaucoma with IVT LS-VEGF-C (Induced Glaucoma+LS-VEGF-C (IVT), n=12, microbead mouse model of glaucoma with intravitreally administered LS-VEGF-C (Induced Glaucoma+LS-VEGF-C (IVT), n=12, microbead mouse model of glaucoma with topically administered LS-VEGF-C (Induced Glaucoma+LS-VEGF-C (eyedrops), n=12, and WT mice with intravitreally administered LS-VEGF-C (LS-VEGF-C (IVT), n=12). FIG. 18C shows Brn3a and DAPI-labeled confocal images of retinas after intravitreally injected NMDA, excitotoxic study. WT mice (control), microbead glaucoma model (induced glaucoma), and microbead glaucoma model with LS-VEGF-C administered intravitreally (+LS-VEGF-C IVT) and topically (+LS-VEGF-C eye drops) were compared. FIG. 18D shows counts of Brn3a cells observed in image field (Control, n=7; microbead glaucoma model, n=6; microbead glaucoma model+IVT LS-VEGF-C, n=7; microbead glaucoma model+LS-VEGF-C eye drop, n=8). FIG. 18E is a schematic representation of the DBA2J pigment dispersion mouse model of glaucoma. FIG. 18F shows summary (mean #s.e.m., left) and individual IOP measurements (right) over 28 days for WT mice (Control, n=12), microbead mouse model of glaucoma (Induced Glaucoma, n=12, microbead mouse model of glaucoma with IVT LS-VEGF-C (Induced Glaucoma+LS-VEGF-C (IVT), n=12), microbead mouse model of glaucoma with intravitreally administered LS-VEGF-C (Induced Glaucoma+LS-VEGF-C (IVT), n=12), microbead mouse model of glaucoma with topically administered LS-VEGF-C (Induced Glaucoma+LS-VEGF-C (eyedrops), n=12), and WT mice with intravitreally administered LS-VEGF-C (LS-VEGF-C (IVT), n=12). FIG. 18G shows images of RGC axons in the optic nerve of control D2-Gpnmb+ mice and DBA2J mice with different treatments: untreated (spontaneous glaucoma), +LS-VEGF-C IVT, +LS-VEGF-C eyedrops. FIG. 18H shows counts of RGC axon cells observed in image field (Control, n=3; DBA2J glaucoma, n=4; LS-VEGF-C IVT, n=4; LS-VEGF-C eye drop, n=4).

FIG. 19 shows data from VEGFR2 and VEGFR3 binding assay of WT VEGF-C to VEGFR2 and VEGFR3.

FIGS. 20A-20F show that administration of LS-VEGF-C results in decrease of lymphedema in a mouse model of lymphedema. LS-VEGF-C was administered subcutaneously in mice near the cite of lymphatic ligation and compared to control resulted in significant decrease in ankle size. Mice were treated twice to demonstrate the redosing capability of LS-VEGF-C.

FIGS. 21A-21D show that LS-VEGF-C offers a unique mechanism of action with sustained effect observed. FIG. 21A shows IOP measurements (mean±s.e.m.) over 10 days after topical (left), intracameral (middle), and intravitreal (right) administration of WT-VEGF-C and LS-VEGF-C. FIG. 21B is a graphical representation with arrows indicating fluid flow into and out of the eye. FIG. 21C shows IOP measurements after topical administration of current FDA-approved drugs for glaucoma. Data point for first hour is excluded, due to confounding IOP-lowering effects of light anesthesia. FIG. 21D shows Comparison of IOP measurements (mean±s.e.m.) of control mice, mice treated with FDA-approved topical glaucoma drugs at time point when greatest IOP reduction is observed (Dorzolamide, Dz, at 2 hours; Rhopressa, Rho, at 2 hours; Brimonidine, Bri, at 2 hours; Latanoprost, Lat, at 3 hours; Timolol at 2 hours), 2 days post-IV injection of LS-VEGF-C, and topical administration of FDA-approved drugs at time point when greatest IOP reduction is observed with mice that were administered LS-VEGF-C intravitreally two days prior. Data shown as mean±s.e.m., with *P<0.05, **P<0.01 ***P<0.005, ****P<0.001, ns being nonsignificant.

FIGS. 22A-22D show images of anterior side of eye showing similar indications as their posterior counterpart. FIG. 22A shows images of control WT mice when focused on the cornea and the iris (n=3). FIG. 22B shows images of WT mice after IVT administration of VEGF-A when focused on the cornea and the iris (n=3). Neovasculature and corneal cloudiness was observed. FIG. 22C shows images of WT mice after IVT administration of WT-VEGF-C when focused on the cornea and the iris (n=3). Corneal cloudiness was observed. FIG. 22D shows images of WT mice after IVT administration of LS-VEGF-C when focused on the cornea and the iris (n=3).

FIGS. 23A-23B show that LS-VEGF-C provides neuroprotection in glaucoma models. FIG. 23A shows Brn3a and DAPI-labeled confocal images of retinas after intravitreally injected NMDA, excitotoxic study. FIG. 23B shows the number of RGC cells observed in image field from NMDA-facilitated excitatory study.

FIG. 24 shows VEGFR2 binding summary for hLS-VEGF-C variants and summarizes the binding of the hVEGF-C variants to hVEGFR2 compared to WT VEGF-C as expressed by the % of the yeast population that was positive for staining. Lower values are desired, indicating less VEGFR2 engagement.

FIG. 25 shows relative VEGFR3 vs VEGFR2 bias for LS-hVEGF-C clones. FIG. 25 contains plots of the binding of hVEGFR3 (as expressed by mean fluorescence intensity, MFI) versus hVEGFR2 binding (as expressed by the % of the yeast population that was positive for staining). For the top graph, 100 nM of VEGFR3 was used. For the bottom graph, 10 nM of hVEGFR3 was used. Selected points are labeled with the number of the LS-hVEGF-C mutein. Clones in the lower right quadrant of these plots are preferred, as they demonstrated biased binding to VEGFR3 versus VEGFR2.

FIG. 26 shows VEGFR2 and VEGFR3 binding for LS-hVEGF-C; the specific values of hVEGFR2 and hVEGFR3 binding for each clone that are depicted in FIGS. 24 and 25.

FIG. 27 depicts the binding isotherms of selected LS-hVEGF-C muteins for hVEGFR3. Specific EC50 values and R2 values for the curve fits are provided at the bottom of the FIG. 27.

FIGS. 28A-28M show quality of human VEGF-C muteins fused to human IgG1 Fc. FIG. 28A shows quality of human C137A clone fusion. FIG. 28B shows quality of human clone 7 fusion. FIG. 28C shows quality of human clone 8 fusion. FIG. 28D shows quality of human clone 9 fusion. FIG. 28E shows quality of human clone 10 fusion. FIG. 28F shows quality of human clone 13 fusion. FIG. 28G shows quality of human clone 14 fusion. FIG. 28H shows quality of human clone 15 fusion. FIG. 28I shows quality of human clone 19 fusion. FIG. 28K shows quality of human clone 21 fusion. FIG. 28L shows quality of human clone 31 fusion. FIG. 28M shows quality of human clone 39 fusion.

FIG. 29 shows the effect of and eye pressures on mice treated intravitreally with RTI-Fc mutein.

FIG. 30 shows intraocular pressure drops on mice treated with intravitreal LS-VEGF-C in the form of a monomer, with albumin conjugation or Fc conjugation.

### DETAILED DESCRIPTION

The present invention provides VEGF-C muteins and methods of inducing lymphangiogenesis in a subject in need thereof, the method comprising administering to the subject an effective amount of a VEGF-C mutein.

The terms “a,” “an,” and “the” do not denote a limitation of quantity, but rather denote the presence of “at least one” of the referenced items.

The term “about” when used before a numerical value indicates that the value may vary within a reasonable range, such as within ±10%, ±5% or ±1% of the stated value, and include the stated value.

The terms “patient,” “individual,” “subject,” “mammal,” and “animal” are used interchangeably herein and refer to mammals, including, without limitation, human and veterinary animals (e.g., cats, dogs, rabbits, cows, horses, sheep, pigs, etc.) and experimental animal models. In a preferred embodiment, the subject is a human.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the terms “mutein protein,” “mutein polypeptide” and “mutein” are used interchangeably to refer to a protein with an altered amino acid sequence as compared to its wild-type counterpart. Amino acid sequence alterations may comprise amino acid substitutions, deletions or additions.

As used herein, the term “lymphangiogenesis” refers to the process of the formation of lymphatic vessels and/or stimulation of lymphatic vasculature function.

In one aspect, provided herein is an isolated vascular endothelial growth factor C (VEGF-C) mutein protein or a functional fragment thereof, having selective binding for VEGFR-3 over VEGFR-2.

In some embodiments, the VEGF-C mutein protein or functional fragment thereof has a reduced or no ability to stimulate blood endothelial cell proliferation, as compared to a wild-type VEGF-C protein from the same species but preserves the ability to stimulate lymphatic endothelial cell proliferation.

In some embodiments, the VEGF-C mutein protein or functional fragment thereof (i) has a reduced binding affinity to VEGFR-2 as compared to a wild-type VEGF-C protein from the same species, (ii) has the ability to bind and generate signaling through VEGFR-3, and (iii) comprises an amino acid sequence that is at least 70% identical to the amino acid sequence of the wild-type VEGF-C protein from the same species.

In some embodiments, the VEGF-C mutein protein or functional fragment thereof has a binding affinity to VEGFR-2 that is reduced by about 10%, 20%, 30%, 40%, 50%, 60%, 70%, 80%, 90%, 95%, 98%, 99% or more as compared to a wild-type VEGF-C protein from the same species.

In some embodiments, the VEGF-C mutein protein or functional fragment thereof has an ability to bind and generate signaling through VEGFR-3 which is about the same, increased, or mildly reduced as compared to a wild-type VEGF-C protein from the same species.

In a cell, VEGF-C is produced as an inactive propeptide. Convertases such as furin, PC5, or PC7 cleave between the VEGF homology domain and the C-terminal silk homology domain generating pro-VEGF-C. Pro-VEGF-C is able to bind VEGFR-3 but does not activate it. The second proteolytic cleavage by A disintegrin and metalloproteinase with thrombospondin motifs 3 (ADAMTS3) removes both terminal domains resulting in mature active VEGF-C protein. See, e.g., Rauniyar et al., Front. Bioeng. Biotechnol., 2018, Vol. 6, Art. 7, doi.org/10.3389/fbioe.2018.00007. In case of the wild-type human VEGF-C, mature VEGF-C protein (SEQ ID NO: 101) corresponds to amino acids 115-215 of the wild-type human VEGF-C propeptide (SEQ ID NO: 1; UniProt Accession #P49767). In case of the wild-type murine VEGF-C, mature VEGF-C protein corresponds to amino acids 111-211 (SEQ ID NO: 292) of the murine VEGF-C propeptide (SEQ ID NO: 4).

In some embodiments, the VEGF-C mutein protein may be a mutein protein of a human VEGF-C protein. A human VEGF-C mutein protein may comprise a sequence that is at least or at most 70%, 80%, 85%, 86%, 87%, 88%, 89%, 90%, 91%, 92%, 93%, 94%, 95%, 96%, 97%, 98%, or 99% identical to amino acids 115-215 of the following polypeptide sequence:

MHLLGFFSVACSLLAAALLPGPREAPAAAAAFESGLDLSDAEPDAGEATAYASKDLEEQ
LRSVSSVDELMTVLYPEYWKMYKCQLRKGGWQHNREQANLNSRTEETIKFAAAHYNT
EILKSIDNEWRKTQCMPREVCIDVGKEFGVATNTFFKPPCVSVYRCGGCCNSEGLQCMN
TSTSYLSKTLFEITVPLSQGPKPVTISFANHTSCRCMSKLDVYRQVHSIIRRSLPATLPQCQ
AANKTCPTNYMWNNHICRCLAQEDFMFSSDAGDDSTDGFHDICGPNKELDEETCQCVC
RAGLRPASCGPHKELDRNSCQCVCKNKLFPSQCGANREFDENTCQCVCKRTCPRNQPL
NPGKCACECTESPQKCLLKGKKFHHQTCSCYRRPCTNRQKACEPGFSQPLNPGKCACEC
TESPQKCLLKGKKFHHQTCSCYRRPCTNRQKACEPGFS (SEQ ID NO: 1; wild-type VEGF-
C propeptide; UniProt database Accession #P49767).

The polynucleotide molecule encoding a human VEGF-C mutein protein may comprise a sequence that is at least or at most 70%, 80%, 85%, 86%, 87%, 88%, 89%, 90%, 91%, 92%, 93%, 94%, 95%, 96%, 97%, 98%, or 99% identical to nucleotides 343-645 of the following VEGF-C-encoding sequence:

ACTGGCTGGGAGGGCGCCCTGCAAAGTTGGGAACGCGGAGCCCCGGACCCGCTCCC
GCCGCCTCCGGCTCGCCCAGGGGGGGTCGCCGGGAGGAGCCCGGGGGAGAGGGAC
CAGGAGGGGCCCGCGGCCTCGCAGGGGCGCCCGCGCCCCCACCCCTGCCCCCGCCA
GCGGACCGGTCCCCCACCCCCGGTCCTTCCACCATGCACTTGCTGGGCTTCTTCTCTG
TGGCGTGTTCTCTGCTCGCCGCTGCGCTGCTCCCGGGTCCTCGCGAGGCGCCCGCCG
CCGCCGCCGCCTTCGAGTCCGGACTCGACCTCTCGGACGCGGAGCCCGACGCGGGC
GAGGCCACGGCTTATGCAAGCAAAGATCTGGAGGAGCAGTTACGGTCTGTGTCCAG
TGTAGATGAACTCATGACTGTACTCTACCCAGAATATTGGAAAATGTACAAGTGTCA
GCTAAGGAAAGGAGGCTGGCAACATAACAGAGAACAGGCCAACCTCAACTCAAGG
ACAGAAGAGACTATAAAATTTGCTGCAGCACATTATAATACAGAGATCTTGAAAAG
TATTGATAATGAGTGGAGAAAGACTCAATGCATGCCACGGGAGGTGTGTATAGATG
TGGGGAAGGAGTTTGGAGTCGCGACAAACACCTTCTTTAAACCTCCATGTGTGTCCG
TCTACAGATGTGGGGGTTGCTGCAATAGTGAGGGGCTGCAGTGCATGAACACCAGC
ACGAGCTACCTCAGCAAGACGTTATTTGAAATTACAGTGCCTCTCTCTCAAGGCCCC
AAACCAGTAACAATCAGTTTTGCCAATCACACTTCCTGCCGATGCATGTCTAAACTG
GATGTTTACAGACAAGTTCATTCCATTATTAGACGTTCCCTGCCAGCAACACTACCA
CAGTGTCAGGCAGCGAACAAGACCTGCCCCACCAATTACATGTGGAATAATCACAT
CTGCAGATGCCTGGCTCAGGAAGATTTTATGTTTTCCTCGGATGCTGGAGATGACTC
AACAGATGGATTCCATGACATCTGTGGACCAAACAAGGAGCTGGATGAAGAGACCT
GTCAGTGTGTCTGCAGAGCGGGGCTTCGGCCTGCCAGCTGTGGACCCCACAAAGAA
CTAGACAGAAACTCATGCCAGTGTGTCTGTAAAAACAAACTCTTCCCCAGCCAATGT
GGGGCCAACCGAGAATTTGATGAAAACACATGCCAGTGTGTATGTAAAAGAACCTG
CCCCAGAAATCAACCCCTAAATCCTGGAAAATGTGCCTGTGAATGTACAGAAAGTC
CACAGAAATGCTTGTTAAAAGGAAAGAAGTTCCACCACCAAACATGCAGCTGTTAC
AGACGGCCATGTACGAACCGCCAGAAGGCTTGTGAGCCAGGATTTTCATATAGTGA
AGAAGTGTGTCGTTGTGTCCCTTCATATTGGAAAAGACCACAAATGAGCTAA (SEQ
ID NO: 2; nucleotide sequence encoding human wild-type VEGF-C propeptide;
GenBank Accession No. AK313879.1).

A human VEGF-C mutein protein may comprise a sequence that is at least or at most 70%, 80%, 85%, 86%, 87%, 88%, 89%, 90%, 91%, 92%, 93%, 94%, 95%, 96%, 97%, 98%, or 99% identical to the following polypeptide sequence:

NTEILKSIDNEWRKTQCMPREVCIDVGKEFGVATNTFFKPPCVSVYRCGGCCNSEGLQC
MNTSTSYLSKTLFEITVPLSQGPKPVTISFANHTSCRCMSKL (wild-type human mature
VEGF-C protein; SEQ ID NO: 101).

The polynucleotide molecule encoding a human VEGF-C mutein protein may comprise a sequence that is at least or at most 70%, 80%, 85%, 86%, 87%, 88%, 89%, 90%, 91%, 92%, 93%, 94%, 95%, 96%, 97%, 98%, or 99% identical to the following VEGF-C-encoding sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCAATTCTGAGGGGCT
CCAATGTATGAACACGAGTACGTCTTACTTGAGTAAGACCTTGTTCGAAATTACAGT
ACCACTTTCCCAAGGACCGAAGCCTGTGACTATCAGCTTTGCTAACCATACAAGCTG
TCGCTGTATGAGCAAACTG (SEQ ID NO: 153; nucleotide sequence encoding human
mature VEGF-C protein).

In certain embodiments, the VEGF-C mutein comprises one or more mutations at residues T116, L119, D123, Q130, T148, N149, K153, N167, S168, E169, I188, V190, L192, P196 and combinations thereof, wherein the positions of mutations are defined in relation to SEQ ID NO: 1. In certain embodiments, the mutation at L119 is a L119E mutation. In certain embodiments, the mutation at L119 is a L119M mutation. In certain embodiments, the mutation at D123 is a D123N mutation. In certain embodiments, the mutation at Q130 is a Q130K mutation. In certain embodiments, the mutation at N167 is a N167R mutation. In certain embodiments, the mutation at N167 is a N167I mutation. In certain embodiments, the mutation at N167 is a N167Q mutation. In certain embodiments, the mutation at N167 is a N167H mutation. In certain embodiments, the mutation at S168 is a S168G mutation. In certain embodiments, the mutation at S168 is a S168R mutation. In certain embodiments, the mutation at V190 is a V190T mutation. In certain embodiments, the mutation at L192L is a L192I mutation.

In some embodiments, the VEGF-C mutein protein may be a mutein protein of a murine VEGF-C protein. In certain embodiments, a murine VEGF-C mutein may comprise a sequence that is at least or at most 70%, 80%, 85%, 86%, 87%, 88%, 89%, 90%, 91%, 92%, 93%, 94%, 95%, 96%, 97%, 98%, or 99% identical to amino acids 111-211 of the following polypeptide sequence:

MHLLCFLSLACSLLAAALIPSPREAPATVAAFESGLGFSEAEPDGGEVKAFEGKDLEEQL
RSVSSVDELMSVLYPDYWKMYKCQLRKGGWQQPTLNTRTGDSVKFAAAHYNTEILKSI
DNEWRKTQCMPREVCIDVGKEFGAATNTFFKPPCVSVYRCGGCCNSEGLQCMNTSTGY
LSKTLFEITVPLSQGPKPVTISFANHTSCRCMSKLDVYRQVHSIIRRSLPATLPQCQAANK
TCPTNYVWNNYMCRCLAQQDFIFYSNVEDDSTNGFHDVCGPNKELDEDTCQCVCKGG
LRPSSCGPHKELDRDSCQCVCKNKLFPNSCGANREFDENTCQCVCKRTCPRNQPLNPGK
CACECTENTQKCFLKGKKFHHQTCSCYRRPCANRLKHCDPGLSFSEEVCRCVPSYWKR
PHLN (murine wild type VEGF-C propeptide; SEQ ID NO: 4).

The polynucleotide molecule encoding a murine VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% identical to nucleotides 331-631 of the following VEGF-C-encoding sequence:

ATGCACTTGCTGTGCTTCTTGTCTCTGGCGTGTTCCCTGCTCGCCGCTGCGCTGATCC
CCAGTCCGCGCGAGGCGCCCGCCACCGTCGCCGCCTTCGAGTCGGGACTGGGCTTCT
CGGAAGCGGAGCCCGACGGGGGCGAGGTCAAGGCTTTTGAAGGCAAAGACCTGGA
GGAGCAGTTGCGGTCTGTGTCCAGCGTAGATGAGCTGATGTCTGTCCTGTACCCAGA
CTACTGGAAAATGTACAAGTGCCAGCTGCGGAAAGGCGGCTGGCAGCAGCCCACCC
TCAATACCAGGACAGGGGACAGTGTAAAATTTGCTGCTGCACATTATAACACAGAG
ATCCTGAAAAGTATTGATAATGAGTGGAGAAAGACTCAATGCATGCCACGTGAGGT
GTGTATAGATGTGGGGAAGGAGTTTGGAGCAGCCACAAACACCTTCTTTAAACCTCC
ATGTGTGTCCGTCTACAGATGTGGGGGTTGCTGCAACAGCGAGGGGCTGCAGTGCAT
GAACACCAGCACAGGTTACCTCAGCAAGACGTTGTTTGAAATTACAGTGCCTCTCTC
ACAAGGCCCCAAACCAGTCACAATCAGTTTTGCCAATCACACTTCCTGCCGGTGCAT
GTCTAAACTGGATGTTTACAGACAAGTTCATTCAATTATTAGACGTTCTCTGCCAGC
AACATTACCACAGTGTCAGGCAGCTAACAAGACATGTCCAACAAACTATGTGTGGA
ATAACTACATGTGCCGATGCCTGGCTCAGCAGGATTTTATCTTTTATTCAAATGTTGA
AGATGACTCAACCAATGGATTCCATGATGTCTGTGGACCCAACAAGGAGCTGGATG
AAGACACCTGTCAGTGTGTCTGCAAGGGGGGGCTTCGGCCATCTAGTTGTGGACCCC
ACAAAGAACTAGATAGAGACTCATGTCAGTGTGTCTGTAAAAACAAACTTTTCCCTA
ATTCATGTGGAGCCAACAGGGAATTTGATGAGAATACATGTCAGTGTGTATGTAAA
AGAACGTGTCCAAGAAATCAGCCCCTGAATCCTGGGAAATGTGCCTGTGAATGTAC
AGAAAACACACAGAAGTGCTTCCTTAAAGGGAAGAAGTTCCACCATCAAACATGCA
GTTGTTACAGAAGACCGTGTGCGAATCGACTGAAGCATTGTGATCCAGGACTGTCCT
TTAGTGAAGAAGTATGCCGCTGTGTCCCATCGTATTGGAAAAGGCCACATCTGAACT
AA (nucleotide sequence encoding murine wild type VEGF-C propeptide; SEQ
ID NO: 3).

In some embodiments, the VEGF-C mutein protein may be a mutein protein of a murine VEGF-C protein. In certain embodiments, a murine VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% identical to the following polypeptide sequence:

NTEILKSIDNEWRKTQCMPREVCIDVGKEFGAATNTFFKPPCVSVYRCGGCCNSEGLQC
MNTSTGYLSKTLFEITVPLSQGPKPVTISFANHTSCRCMSKL (wild-type murine mature
VEGF-C protein; SEQ ID NO: 292).

The polynucleotide molecule encoding a murine VEGF-C mutein protein may comprise a sequence that is at least or at most 70%, 80%, 85%, 86%, 87%, 88%, 89%, 90%, 91%, 92%, 93%, 94%, 95%, 96%, 97%, 98%, 99% identical to the following VEGF-C-encoding sequence:

AACACAGAGATCCTGAAAAGTATTGATAATGAGTGGAGAAAGACTCAATGCATGCC
ACGTGAGGTGTGTATAGATGTGGGGAAGGAGTTTGGAGCAGCCACAAACACCTTCT
TTAAACCTCCATGTGTGTCCGTCTACAGATGTGGGGGTTGCTGCAACAGCGAGGGGC
TGCAGTGCATGAACACCAGCACAGGTTACCTCAGCAAGACGTTGTTTGAAATTACAG
TGCCTCTCTCACAAGGCCCCAAACCAGTCACAATCAGTTTTGCCAATCACACTTCCT
GCCGGTGCATGTCTAAACTG (nucleotide sequence encoding wild-type murine
mature VEGF-C protein; SEQ ID NO: 293).

In certain embodiments, the VEGF-C mutein protein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCQSEGLQC
MNTSTSYLSKTLFEITVPLSQGPKPVTISFANHTSCRCMSKL (human N167Q VEGF-C
mutein protein; SEQ ID NO: 160).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCCAGTCTGAGGGGCT
CCAATGTATGAACACGAGTACGtctTACTTGAGTAAGACCTTGTTCGAAATTACAGTA
CCACTTTCCCAAGGACCGAAGCCTGTGACTATCAGCTTTGCTAACCATACAAGCTGT
CGCTGTATGAGCAAACTG (nucleotide sequence encoding human N167Q VEGF-C
mutein protein; SEQ ID NO: 210).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCQGEGLQC
MNTSTSYLSKTLFEITVPLSQGPKPVTISFANHTSCRCMSKL (human N167Q and S168G
VEGF-C mutein protein; SEQ ID NO: 161).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCCAGGGCGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding human N167Q and
S168G VEGF-C mutein protein; SEQ ID NO: 211).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCQSEGLQC
MNTSTSYLSKTLFEITVPISQGPKPVTISFANHTSCRCMSKL (human N167Q and L192I
VEGF-C mutein protein; SEQ ID NO: 162).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCCAGTCTGAGGGGCT
CCAATGTATGAACACGAGTACGTCTTACTTGAGTAAGACCTTGTTCGAAATTACAGT
ACCAATCTCCCAAGGACCGAAGCCTGTGACTATCAGCTTTGCTAACCATACAAGCTG
TCGCTGTATGAGCAAACTG (nucleotide sequence encoding human N167Q and L192I
VEGF-C mutein protein; SEQ ID NO: 212).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCQGEGLQC
MNTSTSYLSKTLFEITVPISQGPKPVTISFANHTSCRCMSKL (human N167Q, S168G, and
L192I VEGF-C mutein protein; SEQ ID NO: 163).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCCAGGGCGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTG (nucleotide sequence encoding human N167Q, S168G,
and L192I VEGF-C mutein protein; SEQ ID NO: 213).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCISEGLQC
MNTSTSYLSKTLFEITVPLSQGPKPVTISFANHTSCRCMSKL (human N167I VEGF-C
mutein protein; SEQ ID NO: 166).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCATCTCTGAGGGGCT
CCAATGTATGAACACGAGTACGTCTTACTTGAGTAAGACCTTGTTCGAAATTACAGT
ACCACTTTCCCAAGGACCGAAGCCTGTGACTATCAGCTTTGCTAACCATACAAGCTG
TCGCTGTATGAGCAAACTG (nucleotide sequence encoding human N167I VEGF-C
mutein protein; SEQ ID NO: 216).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCIGEGLQC
MNTSTSYLSKTLFEITVPLSQGPKPVTISFANHTSCRCMSKL (human N1671 and S168G
VEGF-C mutein protein; SEQ ID NO: 167).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCATCGGCGAGGGGCT
CCAATGTATGAACACGAGTACGTCTTACTTGAGTAAGACCTTGTTCGAAATTACAGT
ACCACTTTCCCAAGGACCGAAGCCTGTGACTATCAGCTTTGCTAACCATACAAGCTG
TCGCTGTATGAGCAAACTG (nucleotide sequence encoding human N167I and S168G
VEGF-C mutein protein; SEQ ID NO: 217).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCISEGLQC
MNTSTSYLSKTLFEITVPISQGPKPVTISFANHTSCRCMSKL (human N1671 and L192I
VEGF-C mutein protein; SEQ ID NO: 168).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCATCTCTGAGGGGCT
CCAATGTATGAACACGAGTACGTCTTACTTGAGTAAGACCTTGTTCGAAATTACAGT
ACCAATCTCCCAAGGACCGAAGCCTGTGACTATCAGCTTTGCTAACCATACAAGCTG
TCGCTGTATGAGCAAACTG (nucleotide sequence encoding human N1671 and L192I
VEGF-C mutein protein; SEQ ID NO: 218).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCIGEGLQC
MNTSTSYLSKTLFEITVPISQGPKPVTISFANHTSCRCMSKL (human N167I, S168G, and
L192I VEGF-C mutein protein; SEQ ID NO: 169).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCATCGGCGAGGGGCT
CCAATGTATGAACACGAGTACGTCTTACTTGAGTAAGACCTTGTTCGAAATTACAGT
ACCAATCTCCCAAGGACCGAAGCCTGTGACTATCAGCTTTGCTAACCATACAAGCTG
TCGCTGTATGAGCAAACTG (nucleotide sequence encoding human N167I, S168G and
L192I VEGF-C mutein protein; SEQ ID NO: 219).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCNGEGLQC
MNTSTSYLSKTLFEITVPLSQGPKPVTISFANHTSCRCMSKL (human S168G VEGF-C
mutein protein; SEQ ID NO: 172).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCAATGGCGAGGGGCT
CCAATGTATGAACACGAGTACGTCTTACTTGAGTAAGACCTTGTTCGAAATTACAGT
ACCACTTTCCCAAGGACCGAAGCCTGTGACTATCAGCTTTGCTAACCATACAAGCTG
TCGCTGTATGAGCAAACTG (nucleotide sequence encoding human S168G VEGF-C
mutein protein; SEQ ID NO: 222).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCNGEGLQC
MNTSTSYLSKTLFEITVPISQGPKPVTISFANHTSCRCMSKL (human S168G and L192I
VEGF-C mutein protein; SEQ ID NO: 174).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCAATGGCGAGGGGCT
CCAATGTATGAACACGAGTACGTCTTACTTGAGTAAGACCTTGTTCGAAATTACAGT
ACCAATCTCCCAAGGACCGAAGCCTGTGACTATCAGCTTTGCTAACCATACAAGCTG
TCGCTGTATGAGCAAACTG (nucleotide sequence encoding human S168G and
L192I VEGF-C mutein protein; SEQ ID NO: 224).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCHSEGLQC
MNTSTSYLSKTLFEITVPLSQGPKPVTISFANHTSCRCMSKL (human N167H VEGF-C
mutein protein; SEQ ID NO: 184).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCCACTCTGAGGGGCT
CCAATGTATGAACACGAGTACGTCTTACTTGAGTAAGACCTTGTTCGAAATTACAGT
ACCACTTTCCCAAGGACCGAAGCCTGTGACTATCAGCTTTGCTAACCATACAAGCTG
TCGCTGTATGAGCAAACTG (nucleotide sequence encoding human N167H VEGF-C
mutein protein; SEQ ID NO: 234).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGVATNTFFKPPCVSVYRCGGCCIREGLQC
MNTSTSYLSKTLFEITVPLSQGPKPVTISFANHTSCRCMSKL (human N167I and S168R
VEGF-C mutein protein; SEQ ID NO: 192).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGTAGCAACGAACACATTCTT
CAAACCCCCTTGTGTGAGTGTATATAGATGTGGAGGGTGTTGCATCAGAGAGGGGCT
CCAATGTATGAACACGAGTACGTCTTACTTGAGTAAGACCTTGTTCGAAATTACAGT
ACCACTTTCCCAAGGACCGAAGCCTGTGACTATCAGCTTTGCTAACCATACAAGCTG
TCGCTGTATGAGCAAACTG (nucleotide sequence encoding human N167I and
S168R VEGF-C mutein protein; SEQ ID NO: 242).

In some embodiments, the VEGF-C mutein protein or functional fragment thereof comprises one or more mutations selected from mutations at residues N167, S168, and/or L192, wherein the positions of mutations are defined in relation to SEQ ID NO: 1.

In some embodiments, the mutation at N167 is N167I mutation, N167Q mutation, or N167H mutation; the mutation at S168 is S168G mutation, or S168R mutation; and/or the mutation at L192 is L192I mutation, wherein the positions of mutations are defined in relation to SEQ ID NO: 1.

In some embodiments, the VEGF-C mutein protein or functional fragment thereof comprises N167Q mutation, wherein the position of the mutation is defined in relation to SEQ ID NO: 1.

In some embodiments, the VEGF-C mutein protein or functional fragment thereof comprises N167Q mutation and S168G mutation, wherein the positions of mutations are defined in relation to SEQ ID NO: 1.

In some embodiments, the VEGF-C mutein protein or functional fragment thereof comprises N167Q mutation and L192I mutation, wherein the positions of mutations are defined in relation to SEQ ID NO: 1.

In some embodiments, the VEGF-C mutein protein or functional fragment thereof comprises N167Q mutation, S168G mutation, and L192I mutation, wherein the positions of mutations are defined in relation to SEQ ID NO: 1.

In some embodiments, the VEGF-C mutein protein or functional fragment thereof comprises N167I mutation, wherein the position of the mutation is defined in relation to SEQ ID NO: 1.

In some embodiments, the VEGF-C mutein protein or functional fragment thereof comprises N167I mutation and S168G mutation, wherein the positions of mutations are defined in relation to SEQ ID NO: 1.

In some embodiments, the VEGF-C mutein protein or functional fragment thereof comprises N167I mutation and L192I mutation, wherein the positions of mutations are defined in relation to SEQ ID NO: 1.

In some embodiments, the VEGF-C mutein protein or functional fragment thereof comprises N167I mutation, S168G mutation, and L192I mutation, wherein the positions of mutations are defined in relation to SEQ ID NO: 1.

In some embodiments, the VEGF-C mutein protein or functional fragment thereof comprises S168G mutation, wherein the position of the mutation is defined in relation to SEQ ID NO: 1.

In some embodiments, the VEGF-C mutein protein or functional fragment thereof comprises S168G mutation and L192I mutation, wherein the positions of mutations are defined in relation to SEQ ID NO: 1.

In some embodiments, the VEGF-C mutein protein or functional fragment thereof comprises N167H mutation, wherein the positions of mutations are defined in relation to SEQ ID NO: 1.

In some embodiments, the VEGF-C mutein protein or functional fragment thereof comprises N167I mutation and S168R mutation, wherein the positions of mutations are defined in relation to SEQ ID NO: 1.

In certain embodiments, the VEGF-C mutein may comprise a functional fragment of the human VEGF-C.

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the polypeptide sequence of any of the SEQ ID NOs: 154-159, SEQ ID NOs: 164-165, SEQ ID NOs: 170-171, SEQ ID NO: 173, SEQ ID NOs: 175-183, SEQ ID NOs: 185-191, or SEQ ID NOs: 193-203.

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the any of SEQ ID NOs: 204-209, SEQ ID NOs: 214-215, SEQ ID NOs: 220-221, SEQ ID NO: 223, SEQ ID NOs: 225-233, SEQ ID NOS: 235-241, or SEQ ID NOs: 243-253.

In certain embodiments, the VEGF-C mutein may comprise a functional fragment of the murine VEGF-C. In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVCIDVGKEFGAATNTFFKPPCVSVYRCGGCCNSEGLQC
MNTSTGYLSKTLFEITVPLSQGPKPVTISFANHTSCRCMSKL (murine VEGF-C mutein
protein, SEQ ID NO: 23).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTATGTATCGACGTTGGTAAAGAATTTGGTGCGGCAACGAACACATTCTT
CAAACCCCCTTGTGTGAGTGTATATAGATGTGGAGGGTGTTGCAATTCTGAGGGGCT
CCAATGTATGAACACGAGTACGGGTTACTTGAGTAAGACCTTGTTCGAAATTACAGT
ACCACTTTCCCAAGGACCGAAGCCTGTGACTATCAGCTTTGCTAACCATACAAGCTG
TCGCTGTATGAGCAAACTG (nucleotide sequence encoding murine
VEGF-C mutein protein, SEQ ID NO: 24).

In certain embodiments, the VEGF-C mutein comprises one or more mutations at residues T112, L115, D119, Q126, T144, N145, K149, N163, S164, E165, I184, V186, L188, P192 and combinations thereof, wherein the positions of mutations are defined in relation to SEQ ID NO: 4. In certain embodiments, the mutation at L115 is a L115E mutation. In certain embodiments, the mutation at L115 is a L115M mutation. In certain embodiments, the mutation at D119 is a D119N mutation. In certain embodiments, the mutation at Q126 is a Q126K mutation. In certain embodiments, the mutation at N163 is a N163R mutation. In certain embodiments, the mutation at N163 is a N163I mutation. In certain embodiments, the mutation at N163 is a N163Q mutation. In certain embodiments, the mutation at S164 is a S164G mutation. In certain embodiments, the mutation at V186 is a V184T mutation. In certain embodiments, the mutation at L188 is a L188I mutation. In some embodiments, the VEGF-C mutein protein comprises mutations N163I.

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

MHLLCFLSLACSLLAAALIPSPREAPATVAAFESGLGFSEAEPDGGEVKAFEGKDLEEQL
RSVSSVDELMSVLYPDYWKMYKCQLRKGGWQQPTLNTRTGDSVKFAAAHYNTEILKSI
DNEWRKTQCMPREVCIDVGKEFGAATNTFFKPPCVSVYRCGGCCISEGLQCMNTSTGY
LSKTLFEITVPLSQGPKPVTISFANHTSCRCMSKLDVYRQVHSIIRRSLPATLPQCQAANK
TCPTNYVWNNYMCRCLAQQDFIFYSNVEDDSTNGFHDVCGPNKELDEDTCQCVCKGG
LRPSSCGPHKELDRDSCQCVCKNKLFPNSCGANREFDENTCQCVCKRTCPRNQPLNPGK
CACECTENTQKCFLKGKKFHHQTCSCYRRPCANRLKHCDPGLSFSEEVCRCVPSYWKR
PHLN (murine N163I VEGF-C mutein protein; SEQ ID NO: 6).

In certain embodiments, the polynucleotide molecule encoding the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following nucleotide sequence:

ATGCACTTGCTGTGCTTCTTGTCTCTGGCGTGTTCCCTGCTCGCCGCTGCGCTGATCC
CCAGTCCGCGCGAGGCGCCCGCCACCGTCGCCGCCTTCGAGTCGGGACTGGGCTTCT
CGGAAGCGGAGCCCGACGGGGGCGAGGTCAAGGCTTTTGAAGGCAAAGACCTGGA
GGAGCAGTTGCGGTCTGTGTCCAGCGTAGATGAGCTGATGTCTGTCCTGTACCCAGA
CTACTGGAAAATGTACAAGTGCCAGCTGCGGAAAGGCGGCTGGCAGCAGCCCACCC
TCAATACCAGGACAGGGGACAGTGTAAAATTTGCTGCTGCACATTATAACACAGAG
ATCCTGAAAAGTATTGATAATGAGTGGAGAAAGACTCAATGCATGCCACGTGAGGT
GTGTATAGATGTGGGGAAGGAGTTTGGAGCAGCCACAAACACCTTCTTTAAACCTCC
ATGTGTGTCCGTCTACAGATGTGGGGGTTGCTGCATCAGCGAGGGGCTGCAGTGCAT
GAACACCAGCACAGGTTACCTCAGCAAGACGTTGTTTGAAATTACAGTGCCTCTCTC
ACAAGGCCCCAAACCAGTCACAATCAGTTTTGCCAATCACACTTCCTGCCGGTGCAT
GTCTAAACTGGATGTTTACAGACAAGTTCATTCAATTATTAGACGTTCTCTGCCAGC
AACATTACCACAGTGTCAGGCAGCTAACAAGACATGTCCAACAAACTATGTGTGGA
ATAACTACATGTGCCGATGCCTGGCTCAGCAGGATTTTATCTTTTATTCAAATGTTGA
AGATGACTCAACCAATGGATTCCATGATGTCTGTGGACCCAACAAGGAGCTGGATG
AAGACACCTGTCAGTGTGTCTGCAAGGGGGGGCTTCGGCCATCTAGTTGTGGACCCC
ACAAAGAACTAGATAGAGACTCATGTCAGTGTGTCTGTAAAAACAAACTTTTCCCTA
ATTCATGTGGAGCCAACAGGGAATTTGATGAGAATACATGTCAGTGTGTATGTAAA
AGAACGTGTCCAAGAAATCAGCCCCTGAATCCTGGGAAATGTGCCTGTGAATGTAC
AGAAAACACACAGAAGTGCTTCCTTAAAGGGAAGAAGTTCCACCATCAAACATGCA
GTTGTTACAGAAGACCGTGTGCGAATCGACTGAAGCATTGTGATCCAGGACTGTCCT
TTAGTGAAGAAGTATGCCGCTGTGTCCCATCGTATTGGAAAAGGCCACATCTGAACT
AA (nucleotide sequence encoding murine N1631 VEGF-C mutein protein,
SEQ ID NO: 5).

In some embodiments, the VEGF-C mutein protein comprises mutations N163R, V186T and L188I, wherein the positions of mutations are defined in relation to SEQ ID NO: 4.

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

MHLLCFLSLACSLLAAALIPSPREAPATVAAFESGLGFSEAEPDGGEVKAFEGKDLEEQL
RSVSSVDELMSVLYPDYWKMYKCQLRKGGWQQPTLNTRTGDSVKFAAAHYNTEILKSI
DNEWRKTQCMPREVCIDVGKEFGAATNTFFKPPCVSVYRCGGCCRSEGLQCMNTSTGY
LSKTLFEITTPISQGPKPVTISFANHTSCRCMSKLDVYRQVHSIIRRSLPATLPQCQAANKT
CPTNYVWNNYMCRCLAQQDFIFYSNVEDDSTNGFHDVCGPNKELDEDTCQCVCKGGL
RPSSCGPHKELDRDSCQCVCKNKLFPNSCGANREFDENTCQCVCKRTCPRNQPLNPGKC
ACECTENTQKCFLKGKKFHHQTCSCYRRPCANRLKHCDPGLSFSEEVCRCVPSYWKRP
HLN (murine RTI VEGF-C mutein protein, SEQ ID NO: 8).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99% or 100% sequence identity to the following nucleotide sequence:

ATGCACTTGCTGTGCTTCTTGTCTCTGGCGTGTTCCCTGCTCGCCGCTGCGCTGATCC
CCAGTCCGCGCGAGGCGCCCGCCACCGTCGCCGCCTTCGAGTCGGGACTGGGCTTCT
CGGAAGCGGAGCCCGACGGGGGCGAGGTCAAGGCTTTTGAAGGCAAAGACCTGGA
GGAGCAGTTGCGGTCTGTGTCCAGCGTAGATGAGCTGATGTCTGTCCTGTACCCAGA
CTACTGGAAAATGTACAAGTGCCAGCTGCGGAAAGGCGGCTGGCAGCAGCCCACCC
TCAATACCAGGACAGGGGACAGTGTAAAATTTGCTGCTGCACATTATAACACAGAG
ATCCTGAAAAGTATTGATAATGAGTGGAGAAAGACTCAATGCATGCCACGTGAGGT
GTGTATAGATGTGGGGAAGGAGTTTGGAGCAGCCACAAACACCTTCTTTAAACCTCC
ATGTGTGTCCGTCTACAGATGTGGGGGTTGCTGCAGAAGCGAGGGGCTGCAGTGCA
TGAACACCAGCACAGGTTACCTCAGCAAGACGTTGTTTGAAATTACAACTCCTATTT
CACAAGGCCCCAAACCAGTCACAATCAGTTTTGCCAATCACACTTCCTGCCGGTGCA
TGTCTAAACTGGATGTTTACAGACAAGTTCATTCAATTATTAGACGTTCTCTGCCAGC
AACATTACCACAGTGTCAGGCAGCTAACAAGACATGTCCAACAAACTATGTGTGGA
ATAACTACATGTGCCGATGCCTGGCTCAGCAGGATTTTATCTTTTATTCAAATGTTGA
AGATGACTCAACCAATGGATTCCATGATGTCTGTGGACCCAACAAGGAGCTGGATG
AAGACACCTGTCAGTGTGTCTGCAAGGGGGGGCTTCGGCCATCTAGTTGTGGACCCC
ACAAAGAACTAGATAGAGACTCATGTCAGTGTGTCTGTAAAAACAAACTTTTCCCTA
ATTCATGTGGAGCCAACAGGGAATTTGATGAGAATACATGTCAGTGTGTATGTAAA
AGAACGTGTCCAAGAAATCAGCCCCTGAATCCTGGGAAATGTGCCTGTGAATGTAC
AGAAAACACACAGAAGTGCTTCCTTAAAGGGAAGAAGTTCCACCATCAAACATGCA
GTTGTTACAGAAGACCGTGTGCGAATCGACTGAAGCATTGTGATCCAGGACTGTCCT
TTAGTGAAGAAGTATGCCGCTGTGTCCCATCGTATTGGAAAAGGCCACATCTGAACT
AA (nucleotide sequence encoding murine RTI VEGF-C mutein protein,
SEQ ID NO: 7).

In certain embodiments, the VEGF-C mutein comprises a mutation at residue C133, wherein the position of the mutation is defined in relation to SEQ ID NO: 4. In certain embodiments, the mutation at residue C133 is a C133A mutation.

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

NTEILKSIDNEWRKTQCMPREVAIDVGKEFGAATNTFFKPPCVSVYRCGGCCNSEGLQC
MNTSTGYLSKTLFEITVPLSQGPKPVTISFANHTSCRCMSKL (murine VEGF-C mutein
protein with C133A mutation, SEQ ID NO: 9).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGCGGCAACGAACACATTCT
TCAAACCCCCTTGTGTGAGTGTATATAGATGTGGAGGGTGTTGCAATTCTGAGGGGC
TCCAATGTATGAACACGAGTACGGGTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding murine
VEGF-C mutein protein with C133A mutation, SEQ ID NO: 10).

In certain embodiments, the VEGF-C mutein protein comprises mutations C133A and N163I, wherein the positions of mutations are defined in relation to SEQ ID NO: 4.

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

NTEILKSIDNEWRKTQCMPREVAIDVGKEFGAATNTFFKPPCVSVYRCGGCCISEGLQC
MNTSTGYLSKTLFEITVPLSQGPKPVTISFANHTSCRCMSKL (murine VEGF-C mutein
protein with C133A and N163I mutations, SEQ ID NO: 17).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99% or 100% sequence identity to the following

AACACCGAAATCTTGAAATCAATAGATAACGAATGGCGCAAAACTCAATGTATGCC
ACGGGAGGTTGCAATAGACGTGGGAAAGGAATTTGGCGCCGCCACGAATACCTTTT
TCAAGCCTCCCTGTGTGAGTGTTTACAGATGTGGTGGTTGTTGCATATCAGAGGGAT
TGCAGTGCATGAACACAAGTACAGGTTACTTGAGTAAAACATTGTTTGAAATCACAG
TACCATTGTCCCAAGGCCCTAAGCCGGTTACGATCTCTTTCGCCAATCATACGTCAT
GCCGCTGTATGAGTAAGTTG (nucleotide sequence encoding murine VEGF-C
mutein protein with C133A and N163I mutations, SEQ ID NO: 18).

In some embodiments, the VEGF-C mutein protein comprises mutations C133A, N163R, V186T and L188I, wherein the positions of mutations are defined in relation to SEQ ID NO: 4.

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

NTEILKSIDNEWRKTQCMPREVAIDVGKEFGAATNTFFKPPCVSVYRCGGCCRSEGLQC
MNTSTGYLSKTLFEITTPISQGPKPVTISFANHTSCRCMSKL (murine VEGF-C mutein
protein with C133A, N163R, V186T, and L188I mutations, SEQ ID NO: 13).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99%, or 100% sequence identity to the following nucleotide sequence:

AACACCGAAATCTTGAAATCAATAGATAACGAATGGCGAAAAACCCAATGTATGCC
ACGCGAGGTAGCGATAGATGTGGGCAAAGAATTCGGCGCCGCGACGAACACCTTTT
TCAAGCCCCCTTGCGTCTCCGTATATAGATGCGGTGGATGTTGCCGATCCGAGGGCC
TTCAGTGTATGAACACATCTACTGGCTATTTGAGCAAGACGCTCTTTGAGATTACAA
CACCAATTAGTCAAGGTCCCAAGCCTGTTACCATCTCTTTCGCTAACCACACTTCAT
GCCGCTGTATGAGTAAGTTG (nucleotide sequence encoding murine VEGF-C mutein
protein with C133A, N163R, V186T, and L188I mutations, SEQ ID NO: 14).

In certain embodiments, the VEGF-C mutein comprises a mutation at residue C137, wherein the position of the mutation is defined in relation to SEQ ID NO: 1. In certain embodiments, the mutation at residue C137 is a C1337A mutation.

In certain embodiments, the human VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQSEGLQC
MNTSTSYLSKTLFEITVPLSQGPKPVTISFANHTSCRCMSKL (human VEGF-C mutein
protein with C133A mutation; SEQ ID NO: 100).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCAATTCTGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding human VEGF-C
mutein protein with C133A mutation, SEQ ID NO: 152).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQSEGLQC
MNTSTSYLSKTLFEITVPLSQGPKPVTISFANHTSCRCMSKL (human C137A and N167Q
VEGF-C mutein protein; SEQ ID NO: 56).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCAGTCTGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding human C137A and
N167Q VEGF-C mutein protein; SEQ ID NO: 108).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQGEGLQC
MNTSTSYLSKTLFEITVPLSQGPKPVTISFANHTSCRCMSKL (human C137A, N167Q and
S168G VEGF-C mutein protein; SEQ ID NO: 57).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCAGGGCGAGGGG
CTCCAATGTATGAACACGAGTACGTCTTACTTGAGTAAGACCTTGTTCGAAATTACA
GTACCACTTTCCCAAGGACCGAAGCCTGTGACTATCAGCTTTGCTAACCATACAAGC
TGTCGCTGTATGAGCAAACTG (nucleotide sequence encoding human C137A,
N167Q and S168G VEGF-C mutein protein; SEQ ID NO: 109).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQSEGLQC
MNTSTSYLSKTLFEITVPISQGPKPVTISFANHTSCRCMSKL (human C137A, N167Q and
L192I VEGF-C mutein protein; SEQ ID NO: 58).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCAGTCTGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTG (nucleotide sequence encoding human C137A, N167Q
and L192I VEGF-C mutein protein; SEQ ID NO: 110).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQGEGLQC
MNTSTSYLSKTLFEITVPISQGPKPVTISFANHTSCRCMSKL (human C137A, N167Q,
S168G and L192I VEGF-C mutein protein; SEQ ID NO: 59).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCAGGGCGAGGGG
CTCCAATGTATGAACACGAGTACGTCTTACTTGAGTAAGACCTTGTTCGAAATTACA
GTACCAATCTCCCAAGGACCGAAGCCTGTGACTATCAGCTTTGCTAACCATACAAGC
TGTCGCTGTATGAGCAAACTG (nucleotide sequence encoding human C137A,
N167Q, S168G and L192I VEGF-C mutein protein; SEQ ID NO: 111).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCISEGLQC
MNTSTSYLSKTLFEITVPLSQGPKPVTISFANHTSCRCMSKL (human C137A and N167I
VEGF-C mutein protein; SEQ ID NO: 62).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCTCTGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding human C137A and
N167I VEGF-C mutein protein; SEQ ID NO: 114).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCIGEGLQC
MNTSTSYLSKTLFEITVPLSQGPKPVTISFANHTSCRCMSKL (human C137A, N167I and
S168G VEGF-C mutein protein; SEQ ID NO: 63).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCGGCGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding human C137A,
N167I and S168G VEGF-C mutein protein; SEQ ID NO: 115).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCISEGLQC
MNTSTSYLSKTLFEITVPISQGPKPVTISFANHTSCRCMSKL (human C137A, N167I and
L192I VEGF-C mutein protein; SEQ ID NO: 64).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCTCTGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTG (nucleotide sequence encoding human C137A,
N167I and L192I VEGF-C mutein protein; SEQ ID NO: 116).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCIGEGLQC
MNTSTSYLSKTLFEITVPISQGPKPVTISFANHTSCRCMSKL (human C137A, N167I,
S168G and L192I VEGF-C mutein protein; SEQ ID NO: 65).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCGGCGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTG (nucleotide sequence encoding human C137A, N167I,
S168G and L192I VEGF-C mutein protein; SEQ ID NO: 117).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCNGEGLQC
MNTSTSYLSKTLFEITVPLSQGPKPVTISFANHTSCRCMSKL (human C137A and S168G
VEGF-C mutein protein; SEQ ID NO: 68).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCAATGGCGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding human C137A and
S168G VEGF-C mutein protein; SEQ ID NO: 120).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCNGEGLQC
MNTSTSYLSKTLFEITVPISQGPKPVTISFANHTSCRCMSKL (human C137A, S168G and
L192I VEGF-C mutein protein; SEQ ID NO: 70).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCAATGGCGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTG (nucleotide sequence encoding human C137A,
S168G and L192I VEGF-C mutein protein; SEQ ID NO: 122).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCHSEGLQC
MNTSTSYLSKTLFEITVPLSQGPKPVTISFANHTSCRCMSKL (human C137A and N167H
VEGF-C mutein protein; SEQ ID NO: 80).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCACTCTGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding human C137A and
N167H VEGF-C mutein protein; SEQ ID NO: 132).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCIREGLQC
MNTSTSYLSKTLFEITVPLSQGPKPVTISFANHTSCRCMSKL (human C137A, N167I and
S168R VEGF-C mutein protein; SEQ ID NO: 88).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCAGAGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTG (nucleotide sequence encoding human C137A,
N167I and S168R VEGF-C mutein protein; SEQ ID NO: 140).

In some embodiments, the VEGF-C mutein protein or functional fragment thereof comprises one or more mutations selected from mutations at residues C137, N167, S168, and/or L192, wherein the positions of mutations are defined in relation to SEQ ID NO: 1.

In some embodiments, the mutation at C137 is C137A, the mutation at N167 is N167I mutation, N167Q mutation, or N167H mutation; the mutation at S168 is S168G mutation, or S168R mutation; and/or the mutation at L192 is L192I mutation, wherein the positions of mutations are defined in relation to SEQ ID NO: 1.

In some embodiments, the VEGF-C mutein protein or functional fragment thereof comprises C137A mutation and N167Q mutation, wherein the positions of mutations are defined in relation to SEQ ID NO: 1.

In some embodiments, the VEGF-C mutein protein or functional fragment thereof comprises C137A mutation, N167Q mutation and S168G mutation, wherein the positions of mutations are defined in relation to SEQ ID NO: 1.

In some embodiments, the VEGF-C mutein protein or functional fragment thereof comprises C137A mutation, N167Q mutation and L192I mutation, wherein the positions of mutations are defined in relation to SEQ ID NO: 1.

In some embodiments, the VEGF-C mutein protein or functional fragment thereof comprises C137A mutation, N167Q mutation, S168G mutation, and L192I mutation, wherein the positions of mutations are defined in relation to SEQ ID NO: 1.

In some embodiments, the VEGF-C mutein protein or functional fragment thereof comprises C137A mutation and N167I mutation, wherein the positions of mutations are defined in relation to SEQ ID NO: 1.

In some embodiments, the VEGF-C mutein protein or functional fragment thereof comprises C137A mutation, N167I mutation and S168G mutation, wherein the positions of mutations are defined in relation to SEQ ID NO: 1.

In some embodiments, the VEGF-C mutein protein or functional fragment thereof comprises C137A mutation, N167I mutation and L192I mutation, wherein the positions of mutations are defined in relation to SEQ ID NO: 1.

In some embodiments, the VEGF-C mutein protein or functional fragment thereof comprises C137A mutation, N167I mutation, S168G mutation, and L192I mutation, wherein the positions of mutations are defined in relation to SEQ ID NO: 1.

In some embodiments, the VEGF-C mutein protein or functional fragment thereof comprises C137A mutation and S168G mutation, wherein the positions of mutations are defined in relation to SEQ ID NO: 1.

In some embodiments, the VEGF-C mutein protein or functional fragment thereof comprises C137A mutation, S168G mutation and L192I mutation, wherein the positions of mutations are defined in relation to SEQ ID NO: 1.

In some embodiments, the VEGF-C mutein protein or functional fragment thereof comprises C137A mutation and N167H mutation, wherein the positions of mutations are defined in relation to SEQ ID NO: 1.

In some embodiments, the VEGF-C mutein protein or functional fragment thereof comprises C137A mutation, N167I mutation and S168R mutation, wherein the positions of mutations are defined in relation to SEQ ID NO: 1.

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the polypeptide sequence of any of the SEQ ID NOs: 50-55, SEQ ID NOs: 60-61, SEQ ID NOs: 66-67, SEQ ID NO: 69, SEQ ID NOs: 71-79, SEQ ID NOs: 81-87, or SEQ ID NOs: 89-99.

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5%, or 100% sequence identity to any of SEQ ID NOs: 102-107, SEQ ID NOS: 112-113, SEQ ID NOs: 118-119, SEQ ID NO: 121, SEQ ID NOs: 123-131, SEQ ID NOs: 133-139, or SEQ ID NOs: 141-151.

The VEGF-C muteins or fragments thereof described herein include variants having single or multiple amino acid substitutions, deletions, or additions that retain the biological properties (e.g., binding affinity or immune effector activity) of the described VEGF-C muteins or fragments thereof.

These variants may include: (i) variants in which one or more amino acid residues are substituted with conservative or nonconservative amino acids, (ii) variants in which one or more amino acids are added to or deleted from the polypeptide, (iii) variants in which one or more amino acids include a substituent group, and (iv) variants in which the described VEGF-C mutein or fragment thereof is fused or conjugated with another peptide or polypeptide (e.g., a fusion partner, a protein tag) or other chemical moiety, that may confer useful properties to the VEGF-C mutein or fragment thereof, such as, for example, an epitope for an antibody, a polyhistidine sequence, a biotin moiety and the like. VEGF-C muteins or fragments thereof described herein may include variants in which amino acid residues from one species are substituted for the corresponding residue in another species, either at the conserved or nonconserved positions. In other embodiments, amino acid residues at nonconserved positions are substituted with conservative or nonconservative residues. Amino acid substitutions are generally based on the relative similarity of the amino acid side-chain substituents, for example, their hydrophobicity, hydrophilicity, charge, size, and the like.

Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group I (Ala, Ser, Thr, Pro, Gly); Group II (Asp, Asn, Glu, Gln); Group III (His, Arg, Lys); Group IV (Met, Leu, Ile, Val, Cys); Group V (Phe, Thy, Trp).

In some embodiments, the VEGF-C mutein protein is modified to extend its circulating half-life. Strategies to extend the half-life of recombinant proteins include, but are not limited to fusion to immunoglobulin of a fragment thereof of immunoglobulin such as the Fc domain of IgG; fusion to albumin or an albumin fragment thereof; fusion to an albumin-binding antibody or antibody fragment thereof such as an scFv, Fab, or single-domain antibody (VHH); or chemical modification with polyethylene glycol (PEG).

In some embodiments, the VEGF-C mutein, or a fragment thereof or a variant thereof, is fused to and/or conjugated to one or more heterologous moieties, such as, but not limited to, peptides, polypeptides, small molecules, polymers, nucleic acids, lipids, sugars, etc.

In certain embodiments, the VEGF-C muteins are fused to and/or conjugated to a moiety that provides longer half-life to the VEGF-C mutein. In some embodiments, the VEGF-C muteins are fused to and/or conjugated to a moiety that specifically binds albumin including but not limited to, a small molecule, a peptide, a polypeptide, or a lipid that bind albumin. In some embodiments, the VEGF-C muteins are fused to and/or conjugated to an immunoglobulin constant region (an Fc domain), an scFv, an Fab, a single-domain antibody (VHH), an immunoglobulin, or a heavy or light chain thereof.

In certain embodiments, the VEGF-C muteins are fused to and/or conjugated to a polymer including, but not limited to, lipid polymers, polyethylene glycol (PEG), biodegradable polymers such as PLA (poly (lactic acid)) and PLGA (poly (lactic-glycolic acid)), polysaccharides, polysaccharides, poly (propylene glycol), polyoxy ethylated polyols, polyvinyl ethers, copolymers of ethylene glycol and propylene glycolpolyvinyl alcohols, dextran, hyaluronic acid, chitin, and the like.

In certain embodiments, the VEGF-C muteins are fused to and/or conjugated to an Fc domain. In certain embodiments, the Fc sequence to be fused or conjugated to the VEGF-C muteins of the present invention may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

(SEQ ID NO: 21)
EPKSCDKTHTCPPCPAPELLGGPSVFLFPPKPKDTLMISRTPEVTCVVVDVSHEDPEVKF
NWYVDGVEVHNAKTKPREEQYQSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK.

In certain embodiments, the polynucleotide molecule encoding the Fc sequence may comprise a sequence with at least or at most 70%, 80%, 85%, 86%, 87%, 88%, 89%, 90%, 91%, 92%, 93%, 94%, 95%, 96%, 97%, 98%, 99% or 100% sequence identity to the following nucleotide sequence:

(SEQ ID NO: 22)
ACTATCAGCTTTGCTAACCATACAAGCTGTCGCTGTATGAGCAAACTGgcggccgcTGA
ACCCAAAAGTTGTGACAAAACGCACACATGCCCGCCTTGCCCCGCCCCGGAGCTAC
TGGGCGGACCCTCTGTGTTTCTGTTTCCACCAAAGCCGAAAGATACTCTTATGATTTC
CAGGACACCTGAAGTCACCTGCGTTGTTGTGGATGTTAGCCATGAAGATCCCGAAGT
GAAGTTTAACTGGTACGTAGACGGCGTGGAAGTACACAATGCAAAAACGAAACCTA
GAGAAGAACAATATCAATCAACCTATAGGGTAGTGTCAGTCCTTACAGTCCTACACC
AGGACTGGCTTAATGGGAAAGAGTATAAATGCAAGGTCAGTAACAAGGCGCTACCC
GCTCCAATAGAGAAAACTATCTCTAAGGCTAAAGGCCAGCCGCGTGAGCCCCAGGT
ATATACACTGCCACCATCCAGGGAGGAAATGACAAAGAACCAGGTGTCCCTGACTT
GCTTAGTAAAAGGGTTTTATCCATCCGACATCGCCGTGGAATGGGAATCTAATGGTC
AGCCTGAAAACAATTACAAGACAACACCTCCCGTTCTTGATAGCGACGGATCTTTTT
TCCTTTACTCTAAATTGACTGTAGATAAAAGTAGGTGGCAGCAAGGTAACGTCTTTT
CATGCAGCGTGATGCACGAGGCATTACACAACCATTACACACAGAAATCATTGTCAT
TAAGTCCAGGGAAG.

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGAATNTFFKPPCVSVYRCGGCCNSEGLQC
MNTSTGYLSKTLFEITVPLSQGPKPVTISFANHTSCRCMSKLAAAEPKSCDKTHTCPPCP
APELLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK (murine VEGF-C mutein
protein with C133A mutation and Fc conjugation, SEQ ID NO: 11)

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99%, or 100% sequence identity to the following

AACACGGAAATCCTCAAGTCTATAGATAATGAGTGGCGAAAGACACAATGTATGCC
GCGCGAGGTAGCAATCGACGTTGGTAAAGAATTTGGTGCGGCAACGAACACATTCT
TCAAACCCCCTTGTGTGAGTGTATATAGATGTGGAGGGTGTTGCAATTCTGAGGGGC
TCCAATGTATGAACACGAGTACGGGTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAG (nucleotide
sequence encoding murine VEGF-C mutein protein with C133A mutation
and Fc conjugation, SEQ ID NO: 12).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGAATNTFFKPPCVSVYRCGGCCRSEGLQC
MNTSTGYLSKTLFEITTPISQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK (murine VEGF-C mutein
protein with C133A, N163R, V186T, and L188I mutations with Fc conjugation,
SEQ ID NO: 15).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99%, or 100% sequence identity to the following

AACACCGAAATCTTGAAATCAATAGATAACGAATGGCGAAAAACCCAATGTATGCC
ACGCGAGGTAGCGATAGATGTGGGCAAAGAATTCGGCGCCGCGACGAACACCTTTT
TCAAGCCCCCTTGCGTCTCCGTATATAGATGCGGTGGATGTTGCCGATCCGAGGGCC
TTCAGTGTATGAACACATCTACTGGCTATTTGAGCAAGACGCTCTTTGAGATTACAA
CACCAATTAGTCAAGGTCCCAAGCCTGTTACCATCTCTTTCGCTAACCACACTTCAT
GCCGCTGTATGAGTAAGTTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAG (nucleotide
sequence encoding murine VEGF-C mutein protein with C133A, N163R,
V186T, and L188I mutations with Fc conjugation, SEQ ID NO: 16).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

NTEILKSIDNEWRKTQCMPREVAIDVGKEFGAATNTFFKPPCVSVYRCGGCCISEGLQC
MNTSTGYLSKTLFEITVPLSQGPKPVTISFANHTSCRCMSKLAAAEPKSCDKTHTCPPCP
APELLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREPQ
VYTLPPSREEMTKNQVSLTCLVKGFYPSDIA VEWESNGQPENNYKTTPPVLDSDGSFFL
YSKLTVDKSRWQQGNVFSCSVMHEALHNHYTQKSLSLSPGK (murine VEGF-C mutein
protein with C133A and N163I mutations with Fc conjugation, SEQ ID NO: 19).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99% or 100% sequence identity to the following

AACACCGAAATCTTGAAATCAATAGATAACGAATGGCGCAAAACTCAATGTATGCC
ACGGGAGGTTGCAATAGACGTGGGAAAGGAATTTGGCGCCGCCACGAATACCTTTT
TCAAGCCTCCCTGTGTGAGTGTTTACAGATGTGGTGGTTGTTGCATATCAGAGGGAT
TGCAGTGCATGAACACAAGTACAGGTTACTTGAGTAAAACATTGTTTGAAATCACAG
TACCATTGTCCCAAGGCCCTAAGCCGGTTACGATCTCTTTCGCCAATCATACGTCAT
GCCGCTGTATGAGTAAGTTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAG (nucleotide
sequence encoding murine VEGF-C mutein protein with C133A and N163I
mutations with Fc conjugation, SEQ ID NO: 20).

In some embodiments, the IgG Fc domain comprises the amino acid sequence of SEQ ID NO: 21. In some embodiments, the VEGF-C mutein protein comprises the amino acid sequence of SEQ ID NO: 11. In some embodiments, the VEGF-C mutein comprises the amino acid sequence of SEQ ID NO: 19. In some embodiments, the VEGF-C mutein consists of the amino acid sequence of SEQ ID NO: 19. In some embodiments, the VEGF-C mutein comprises the amino acid sequence of SEQ ID NO: 15. In some embodiments, the VEGF-C mutein consists of the amino acid sequence of SEQ ID NO: 15

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCNSEGLQC
MNTSTSYLSKTLFEITVPLSQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A
VEGF-C protein with Fc conjugation; SEQ ID NO: 266).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCAATTCTGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A VEGF-C protein
with Fc conjugation; SEQ ID NO: 279).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQSEGLQC
MNTSTSYLSKTLFEITVPLSQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A and
N167Q VEGF-C mutein protein with Fc conjugation; SEQ ID NO: 267).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCAGTCTGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A and N167Q VEGF-C
mutein protein with Fc conjugation; SEQ ID NO: 280).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQGEGLQC
MNTSTSYLSKTLFEITVPLSQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A,
N167Q and S168G VEGF-C mutein protein with Fc conjugation; SEQ ID NO: 268).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCAGGGCGAGGGG
CTCCAATGTATGAACACGAGTACGTCTTACTTGAGTAAGACCTTGTTCGAAATTACA
GTACCACTTTCCCAAGGACCGAAGCCTGTGACTATCAGCTTTGCTAACCATACAAGC
TGTCGCTGTATGAGCAAACTGGCGGCCGCTGAACCCAAAAGTTGTGACAAAACGCA
CACATGCCCGCCTTGCCCCGCCCCGGAGCTACTGGGCGGACCCTCTGTGTTTCTGTTT
CCACCAAAGCCGAAAGATACTCTTATGATTTCCAGGACACCTGAAGTCACCTGCGTT
GTTGTGGATGTTAGCCATGAAGATCCCGAAGTGAAGTTTAACTGGTACGTAGACGGC
GTGGAAGTACACAATGCAAAAACGAAACCTAGAGAAGAACAATATCAATCAACCTA
TAGGGTAGTGTCAGTCCTTACAGTCCTACACCAGGACTGGCTTAATGGGAAAGAGTA
TAAATGCAAGGTCAGTAACAAGGCGCTACCCGCTCCAATAGAGAAAACTATCTCTA
AGGCTAAAGGCCAGCCGCGTGAGCCCCAGGTATATACACTGCCACCATCCAGGGAG
GAAATGACAAAGAACCAGGTGTCCCTGACTTGCTTAGTAAAAGGGTTTTATCCATCC
GACATCGCCGTGGAATGGGAATCTAATGGTCAGCCTGAAAACAATTACAAGACAAC
ACCTCCCGTTCTTGATAGCGACGGATCTTTTTTCCTTTACTCTAAATTGACTGTAGAT
AAAAGTAGGTGGCAGCAAGGTAACGTCTTTTCATGCAGCGTGATGCACGAGGCATT
ACACAACCATTACACACAGAAATCATTGTCATTAAGTCCAGGGAAGCACCATCACC
ATCACCAT (nucleotide sequence encoding human C137A, N167Q and S168G
VEGF-C mutein protein with Fc conjugation; SEQ ID NO: 281).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQSEGLQC
MNTSTSYLSKTLFEITVPISQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A,
N167Q and L192I VEGF-C mutein protein with Fc conjugation; SEQ ID NO: 269).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCAGTCTGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A, N167Q and L192I VEGF-C
mutein protein with Fc conjugation; SEQ ID NO: 282).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCQGEGLQC
MNTSTSYLSKTLFEITVPISQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A, N167Q,
S168G and L192I VEGF-C mutein protein with Fc conjugation; SEQ ID NO: ).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCAGGGCGAGGGG
CTCCAATGTATGAACACGAGTACGTCTTACTTGAGTAAGACCTTGTTCGAAATTACA
GTACCAATCTCCCAAGGACCGAAGCCTGTGACTATCAGCTTTGCTAACCATACAAGC
TGTCGCTGTATGAGCAAACTGGCGGCCGCTGAACCCAAAAGTTGTGACAAAACGCA
CACATGCCCGCCTTGCCCCGCCCCGGAGCTACTGGGCGGACCCTCTGTGTTTCTGTTT
CCACCAAAGCCGAAAGATACTCTTATGATTTCCAGGACACCTGAAGTCACCTGCGTT
GTTGTGGATGTTAGCCATGAAGATCCCGAAGTGAAGTTTAACTGGTACGTAGACGGC
GTGGAAGTACACAATGCAAAAACGAAACCTAGAGAAGAACAATATCAATCAACCTA
TAGGGTAGTGTCAGTCCTTACAGTCCTACACCAGGACTGGCTTAATGGGAAAGAGTA
TAAATGCAAGGTCAGTAACAAGGCGCTACCCGCTCCAATAGAGAAAACTATCTCTA
AGGCTAAAGGCCAGCCGCGTGAGCCCCAGGTATATACACTGCCACCATCCAGGGAG
GAAATGACAAAGAACCAGGTGTCCCTGACTTGCTTAGTAAAAGGGTTTTATCCATCC
GACATCGCCGTGGAATGGGAATCTAATGGTCAGCCTGAAAACAATTACAAGACAAC
ACCTCCCGTTCTTGATAGCGACGGATCTTTTTTCCTTTACTCTAAATTGACTGTAGAT
AAAAGTAGGTGGCAGCAAGGTAACGTCTTTTCATGCAGCGTGATGCACGAGGCATT
ACACAACCATTACACACAGAAATCATTGTCATTAAGTCCAGGGAAGCACCATCACC
ATCACCAT (nucleotide sequence encoding human C137A, N167Q, S168G and L192I
VEGF-C mutein protein with Fc conjugation; SEQ ID NO: 283).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCISEGLQC
MNTSTSYLSKTLFEITVPLSQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A and
N167I VEGF-C mutein protein with Fc conjugation; SEQ ID NO: 271).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCTCTGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A and N167I VEGF-C
with Fc conjugation; SEQ ID NO: 284).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCIGEGLQC
MNTSTSYLSKTLFEITVPLSQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A,
N167I and S168G VEGF-C mutein protein with Fc conjugation; SEQ ID NO: 272).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCGGCGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A, N167I and S168G
VEGF-C mutein protein with Fc conjugation; SEQ ID NO: 285).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCISEGLQC
MNTSTSYLSKTLFEITVPISQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A,
N167I and L192I VEGF-C mutein protein with Fc conjugation; SEQ ID NO: 273).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCTCTGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A, N167I and L192I VEGF-C mutein
protein with Fc conjugation; SEQ ID NO: 286).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCIGEGLQC
MNTSTSYLSKTLFEITVPISQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A,
N167I, S168G and L192I VEGF-C mutein protein with Fc conjugation; SEQ ID NO: 274).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCGGCGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A, N167I, S168G and L192I
VEGF-C mutein protein with Fc conjugation; SEQ ID NO: 287).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCNGEGLQC
MNTSTSYLSKTLFEITVPLSQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A and
S168G VEGF-C mutein protein with Fc conjugation; SEQ ID NO: 275).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCAATGGCGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A and S168G VEGF-C mutein
protein with Fc conjugation; SEQ ID NO: 288).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCNGEGLQC
MNTSTSYLSKTLFEITVPISQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A,
S168G and L192I VEGF-C mutein protein with Fc conjugation; SEQ ID NO: 276).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCAATGGCGAGGGGC
TCCAATGTATGAACACGAGTACGTCTTACTTGAGTAAGACCTTGTTCGAAATTACAG
TACCAATCTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A, S168G and L192I
VEGF-C mutein protein with Fc conjugation; SEQ ID NO: 289).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCHSEGLQC
MNTSTSYLSKTLFEITVPLSQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A and
N167H VEGF-C mutein protein with Fc conjugation; SEQ ID NO: 277).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCCACTCTGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A and N167H VEGF-C
mutein protein with Fc conjugation; SEQ ID NO: 290).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGVATNTFFKPPCVSVYRCGGCCIREGLQC
MNTSTSYLSKTLFEITVPLSQGPKPVTISFANHTSCRCMSKLAAAEPKSCDKTHTCPPCPA
PELLGGPSVFLFPPKPKDTLMISRTPEVTCVVVDVSHEDPEVKFNWYVDGVEVHNAKTK
PREEQYQSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKHHHHHH (human C137A,
N167I and S168R VEGF-C mutein protein with Fc conjugation; SEQ ID NO: 278).

In certain embodiments, the polynucleotide molecule encoding the VEGF-C mutein may comprise a sequence with at least or at most 90%, 91%, 92%, 93%, 94%, 95%, 96%, 97%, 98%, 99%, 99.5% or 100% sequence identity to the following nucleotide sequence:

AACACGGAAATCCTCAAGTCTATAGATAATGAGTGGCGAAAGACACAATGTATGCC
GCGCGAGGTAGCAATCGACGTTGGTAAAGAATTTGGTGTAGCAACGAACACATTCT
TCAAACCCCCTTGTGTGAGTGTATATAGATGTGGAGGGTGTTGCATCAGAGAGGGGC
TCCAATGTATGAACACGAGTACGTCTTACTTGAGTAAGACCTTGTTCGAAATTACAG
TACCACTTTCCCAAGGACCGAAGCCTGTGACTATCAGCTTTGCTAACCATACAAGCT
GTCGCTGTATGAGCAAACTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAGCACCATCACCA
TCACCAT (nucleotide sequence encoding human C137A, N167I and S168R
VEGF-C mutein protein with Fc conjugation; SEQ ID NO: 291).

In some embodiments, the present invention relates to modifying the IgG Fc domain to reduce a Fc effector function. Such modification can be achieved by various techniques, including, but not limited to, amino acid substitutions, deletions, or additions to the Fc domain. For example, the modification may involve replacing one or more amino acid residues in the Fc domain with non-natural amino acids, altering glycosylation patterns, or introducing steric hindrance to the Fc domain. In some embodiments, the modified Fc domain exhibits reduced binding affinity to Fc receptors or complement proteins, resulting in reduced Fc effector function. Examples of modified Fc domains that exhibit reduced Fc effector function include, but are not limited to, Fc variants with point mutations such as N297A, L234A/L235A, S239D/1332E, S298A/IgG1/IgG3, L309D/1332E, M428L, and N434S, or Fc variants with additional glycosylation sites. These modifications can be used to generate therapeutic antibodies with improved safety profiles and reduced risk of adverse immune reactions. The modified Fc domains may also be used to design antibody-based therapeutics with desired effector functions, such as reduced ADCC, complement activation, or immune complex formation. In some embodiments, the IgG Fc domain is modified to reduce a Fc effector function. In some embodiments, the IgG Fc domain comprises a mutation at residue N297. In some embodiments, the mutation at residue N297 is selected from N297Q, N297A and N297G. In some embodiments, the IgG Fc domain is an IgG4 variant comprising a mutation one or more mutations at residues F234, or L235. In some embodiments, the IgG Fc domain is an IgG4 variant comprising a mutation one or more mutations F234A, or L235A. In some embodiments, the IgG Fc domain is an IgG1 variant comprising a mutation one or more mutations at residues L234, or L235. In some embodiments, the IgG Fc domain is an IgG1 variant comprising one or more mutations L234A, or L235A. In some embodiments, the IgG Fc domain can be a cross-subclass domain. For example, but no limited to, the IgG Fc domain can be IgG2 variant with point mutations from IgG4 (e.g., H268Q/V309L/A330S/P331S).

In some embodiments, the Fc domain comprises one or more mutations. In some embodiments, the Fc domain is mutated, glycoengineered, or otherwise modified to reduce Fc effector functions.

In some embodiments, the Fc domain comprises one or more mutations that ablate a critical glycosylation site required for effector function.

In some embodiments, the Fc domain comprises a mutation at residue N82, wherein the positions of said residues are defined in relation to SEQ ID NO: 21. In some embodiments, the mutation at N82, defined in relation to SEQ ID NO: 21, is N82Q mutation, N82A mutation, or N82G mutation.

In certain embodiments, the VEGF-C muteins are fused to and/or conjugated to albumin. In certain embodiments, the albumin is human albumin.

In some embodiments, VEGF-C muteins are fused to and/or conjugated to albumin or to an Fc domain though a linker. Examples of linkers include, but not limited to, proline-rich linkers, acidic linkers, basic linkers, cleavable linkers or rigid linkers. Examples of proline-rich linkers include, but not limited to, (Pro-Pro-Gly) 3 (SEQ ID NO: 295), (Pro-Gly) 5 (SEQ ID NO: 296), or (Pro-Pro-Pro-Pro-Gly) 3 (SEQ ID NO: 297). Examples of acidic linkers include, but not limited to, (Glu-Ser-Glu-Ser) 3 (SEQ ID NO: 298), (Asp-Glu) 5 (SEQ ID NO: 299), or (Glu-Asp-Glu-Asp-Glu) 3 (SEQ ID NO: 300). Examples of basic linkers include, but not limited to, (Lys-Ser-Lys-Ser) 3 (SEQ ID NO: 301), (Arg-Lys-Arg-Lys) 3 (SEQ ID NO: 302), or (Lys-Arg-Lys-Arg-Lys) 3 (SEQ ID NO: 303). Examples of cleavable linkers include, but not limited to, (Gly-Gly-Ser) 3 (SEQ ID NO: 304), which is cleavable by proteases such as trypsin, chymotrypsin, or thrombin, or (Leu-Val-Pro-Arg) (SEQ ID NO: 305), which is cleavable by the protease factor Xa. Examples of rigid linkers include, but not limited to, (Azido-Lys-Arg-Pro) m which contains the non-natural amino acid azido-lysine, which can form a triazole linkage with an alkyne-containing molecule, or (Cyclohexyl-Ala-Pro-Pro), which contains the non-natural amino acid cyclohexyl-alanine, which can form a rigid cyclohexane structure. Example of such linker, but not limited to, includes a linker with the amino acid sequence: GGGGGSGGGGSGGGGS (SEQ ID NO: 294).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

NTEILKSIDNEWRKTQCMPREVAIDVGKEFGAATNTFFKPPCVSVYRCGGCCISEGLQC
MNTSTGYLSKTLFEITVPLSQGPKPVTISFANHTSCRCMSKLHHHHHHHH (murine
VEGF-C mutein protein with C133A and N163I mutations with 8xHis (SEQ
ID NO: 306) aa sequence; SEQ ID NO: 254).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99%, or 100% sequence identity to the following nucleotide sequence:

AACACCGAAATCTTGAAATCAATAGATAACGAATGGCGCAAAACTCAATGTATGCC
ACGGGAGGTTGCAATAGACGTGGGAAAGGAATTTGGCGCCGCCACGAATACCTTTT
TCAAGCCTCCCTGTGTGAGTGTTTACAGATGTGGTGGTTGTTGCATATCAGAGGGAT
TGCAGTGCATGAACACAAGTACAGGTTACTTGAGTAAAACATTGTTTGAAATCACAG
TACCATTGTCCCAAGGCCCTAAGCCGGTTACGATCTCTTTCGCCAATCATACGTCAT
GCCGCTGTATGAGTAAGTTGCATCACCATCACCATCATCACCAT (nucleotide sequence
encoding murine VEGF-C mutein protein with C133A and N163I mutations
with 8xHis (SEQ ID NO: 306) aa sequence; SEQ ID NO: 260).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGAATNTFFKPPCVSVYRCGGCCRSEGLQC
MNTSTGYLSKTLFEITTPISQGPKPVTISFANHTSCRCMSKLHHHHHHHH (murine VEGF-
C mutein protein with C133A, N163R, V186T and L188I mutations with
8xHis (SEQ ID NO: 306) aa sequence; SEQ ID NO: 255).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99% or 100% sequence identity to the following

AACACCGAAATCTTGAAATCAATAGATAACGAATGGCGAAAAACCCAATGTATGCC
ACGCGAGGTAGCGATAGATGTGGGCAAAGAATTCGGCGCCGCGACGAACACCTTTT
TCAAGCCCCCTTGCGTCTCCGTATATAGATGCGGTGGATGTTGCCGATCCGAGGGCC
TTCAGTGTATGAACACATCTACTGGCTATTTGAGCAAGACGCTCTTTGAGATTACAA
CACCAATTAGTCAAGGTCCCAAGCCTGTTACCATCTCTTTCGCTAACCACACTTCAT
GCCGCTGTATGAGTAAGTTGCATCACCATCACCATCATCACCAT (nucleotide sequence
encoding murine VEGF-C mutein protein with C133A, N163R, V186T and L188I
mutations with 8xHis (SEQ ID NO: 306) aa sequence; SEQ ID NO: 261).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

NTEILKSIDNEWRKTQCMPREVAIDVGKEFGAATNTFFKPPCVSVYRCGGCCISEGLQC
MNTSTGYLSKTLFEITVPLSQGPKPVTISFANHTSCRCMSKLAAAEPKSCDKTHTCPPCP
APELLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK (murine VEGF-C mutein
protein with C133A and N163I mutations AAA cloning scar Fc (N297Q)
fusion 6xHis (SEQ ID NO: 25) aa sequence; SEQ ID NO: 256).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99% or 100% sequence identity to the following

AACACCGAAATCTTGAAATCAATAGATAACGAATGGCGCAAAACTCAATGTATGCC
ACGGGAGGTTGCAATAGACGTGGGAAAGGAATTTGGCGCCGCCACGAATACCTTTT
TCAAGCCTCCCTGTGTGAGTGTTTACAGATGTGGTGGTTGTTGCATATCAGAGGGAT
TGCAGTGCATGAACACAAGTACAGGTTACTTGAGTAAAACATTGTTTGAAATCACAG
TACCATTGTCCCAAGGCCCTAAGCCGGTTACGATCTCTTTCGCCAATCATACGTCAT
GCCGCTGTATGAGTAAGTTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAG (nucleotide
sequence encoding murine VEGF-C mutein protein with C133A and N163I
mutations AAA cloning scar Fc (N297Q) fusion 6xHis (SEQ ID NO: 25) aa
sequence; SEQ ID NO: 262).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGAATNTFFKPPCVSVYRCGGCCISEGLQC
MNTSTGYLSKTLFEITVPLSQGPKPVTISFANHTSCRCMSKLAAAEPKSCDKTHTCPPCP
APELLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK (murine VEGF-C mutein
protein with C133A, N163R, V186T and L188I mutations AAA cloning scar
Fc (N297Q) fusion 6xHis aa sequence; SEQ ID NO: 257).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99%, or 100% sequence identity to the following

AACACCGAAATCTTGAAATCAATAGATAACGAATGGCGCAAAACTCAATGTATGCC
ACGGGAGGTTGCAATAGACGTGGGAAAGGAATTTGGCGCCGCCACGAATACCTTTT
TCAAGCCTCCCTGTGTGAGTGTTTACAGATGTGGTGGTTGTTGCATATCAGAGGGAT
TGCAGTGCATGAACACAAGTACAGGTTACTTGAGTAAAACATTGTTTGAAATCACAG
TACCATTGTCCCAAGGCCCTAAGCCGGTTACGATCTCTTTCGCCAATCATACGTCAT
GCCGCTGTATGAGTAAGTTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAG (nucleotide
sequence encoding murine VEGF-C mutein protein with C133A, N163R, V186T and
L188I mutations AAA cloning scar Fc (N297Q) fusion 6xHis (SEQ ID NO: 25) aa
sequence; SEQ ID NO: 263).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99%, or 100% identical to the following polypeptide sequence:

NTEILKSIDNEWRKTQCMPREVAIDVGKEFGAATNTFFKPPCVSVYRCGGCCISEGLQC
MNTSTGYLSKTLFEITVPLSQGPKPVTISFANHTSCRCMSKLAAAEPKSCDKTHTCPPCP
APELLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK (murine VEGF-C mutein
protein with C133A and N163I mutations AAA cloning scar and linker mouse
serum albumin fusion GS 8xHis (SEQ ID NO: 306) aa sequence; SEQ ID NO: 258).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99% or 100% sequence identity to the following nucleotide sequence:

AACACCGAAATCTTGAAATCAATAGATAACGAATGGCGCAAAACTCAATGTATGCC
ACGGGAGGTTGCAATAGACGTGGGAAAGGAATTTGGCGCCGCCACGAATACCTTTT
TCAAGCCTCCCTGTGTGAGTGTTTACAGATGTGGTGGTTGTTGCATATCAGAGGGAT
TGCAGTGCATGAACACAAGTACAGGTTACTTGAGTAAAACATTGTTTGAAATCACAG
TACCATTGTCCCAAGGCCCTAAGCCGGTTACGATCTCTTTCGCCAATCATACGTCAT
GCCGCTGTATGAGTAAGTTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAG nucleotide
sequence encoding (murine VEGF-C mutein protein with C133A and N163I
mutations AAA cloning scar and linker mouse serum albumin fusion GS 8xHis
(SEQ ID NO: 306) aa sequence; SEQ ID NO: 264).

In certain embodiments, the VEGF-C mutein may comprise a sequence that is at least or at most 70%, 80%, 85%, 86%, 87%, 88%, 89%, 90%, 91%, 92%, 93%, 94%, 95%, 96%, 97%, 98%, 99% or 100% identical to the following polypeptide sequence:

NTEILKSIDNEWRKTQCMPREVAIDVGKEFGAATNTFFKPPCVSVYRCGGCCISEGLQC
MNTSTGYLSKTLFEITVPLSQGPKPVTISFANHTSCRCMSKLAAAEPKSCDKTHTCPPCP
APELLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK (murine VEGF-C mutein
protein with C133A, N163R, V186T and L188I mutations AAA cloning scar
and linker mouseserum albumin fusion GS 8xHis (SEQ ID NO: 306) aa
sequence; SEQ ID NO: 259).

In certain embodiments, the polynucleotide molecule encoding the VEGF-C mutein may comprise a sequence with at least or at most 70%, 80%, 85%, 86%, 87%, 88%, 89%, 90%, 91%, 92%, 93%, 94%, 95%, 96%, 97%, 98%, 99%, or 100% sequence identity to the following nucleotide sequence:

AACACCGAAATCTTGAAATCAATAGATAACGAATGGCGCAAAACTCAATGTATGCC
ACGGGAGGTTGCAATAGACGTGGGAAAGGAATTTGGCGCCGCCACGAATACCTTTT
TCAAGCCTCCCTGTGTGAGTGTTTACAGATGTGGTGGTTGTTGCATATCAGAGGGAT
TGCAGTGCATGAACACAAGTACAGGTTACTTGAGTAAAACATTGTTTGAAATCACAG
TACCATTGTCCCAAGGCCCTAAGCCGGTTACGATCTCTTTCGCCAATCATACGTCAT
GCCGCTGTATGAGTAAGTTGGCGGCCGCTGAACCCAAAAGTTGTGACAAAACGCAC
ACATGCCCGCCTTGCCCCGCCCCGGAGCTACTGGGCGGACCCTCTGTGTTTCTGTTTC
CACCAAAGCCGAAAGATACTCTTATGATTTCCAGGACACCTGAAGTCACCTGCGTTG
TTGTGGATGTTAGCCATGAAGATCCCGAAGTGAAGTTTAACTGGTACGTAGACGGCG
TGGAAGTACACAATGCAAAAACGAAACCTAGAGAAGAACAATATCAATCAACCTAT
AGGGTAGTGTCAGTCCTTACAGTCCTACACCAGGACTGGCTTAATGGGAAAGAGTAT
AAATGCAAGGTCAGTAACAAGGCGCTACCCGCTCCAATAGAGAAAACTATCTCTAA
GGCTAAAGGCCAGCCGCGTGAGCCCCAGGTATATACACTGCCACCATCCAGGGAGG
AAATGACAAAGAACCAGGTGTCCCTGACTTGCTTAGTAAAAGGGTTTTATCCATCCG
ACATCGCCGTGGAATGGGAATCTAATGGTCAGCCTGAAAACAATTACAAGACAACA
CCTCCCGTTCTTGATAGCGACGGATCTTTTTTCCTTTACTCTAAATTGACTGTAGATA
AAAGTAGGTGGCAGCAAGGTAACGTCTTTTCATGCAGCGTGATGCACGAGGCATTA
CACAACCATTACACACAGAAATCATTGTCATTAAGTCCAGGGAAG (nucleotide
sequence encoding murine VEGF-C mutein protein with C133A, N163R, V186T
and L188I mutations AAA cloning scar and linker mouse serum albumin fusion
GS 8xHis (SEQ ID NO: 306) aa sequence; SEQ ID NO: 265).

In some embodiments, the polynucleotide molecule encoding the VEGF-C mutein or the functional fragment thereof is a mRNA.

mRNA can provide several advantages to AAV and other gene delivery systems, which include, but are not limited to one or more of the following: mRNA can be highly customizable, mRNA can prevent recognition from pattern recognition receptors and nucleases to allow for sustained expression, mRNA can provide for well-controlled expression kinetics with the option of repeated dosing, and mRNA can provide for low risk of integration into the genome due to its localization in the cytosol. mRNA can also be cost-effective.

In some embodiments, the polynucleotide molecule encoding the VEGF-C mutein protein or the functional fragment thereof comprises a modified nucleotide such as 5-methyl-cytosine and pseudo-uridine substitutions that can increase stability, decrease deamination, decrease nuclease activity, decrease innate recognition, or increase translation efficiency of the polynucleotide molecule. In some embodiments, the modified nucleotide is a 5-methyl-cytosine or a pseudo-uridine. In some embodiments, the polynucleotide molecule encoding the VEGF-C mutein comprises a 5′ cap.

The mRNA may comprise a modified nucleotide. In some embodiments, the modified nucleotide is a 5-methyl-cytosine or a pseudo-uridine. In some embodiments, the polynucleotide molecule encoding the VEGF-C mutein or the functional fragment thereof comprises a 5′ cap. In some embodiments, the 5′ cap is added using the CleanCap Reagent AG. CleanCap is made up of C32H43N15024P4 and allows for high capping efficiencies resulting in more active mRNA. Cap 1 does not activate Pattern Recognition Receptors and is important for proficient in vivo expression. Without wishing to be bound by theory, any one or more of 5-methyl-cytosine, pseudo-uridine, and the 5′ cap may improve stability of the mRNA, which in turn can prolong expression of the VEGF-C mutein or the functional fragment thereof.

In some embodiments, the polynucleotide molecule encoding the VEGF-C mutein or the functional fragment thereof is comprised within a viral vector. Exemplary viral vectors include, but are not limited to, herpes virus, cytomegalovirus, poliovirus, alphavirus, vaccinia virus, rabies virus, adeno-associated virus (AAV), a retrovirus, a lentivirus, and adenovirus. The retrovirus may be a lentivirus. The recombinant viral particle may be derived from an adeno-associated virus (AAV). In some embodiments, the AAV is AAV2. In some embodiments, the AAV is AAV5. In some embodiments, the AAV is AAV9.

In some embodiments, the VEGF-C mutein or the functional fragment thereof can be administered in a dosage regimen involving a combination of mRNA and AAV. One or more administrations of mRNA can be undertaken to quickly obtain high expression, such as within 2 hours post delivery of the mRNA. The expression of VEGF-C mutein or the functional fragment thereof provided by AAV may take 7-14 days, or even up to four weeks, depending on the serotype. Administration of protein or the functional fragment thereof can be undertaken to get instantaneous expression. Administration of both mRNA and AAV, conjointly or in short succession, can provide a sustained expression of the VEGF-C mutein or the functional fragment thereof. Without wishing to be bound by theory, administering protein can provide for instantaneous expression and controlled expression kinetics, with multiple doses possible. Without wishing to be bound by theory, administering mRNA can provide for instantaneous expression, controlled expression kinetics, and high expression, with multiple doses possible. Without wishing to be bound by theory, administering AAV can provide for delayed expression and high levels of expression. The expression kinetics can be effectively and sensitively measured using ELISA and Western blotting.

In some embodiments, the polynucleotide molecule encoding the VEGF-C mutein or the functional fragment thereof is comprised within a liposome. The VEGF-C mutein or the functional fragment thereof may be encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The liposome comprising the VEGF-C mutein or the functional fragment thereof may be present in a bilayer structure, as micelles, or with a “collapsed” structure. The liposomes may also simply be interspersed in a solution, possibly forming aggregates which are not uniform in either size or shape. For example, a nucleotide (e.g., siRNA) may be encapsulated in a neutral liposome using a method involving ethanol and calcium. The shape may be that of a spherical vesicle. In various embodiments, the liposomes may comprise one or more concentric layers of lipid bilayer molecules. In some embodiments, the lipid components include a combination of Cl 2-200, XTC, MC3, NC98-5, DLinDMA, HGT5001cis, HGT5001trans, HGT5000, HGT4003, DLinKC2DMA, ALNY100, ICE, DLinKC2DMA, CHOL, DOPE, DMG-PEG-2000, Cl 2-200, DOPE, CHOL, and DMGPEG2K.

In some embodiments, the polynucleotide molecule encoding the VEGF-C mutein or the functional fragment thereof is attached to a nanoparticle or a polymer. In certain embodiments, present nanoparticles further comprise at least one agent that specifically binds a particular type or category of cells and/or other particular type compounds, (e.g., a moiety that targets a specific ceil or type of cell). In some embodiments, the nanoparticle is a nanosphere. In some embodiments, the polymer is dextran, poly (amine-co-ester), poly (beta-amino-ester), polyethylenimine, poly-L-Lysine, polyethylene glycol, or dendrimers.

In some embodiments, the polynucleotide molecule encoding the VEGF-C mutein or the functional fragment thereof is comprised within a recombinant viral particle or within a virus like particle (VLP).

In some embodiments, the VEGF-C mutein is produced and administered as a “masked” prodrug that is activated after administration to a patient. In some cases, this may confer desirable tissue or tumor-specific activity of the mutein, for example by virtue of restricted expression of endogenous proteases that cleave the prodrug and release an active “mature” fragment. In some embodiments, the VEGF-C mutein is a variant of the full-length VEGF-C propeptide and activated by proteases such as, e.g., ADAMTS2, plasmin, furin, Cathepsin D, thrombin, and/or KLK3/PSA. In other embodiments, the VEGF-C mutein is fused to an inhibitory peptide that is released by proteolysis by an endogenous protease. The inhibitory peptide can take many forms, including by not limited to, an antibody or antibody fragment that binds to VEGF-C mutein to obscure its interaction with VEGFR-3, the VEGFR-3 ectodomain or fragment thereof that competitively inhibits interaction of VEGF-C with the VEGFR-3-on cells, and any other binder (such as, e.g., a VHH, fibronectin domain, knottin, lipocalin, leucine-rich repeat, etc). A non-limiting set of examples of protease cleavage sites is described below. The inhibitory peptide may be separated from the biologically active VEGF-C mutein by an enterokinase cleavage site (EKCS) peptide, which is recognized and cleaved by the endogenous protease enterokinase present in the small intestine. In this way, the prodrug can be designed to be inactive until it reaches the small intestine, where it can be activated by enterokinase cleavage. Alternatively, the inhibitory peptide can be separated from the biologically active VEGF-C mutein by a furin cleavage site (FCS) peptide that is recognized and cleaved by furin, an endogenous protease that is overexpressed in many tumor cells. By designing the prodrug to be cleaved specifically by furin, the activation of the prodrug can be localized to the tumor microenvironment, resulting in targeted therapy. Furthermore, the prodrugs can be designed to be cleaved by other endogenous proteases that are overexpressed in specific tumors. For example, the prodrug can be designed to be cleaved by matrix metaloproteases MMPs or urokinase-type plasminogen activator uPA, which are overexpressed in many tumors. In this way, the activation of the prodrug can be localized to the tumor microenvironment, resulting in a targeted VEGF-C mutein therapy.

In one aspect is provided a method of inducing lymphangiogenesis in a subject in need thereof, the method comprising administering to the subject an effective amount of a VEGF-C mutein protein or a functional fragment thereof, or a fusion protein or conjugate thereof, or a polynucleotide molecule encoding said VEGF-C mutein protein or functional fragment thereof or fusion protein thereof, or a vector or particle comprising said polynucleotide molecule, or a pharmaceutical composition comprising any of the above.

In one aspect, provided herein is a method of treating a disease or condition in a subject in need thereof, the method comprising administering to the subject an effective amount of a VEGF-C mutein proteins or functional fragments thereof of the present disclosure or the fusion proteins of the present disclosure, or the polynucleotide molecules of the present disclosure, or the vectors of the present disclosure, or the particles of the present disclosure, or the pharmaceutical compositions of the present disclosure.

In some embodiments, the disease or condition is cancer, coronary vessel function, osmoregulation, heart ischemia, restenosis, fibrosis, colitis, chronic liver disease, polycystic kidney disease, diseases or conditions associated with lymph node transplant, Alzheimer's disease, Parkinson's disease, stroke, cerebral ischemia, wound healing, lymphedema, Hennekam syndrome, Milroy's disease, Turner syndrome, age related macular degeneration, glaucoma, central serous chorioretinopathy, diabetic retinopathy, macular edema and retinal edema.

In some embodiments, the cancer is in the brain or the central nervous system of the subject. In some embodiments, the cancer is selected from glioma, ependymoma, subependymoma, primitive neuroectodermal tumor, ganglioglioma, Schwannoma, germinoma, craniopharyngioma, meningioma, CNS lymphoma, pineal tumor, retinoblastoma, uveal melanoma and rhabdoid tumor.

In some embodiments, the VEGF-C mutein protein or functional fragment thereof, fusion protein, conjugate, polynucleotide molecule, vector, particle, or pharmaceutical composition is administered intrathecally, intraocularly, intratumorally, intracisternally, intravitreally, via eye drops, subcutaneously, intradermally, via inhalation, via long-dwelling catheter, orally, topically, or systemically. In some embodiments, the pharmaceutical composition is formulated for intrathecal administration. In some embodiments, the pharmaceutical composition is formulated for intratumoral administration. In some embodiments, the pharmaceutical composition is formulated for systemic administration. In some embodiments, the pharmaceutical composition is formulated for intracisternal administration. In some embodiments, the pharmaceutical composition is formulated for eye-drop administration. In some embodiments, the pharmaceutical composition is formulated for intraocular administration. In some embodiments, the pharmaceutical composition is formulated for subcutaneous administration. In some embodiments, the pharmaceutical composition is formulated for intradermal administration. In some embodiments, the pharmaceutical composition is formulated for administration by inhalation. In some embodiments, the pharmaceutical composition is formulated for administration via long-dwelling catheters. In some embodiments, the pharmaceutical composition is formulated for oral administration. In some embodiments, the pharmaceutical composition is formulated for topical administration (e.g., as a cream or gel).

In one aspect is provided a method of treating a cancer in a subject in need thereof, the method comprising administering to the subject an effective amount of a VEGF-C mutein protein or a functional fragment thereof, or a fusion protein or conjugate thereof, or a polynucleotide molecule encoding said VEGF-C mutein protein or functional fragment thereof or fusion protein thereof, or a vector or particle comprising said polynucleotide molecule, or a pharmaceutical composition comprising any of the above.

In one aspect is provided a pharmaceutical composition comprising a VEGF-C mutein or a functional fragment thereof and optionally an immunotherapeutic agent.

In one aspect is provided a method of generating a library of VEGF-C muteins or functional fragments thereof, wherein the VEGF-C muteins or functional fragments thereof have (i) specific binding to VEGF; and (ii) reduced binding to VEGFR-2 compared to the wild-type VEGF-C.

In certain embodiments, the cancer is a melanoma. In certain embodiments the cancer is in the brain or the central nervous system of the subject. Examples of such cancer include, but are not limited to, glioma (e.g., astrocytoma, glioblastoma, oligodendroglioma, brain stem glioma, juvenile pilocytic astrocytoma, and optic nerve glioma), ependymoma, subependymoma, primitive neuroectodermal tumor, ganglioglioma, Schwannoma, germinoma, craniopharyngioma, meningioma, CNS lymphoma, pineal tumor, retinoblastoma, uveal melanoma, and rhabdoid tumor.

The glioma can be any tumor that arises from the glia tissue of the brain. In some embodiments the glioma can be a mixed glioma. The glioma can be a low grade glioma or high grade glioma. The glioma can be supratentorial, infratentorial, or pontine. Examples of glioma include, but are not limited to, glioblastoma.

In certain embodiments, the cancer is glioblastoma. In certain embodiments, the cancer is glioblastoma multiforme (GBM). An initial diagnosis of GBM is generally made using CT or MRI, in which the glioblastomas generally appear as ring-enhancing lesions. Confirmation of the diagnosis can be made based on a biopsy, e.g., a stereotactic biopsy or a craniotomy with tumor resection.

In certain embodiment, the cancer is a metastatic cancer. In certain embodiment, the cancer is a metastatic cancer that has spread into the brain or the central nervous system of the subject. In certain embodiment, the cancer is a metastatic brain cancer.

In certain embodiment, the cancer is melanoma, lung cancer, breast cancer, stomach cancer, esophageal cancer, ovarian cancer, uterine cancer, cervical cancer, head and neck squamous cell carcinoma, thyroid cancer, liquid cancer (such as, e.g., acute myeloid leukemia), kidney cancer, urothelial bladder cancer, prostate cancer, pheochromocytoma, cholangiocarcinoma, liver hepatocellular carcinoma, pancreatic ductal adenocarcinoma, thymoma, sarcoma, mesothelioma, testicular cancer and colorectal cancer.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for the treatment or regulation of cardiac functions or diseases. Examples of such cardiac functions or diseases include, but are not limited to, coronary vessel function, osmoregulation, heart ischemia, and restenosis.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used in pulmonology.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for the treatment of fibrosis.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used in immunology.

In certain, non-limiting, embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for lymph node transplants.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used in gut health. In certain, non-limiting, embodiments, the VEGF-C muteins or the functional fragments thereof can be used for the treatment of colitis and chronic liver disease.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used in nephrology.

In certain, non-limiting, embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for the treatment of polycystic kidney disease.

In certain, non-limiting, embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for the treatment of Alzheimer's disease, Parkinson's disease, stroke, and cerebral ischemia with lung injury. In certain embodiments, the VEGF-C muteins or the functional fragments thereof have neuro-regenerative properties.

In certain, non-limiting, embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for wound healing.

In certain, non-limiting, embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for the treatment of lymphedema. Non-limiting examples of lymphedema include, but are not limited to, primary lymphedema, secondary lymphedema, and hereditary lymphedema.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for the treatment of genetic conditions. Non-limiting examples of such genetic conditions include, but are not limited to, Milroy's disease, Hennekam syndrome and Turner syndrome.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used for the treatment of ocular diseases. Non-limiting examples of such ocular diseases include, but are not limited to, age related macular degeneration, glaucoma, diabetic retinopathy, central serous chorioretinopathy, macular edema, and retinal edema.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used in combination with chimeric antigen receptor (CAR) T cells.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used in combination with cancer vaccines.

In some embodiments, the method does not comprise administering a tumor-specific antigen to the subject.

In certain embodiments, the VEGF-C muteins or the functional fragments thereof of the present disclosure (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be used in combination with an immunotherapeutic agent. In some embodiments, the immunotherapeutic agent is an immune checkpoint inhibitor. The immune checkpoint inhibitor may target PD-1, PD-L1, CTLA-4, TIGIT, TIM-3, LAG-3, BTLA, GITR, 4-1BB, or Ox-40. The immune checkpoint inhibitor may be an anti-PD-1 antibody, an anti-PD-L1 antibody, an anti-CTLA-4 antibody, an anti-TIGIT antibody, an anti-TIM-3 antibody, an anti-LAG-3 antibody, an anti-BTLA antibody, an anti-GITR antibody, an anti-4-IBB antibody, or an anti-Ox-40 antibody. In some embodiments, the immune checkpoint inhibitor is an anti-PD-1 antibody.

The VEGF-C mutein or the functional fragment thereof (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) and the immunotherapeutic agent may be administered conjointly. For example, the VEGF-C mutein and the immunotherapeutic agent are administered in the same composition.

Alternatively, the VEGF-C mutein or the functional fragment thereof (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) and the immunotherapeutic agent may be administered sequentially.

In various embodiments, the VEGF-C mutein or the functional fragment thereof (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) is administered prior to administering the immunotherapeutic agent. The VEGF-C mutein or the functional fragment thereof (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be administered locally to the brain or central nervous system (e.g., to the cisterna magna) and then the immunotherapeutic agent can be administered systemically (e.g., intravenously). The VEGF-C mutein or the functional fragment thereof can be administered intratumorally (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) and then the immunotherapeutic agent can be administered systemically (e.g., intravenously). The VEGF-C mutein or the functional fragment thereof (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be administered intrathecally and then the immunotherapeutic agent can be administered systemically (e.g., intravenously). The VEGF-C mutein or the functional fragment thereof (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above) can be administered directly into the lymphatic system and then the immunotherapeutic agent can be administered systemically (e.g., intravenously).

In various embodiments, the methods for treating cancer further comprise administering an additional anti-cancer treatment to the subject. Examples of the additional anti-cancer treatments include, but are not limited to, surgery, radiation therapy, administration of a chemotherapeutic agent, and any combinations thereof. These additional anti-cancer treatments may be administered before, conjointly with, or after the administration of the VEGF-C mutein or the functional fragment thereof (or fusion proteins or conjugates thereof, or polynucleotide molecules encoding said VEGF-C mutein proteins or functional fragments thereof or fusion proteins thereof, or vectors or particles comprising said polynucleotide molecules, or pharmaceutical compositions comprising any of the above).

In various embodiments, the subject is a human patient. The human patient can be a child or an adult.

In various embodiments, the method is effective to treat the cancer in the subject. In some embodiments, the method is effective to induce lymphangiogenesis in the tumor in the brain or the central nervous system of the subject. In various embodiments, lymphangiogenesis can be confirmed through MRI imaging, e.g., in which the diameter of lymphatic vasculature can be calculated using a contrast agent. In various embodiments, lymphangiogenesis can be confirmed through serial CSF collection to measure VEGFA, VEGFB, VEGFC or VEGFD concentrations. In various embodiments, lymphangiogenesis can be confirmed through serial CSF collection to measure VEGFC concentrations. The method may be effective to reduce tumor volume. In some embodiments, the method is effective to reduce the volume of a tumor in the brain or the central nervous system of the subject. In various embodiments, the method is effective to provide an immune memory against the tumor. Without wishing to be bound by theory, low clinical efficacy of immunotherapy for GBM patients may be due to a low antigen sampling from the CNS at steady state and during initial stages of tumor development. The administration of a VEGF-C mutein or a functional fragment thereof may increase the amount of antigen sampling that occurs in the brain, which in turn could improve the efficacy and outcome of any other immunotherapy (e.g., anti-CTLA-4 antibody) administered. Although VEGFC's role in cancer has been thought to promote metastasis through (lymph) angiogenesis, the inventors have surprisingly shown that in the brain, VEGF-C can reduce tumor size through an increase in immunosurveillance. VEGF-C may stimulate lymphatic endothelial cell proliferation through VEGFR-3 and increase lymphatic vessel functions.

In one aspect, provided herein is a method for modulating intraocular pressure in a subject in need thereof comprising administering to the subject an effective amount of the VEGF-C mutein protein or functional fragment thereof, or a fusion protein or conjugate thereof, or a polynucleotide molecule encoding said VEGF-C mutein protein or functional fragment thereof or fusion protein thereof, or a vector or particle comprising said polynucleotide molecule, or a pharmaceutical composition comprising any of the above, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above.

In one aspect, provided herein is a method for removing unwanted fluid in an eye of a subject in need thereof comprising administering to the subject an effective amount of the VEGF-C mutein protein or functional fragment thereof of the present disclosure, or a fusion protein or conjugate thereof, or a polynucleotide molecule encoding said VEGF-C mutein protein or functional fragment thereof or fusion protein thereof, or a vector or particle comprising said polynucleotide molecule, or a pharmaceutical composition comprising any of the above, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above.

In some embodiments, the unwanted fluid is optic nerve, retinal, subretinal, choroidal, or suprachoroidal fluid.

In some embodiments, wherein the method of the present disclosure is a method for modulating intraocular pressure in a subject in need thereof or a method for removing unwanted fluid in an eye of a subject in need thereof, the subject has glaucoma, macular edema, central serous chorioretinopathy, retinal edema, papilledema, macular degeneration, or diabetic retinopathy.

In some embodiments, wherein the method of the present disclosure is a method for modulating intraocular pressure in a subject in need thereof or a method for removing unwanted fluid in an eye of a subject in need thereof, the VEGF-C mutein protein or functional fragment thereof, fusion protein, conjugate, polynucleotide molecule, vector, particle, or pharmaceutical composition is administered to the posterior eye. In some embodiments, the administration is intraocular. In some embodiments, the intraocular administration is intravitreal, via eye drops, or subretinal.

In one aspect, provided herein is a method for providing neuroprotection in a subject in need thereof comprising administering to the subject an effective amount of the VEGF-C mutein protein or functional fragment thereof of the present disclosure, or a fusion protein or conjugate thereof, or a polynucleotide molecule encoding said VEGF-C mutein protein or functional fragment thereof or fusion protein thereof, or a vector or particle comprising said polynucleotide molecule, or a pharmaceutical composition comprising any of the above, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above. In some embodiments, the neuroprotection is provided, but not limited to, for macular degeneration, glaucoma, stroke, Alzheimer's disease, or Parkinson's disease. In some embodiments, the administration is intraocular. In some embodiments, the intraocular administration is intravitreal, via eye drops, or subretinal.

In one aspect, provided herein is a vaccine comprising the VEGF-C mutein protein or functional fragment thereof of the present disclosure, or a fusion protein or conjugate thereof, or a polynucleotide molecule encoding said VEGF-C mutein protein or functional fragment thereof or fusion protein thereof, or a vector or particle comprising said polynucleotide molecule, or a pharmaceutical composition comprising any of the above.

In one aspect, provided herein is a method inducing an immune response in a subject in need thereof in a subject in need thereof, the method comprising administering to the subject an effective amount of the vaccine of the present disclosure. In some embodiments, the immune response is, but not limited to, an anti-cancer mediated immune response, a vaccine mediated immune response, an anti-viral, immune response, an anti-bacterial immune response or any other anti-pathogen immune response.

### EXAMPLES

The following examples are provided to further describe some of the embodiments disclosed herein. The examples are intended to illustrate, not to limit, the disclosed embodiments.

### Example 1. Generation of VEGF-C Muteins

Therapeutic stimulation of lymph vessel development (lymphangiogenesis) holds promise as an emerging treatment paradigm for a wide range of indications spanning cardiovascular disease to cancer immunotherapy. However, there are currently no specific pharmacologic agents that promote lymphangiogenesis that do not also stimulate potentially harmful angiogenesis. Notably, the principle physiological driver of lymphangiogenesis, Vascular endothelial growth factor C (VEGF-C), agonizes pro-lymphangiogenic signaling via VEGFR-3, but also signals through the pro-angiogenic VEGFR-2. Here, directed evolution with yeast-surface display was used to generate a lymphatic-specific VEGF-C variant that only engages VEGFR-3 but does not signal through VEGFR-2 (LS-VEGF-C). Compared to wild-type VEGF-C, LS-VEGF-C demonstrated superior preclinical efficacy in models of lymphangiogenesis and anti-PD-1 cancer immunotherapy. The biased impact of LS-VEGF-C on lymphangiogenesis versus angiogenesis was further determined herein by studying its effects on augmenting posterior lymphatic drainage in the eye. LS-VEGF-C markedly lowered intraocular pressure in normotensive mice and in two distinct ocular hypertensive mouse models, but without increasing angiogenesis and vascular permeability as seen with WT-VEGF-C treatment. LS-VEGF-C also demonstrated neuroprotective properties independent of intraocular pressure in a model of retinal ganglion cell death from excitotoxic injury. Collectively, these results highlight the feasibility and preclinical efficacy of therapeutic lymphangiogenesis via VEGFR-3-specific agonism and reveal an unexpected impact of lymphangiogenesis in the control of intraocular pressure.

### Protein Expression, Purification and Biotinylation

Extracellular domain of murine VGFR2 (first three Ig domains, amino acids 20-326), VGFR3 (amino acids 25-329), human VGFR2 (amino acids 19-325) and VGFR3 (amino acids 25-329) were cloned into the pEZT vector with an N-terminal H7 signal peptide, a C-terminal AviTag and hexahistidine tag (SEQ ID NO: 25), and expressed by transient transfection of Expi293 cells (Thermo Fisher) per manufacturer's instructions. Proteins were enriched from cell supernatant via Ni-NTA chelating resin and further purified by size exclusion chromatography (Column SEC650, Bio-rad) into a final buffer of HEPES buffered saline (HBS; 10 mM HEPES, pH 7.5, 150 mM NaCl). Protein biotinylation was carried out at room temperature for 2 h with in-house purified BirA ligase enzyme in 0.1 mM bicine (pH 8.3), 10 mM ATP, 10 mM magnesium acetate, and 0.5 mM biotin (Avidity, #BIO500). Biotinylated proteins were then purified by gel-filtration as described above. Biotinylation efficiency was assessed using an SDS/PAGE streptavidin-shift assay.

### Yeast Display of VEGF-C

Murine VEGF-C (amino acids 112-215) was cloned into a C-terminal displayed vector pCT-GCN42 with a yeast GCN4 sequence RMKQLEDKIEELLSKIYHLENEIARLKKLIGER (SEQ ID NO: 26) to promote in-situ dimerization, and displayed on the surface of yeast strain Saccharomyces cerevisiae EBY100. Yeasts were maintained and expanded in liquid synthetic dextrose medium with casamino acids (SDCAA) at 30° C. and then induced for expression in liquid synthetic glucose medium with casamino acids (SGCAA) at 20° C. for 24-48 h. The displayed protein level was verified by staining with a C-terminal Myc tag antibody (Cell Signaling Technology, #3739S). Biotinylated VEGFR-2 and VEGFR-3 binding was detected using a fluorescent streptavidin phycoerythrin secondary and quantified by flow cytometry using a Sony SA3800 flow cytometer.

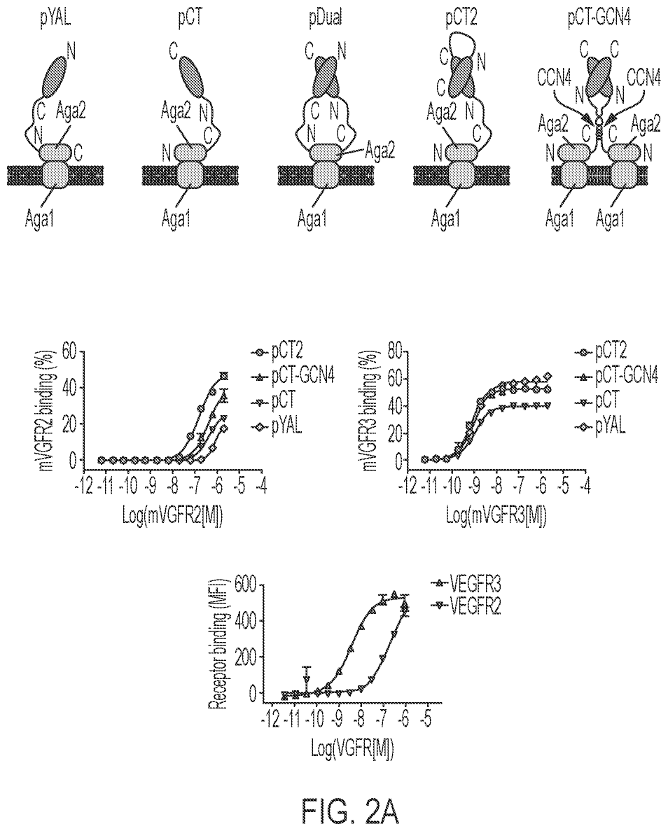

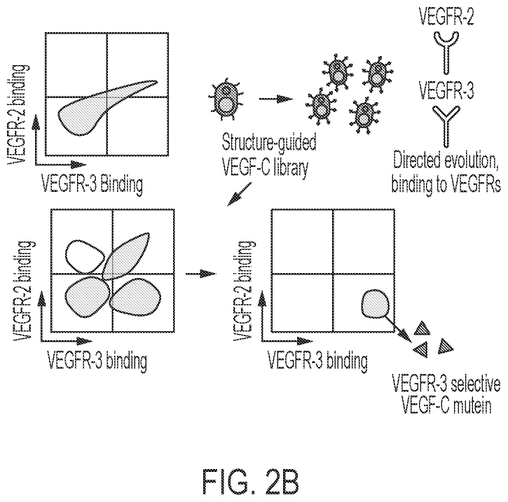

FIG. 2A shows multiple methods to display VEGF-C on yeast surface which were utilized to optimize VEGF-C dimerization on the surface. Final resulting combination displayed wild type VEGF-C with high binding affinity to VEGFR-3 and lower affinity towards VEGFR-2. FIG. 2B is a schematic showing yeast-display library selection towards finding a VEGFR-3 specific VEGF-C mutein.

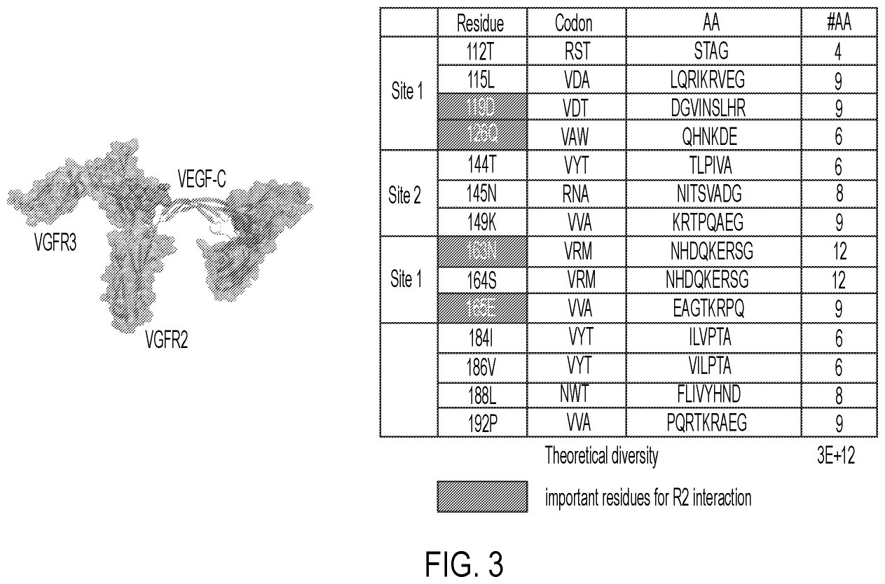

14 residues were identified from analysis of the three dimensional structures of the VEGF-C: VEGFR3 and VEGF-C: VEGFR2 complexes. A library randomizing these residues (FIG. 3) was constructed using assembly PCR. The PCR products were further amplified with primers containing homology for the yeast display vector backbone to enable homologous recombination. Transformed yeast were recovered and expanded in SDCAA medium.

### Mutein Selection

Experiments were conducted using a Biacore T100 and carried out at 25° C. Protein concentrations were quantified by 280 nm absorbance with a Nanodrop2000 spectrometer (Thermo Scientific). Biotinylated proteins, VEGFR-2 or VEGFR-3, were immobilized onto a Biacore biotin CAPture sensor chip (Cytiva Life Sciences). An unrelated biotinylated protein was immobilized to act as a reference surface for nonspecific binding. Measurements were made with serial dilutions of WT-VEGF-C, LS-VEGF-C, and C125S in Hepes buffer saline-P+ buffer (GE Healthcare) using single cycle kinetics. The VEGFR-2 and VEGFR-3 surfaces were regenerated using the reagents provided in the CAPture kit according to the manufacturer's instructions. All data was analyzed with the Biacore T100 evaluation software 2.0 with a 1:1 Langmuir binding model.

### Murine VEGF-C Library Construction and Selection

Crystal structures of VEGF-C/VEGFR-2 complex (PDB ID 2XIW) and VEGF-C/VEGFR-3 (PDB ID 4BSK) were aligned to analyze VEGF-C and its receptor interface. Fourteen positions in m VEGF-C which were in contact with both mVEGFR-2 and m VEGFR-3 were identified to create mutation library (Table 1). Synthesized degeneration primers covering these mutated residues were used to assemble the library (Table 2). The PCR products were further amplified with primers containing homology to the vector and co-electroporated into EBY100 competent yeast together with linearized pCT-GCN42 vector. The resulting library was later measured to contain 10⁸ transformants. FIG. 3 depicts VEGF-C mutein library design. Structure guided primer libraries were designed for generation of ˜10³ VEGF-C mutein combinations. Structures of VEGF-C binding to VEGFR-3 and VEGFR-2 were studied to identify key residues on the surface of binding sites. Amino acids that can have significant polarization changes in these residues were chosen for designing of primers.

TABLE 1
VEGF-C library design.

| | VEGF-C library design | | | | |
|---|---|---|---|---|---|
| | | | | SEQ ID | |
| | Residue | Codon | AA | NO: | #AA |
| Site1 | 112T | RST | STAG | 27 | 4 |
| (IgC2 Domain Chain N) | 115L | VDA | LQRIKRVEG | 28 | 9 |
| | 119D | VDT | DGVINSLHR | 29 | 9 |
| | 126Q | VAW | QHNKDE | 30 | 6 |
| Site2 | 144T | VYT | TLPIVA | 31 | 6 |
| (IgC3 Domain Chain M) | 145N | RNA | NITSVADG | 32 | 8 |
| | 149K | VVA | KRTPQAEG | 33 | 9 |
| Site1 | 163N | VRM | NHDQKERSG | 34 | 12 |
| (IgC2 Domain Chain N) | 164S | VRM | NHDQKERSG | 34 | 12 |
| | 165E | VVA | EAGTKRPQ | 35 | 9 |
| | 1841 | VYT | ILVPTA | 36 | 6 |
| | 186V | VYT | VILPTA | 37 | 6 |
| | 188L | NWT | FLIVYHND | 38 | 8 |
| | 192P | VVA | PQRTKRAEG | 39 | 9 |

| | |
|---|---|
| Theoretical diversity | 3E+12 |

TABLE 2
VEGF-C library primers

| | | SEQ | | | |
|---|---|---|---|---|---|
| | | ID | | | Primer |
| # | Primer sequence | NO: | Length | Tm | style |
| 1 | GCGGTTCTCTGGAAGTTCTGTTCCAGGGTCCGGGATCC | 40 | 38 | 72 | regular |
| 5 | GGaCTGCAGTGCATGAACACCAGCACAGGTTACCTCA | 41 | 54 | 73 | degene |
| | GCAAGACGTTGTTTGAA | | | | rated |
| 7 | AAACCAGTCACAATCAGTTTTGCCAATCACACTTCCTG | 42 | 48 | 72 | codon |
| | CCGGTGCATG | | | | |
| 8 | cagatgaTCTGTAAACATCCAGTTTAGACATGCACCGGCA | 43 | 48 | 70 | |
| | GGAAGTGT | | | | |
| 9 | GGATGTTTACAGAtcatctggatctagcGGATCTGCGGCCGCTT | 44 | 46 | 72 | |
| | CT | | | | |
| 10 | TCGGAGATAAGCTTTTGTTCGCCACCAGAAGCGGCCG | 45 | 44 | 74 | |
| | CAGATCC | | | | |
| 2 | CCTCACGTGGCATGCAWTBAGTCTTTCTCCACTCATTA | 46 | 79 | 74 | |
| | HBAATACTTTTTHBGATCTCASYGGATCCCGGACCCTG | | | | |
| | GAA | | | | |
| 3 | TGCATGCCACGTGAGGTGTGTATAGATGTGGGGAAGG | 47 | 85 | 77 | |
| | AGTTTGGAGCAGCCVYTRNAACCTTCTTTVVACCTCCA | | | | |
| | TGTGTGTCCG | | | | |
| 4 | TGGTGTTCATGCACTGCAGtCCTBBKYBKYBGCAGCAA | 48 | 70 | 78 | |
| | CCtCCACATCTGTAGACGGACACACATGGAGG | | | | |
| 6 | AAAACTGATTGTGACTGGTTTTBBGCCTTGTGAAWNA | 49 | 68 | 71 | |
| | GGARBTGTARBTTCAAACAACGTCTTGCTGA | | | | |

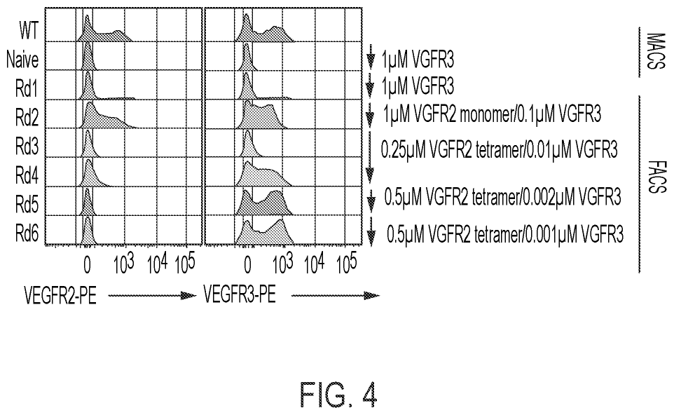

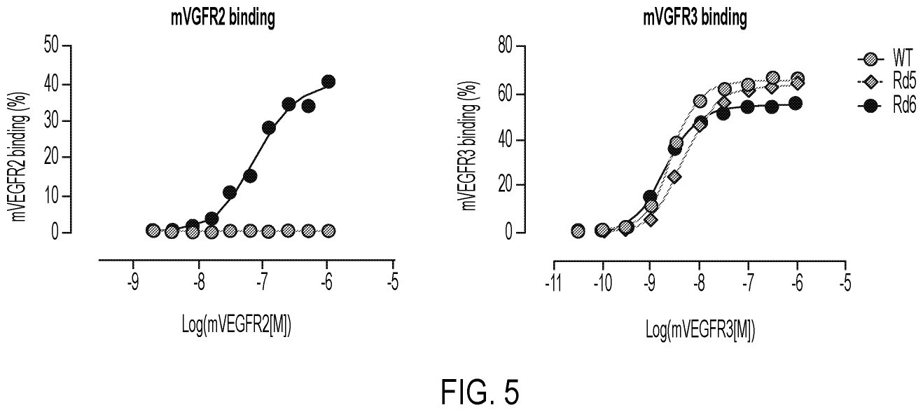

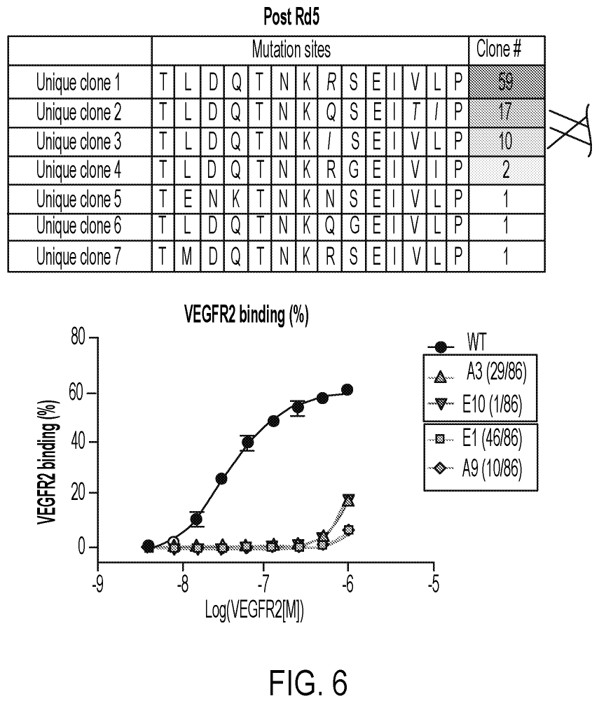

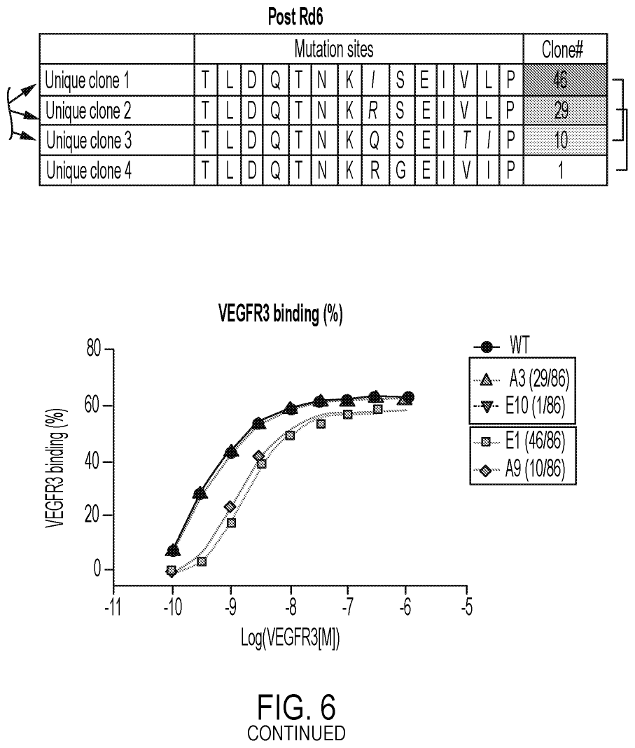

Transformed yeasts were recovered and expanded in SDCAA medium at 30° C. and induced in SGCAA medium at 20° C. for 24-48 h. Naïve libraries were selected with 2 rounds of 1 μM mVEGFR-3 to enrich mVEGFR-3 positive binders using LS column (Miltenyi, #130-042-401) and magnetic selection. Starting from round 3, yeast populations were counter-selected with 1 μM m VEGFR-2 monomer and selected with 100 nM m VEGFR-3 using flow cytometer sorting with Sony SH800 cell sorter. For rounds 4-6, counter-selection reagent m VEGFR-2 concentration was gradually increased to 0.25 μM (round 4) and 0.5 M (round 5 & 6) tetramer, while m VEGFR-3 concentration was decreased to 10 nM (round 4), 2 nM (round 5), or 1 nM (round 6). Yeasts post each round of selection were kept and boosted simultaneously to check their binding affinity towards VEGFR-2 and VEGFR-3 when all selections were completed. After the final round of selection, VEGF-C plasmids were extracted from expanded SDCAA cultured yeasts and transformed into E. coli for colony sequencing (Table 3). Converged unique clones were re-transformed into yeast to titrate their binding preference to VEGFR-2 and VEGFR-3. The final selected VEGF-C mutants were expressed by insect cell pFastbac expression system and purified in FPLC with SEC columns. Mono disperse proteins were finally cleared with endotoxin removing for in vivo animal treatment. As shown in FIG. 4, wild type VEGF-C showed binding towards yeasts expressing both VEGFR-2 and VEGFR-3 (top row, WT). First two rounds selected for binding towards VEGFR-3. In subsequent rounds, decreasing amounts of VEGFR-3 were utilized to select for high binding affinity muteins towards VEGFR-3. At the same time, VEGFR-2 was included and negatively selected against in order to enrich for muteins that lost binding affinity towards VEGFR-2 with high binding to VEGFR-3. FIG. 5 depicts confirmation of receptor affinity preference post final rounds of selection. Muteins that were selected for positively for VEGFR-3 and negatively against VEGFR-2 display high binding for VEGFR-3 and no binding towards VEGFR-2 even at concentrations of the receptor 100× higher than wild type binding. FIG. 6 depicts confirmation of receptor affinity preference post final rounds of selection. Clonal muteins after round 5 and 6 of selection were sequenced to identify unique clones that were enriched for in the mutein population. Unique mutein residues were identified to create specific mutations into wild type VEGF-C for validation.

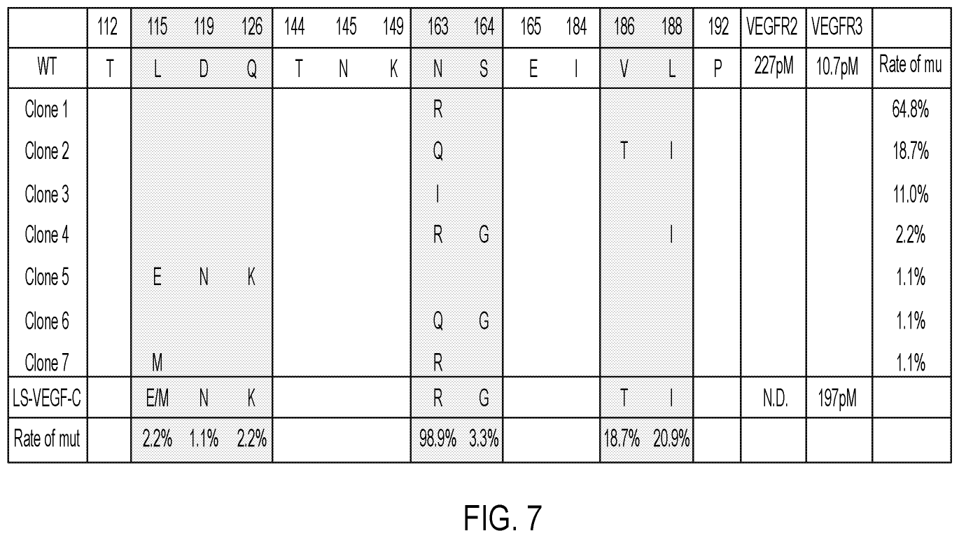

FIG. 7 depicts confirmation of receptor affinity preference post final rounds of selection. Clonal muteins after round 5 and 6 of selection were sequenced to identify unique clones that were enriched for in the mutein population. Unique mutein residues were identified to create specific mutations into wild type VEGF-C for validation

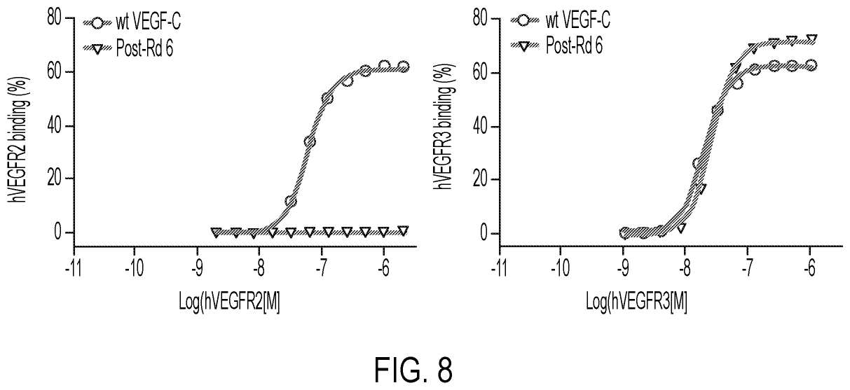

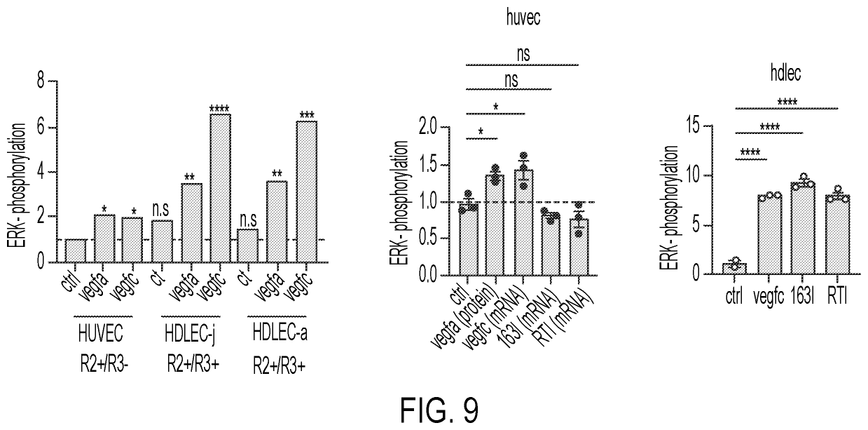

FIG. 8 depicts confirmation of receptor affinity preference post final rounds of selection against human VEGF-receptors. Muteins that were selected for positively for VEGFR-3 and negatively against VEGFR-2 display high binding for VEGFR-3 and no binding towards VEGFR-2 even at concentrations of the receptor 100× higher than wild type binding.

TABLE 3
VEGF-C final round sequencing results.

| | Mutation sites | | | | | | | | | | | | | | Clone # |
|---|---|---|---|---|---|---|---|---|---|---|---|---|---|---|---|
| Unique clone 1 | T | L | D | Q | T | N | K | I | S | E | I | V | L | P | 46 |
| Unique clone 2 | T | L | D | Q | T | N | K | R | S | E | I | V | L | P | 29 |
| Unique clone 3 | T | L | D | Q | T | N | K | Q | S | E | I | T | I | P | 10 |
| Unique clone 4 | T | L | D | Q | T | N | K | R | G | E | I | V | I | P | 1 |

### Engineering Lymphatic-Specific VEGF-C (LS-VEGF-C)

Increased lymphatic drainage demonstrates therapeutic benefit for pathologies such as lymphedema, in which the build-up of fluid due to destroyed lymphatic infrastructure is directly addressed². With a more immunological perspective, leveraging meningeal lymphatics, increased brain-antigen drainage by lymphangiogenesis and facilitated enhanced immunological response against glioblastoma³. These applications not only demonstrate the therapeutic potential of harnessing lymphangiogenesis, but also its versatility of application even in immune-privileged spaces.

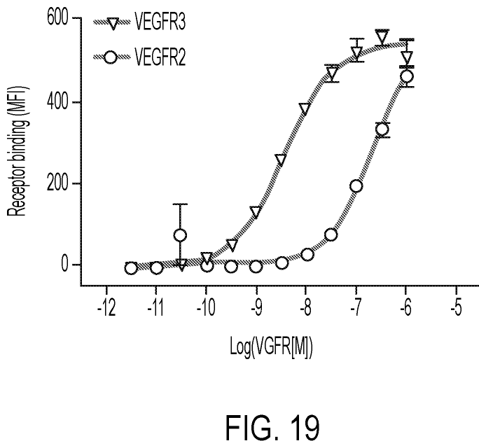

Previous studies show that VEGFR-2 primarily facilitates angiogenesis⁴ while VEGFR-3 facilitates lymphangiogenesis⁵⁻⁸. Vascular endothelial growth factor C (VEGF-C) is the main growth factor able to promote lymphatic growth and pump, however its pleiotropism, binding to both VEGFR-2 and VEGFR-3 (FIG. 19), has limited its use as a therapy and as a molecular tool to study in vivo signaling. Furthermore, VEGF-C's ability to promote sufficient lymphangiogenesis is variable, often requiring high doses for significant effect⁹; altogether, these shortcomings prevent full realization of the therapeutic potential in VEGF-C-driven lymphangiogenesis.

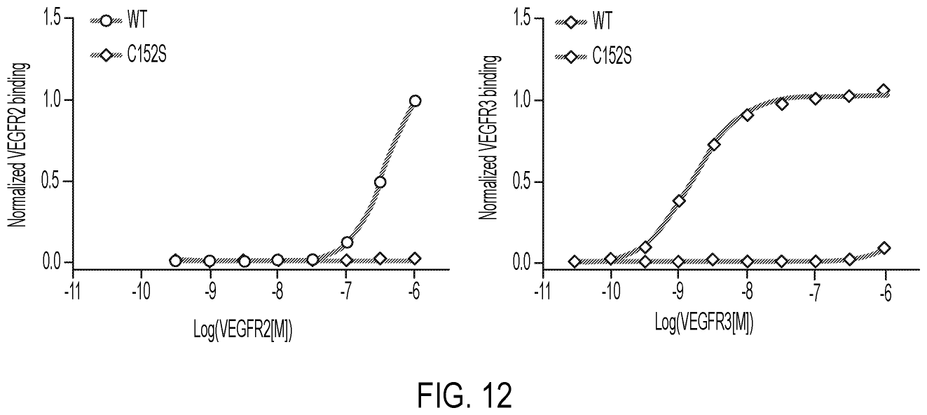

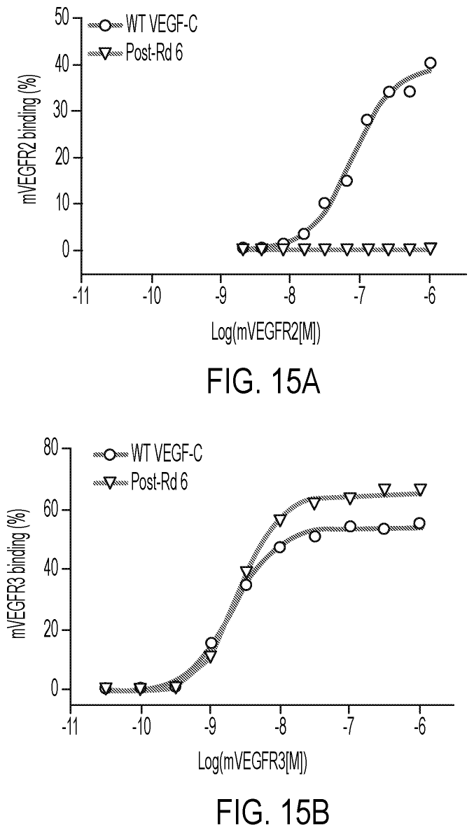

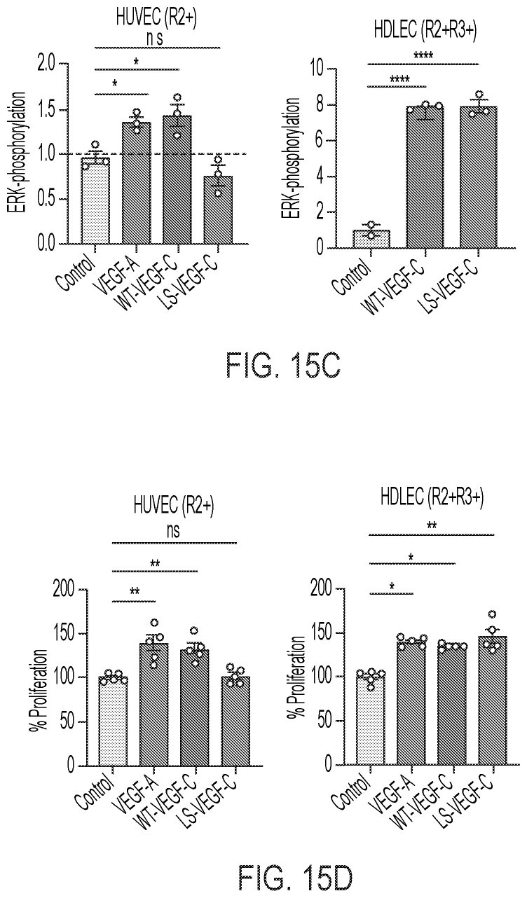

Towards overcoming these limitations, it was sought to engineer a more selective, yet potent VEGF-C mutein. To develop a mutant VEGF-C that would only bind to VEGFR-3 and prompt lymphangiogenesis, 14 residues at the receptor binding interface were identified by structural analysis for randomization (FIG. 3). Using a yeast-display directed evolution platform (FIG. 2B), a library was created which underwent six rounds of selection for selectivity to VEGFR-3 and counter-selection against VEGFR-2 (FIG. 4). After this final selection, a population of variants showed conserved, exclusive binding to VEGFR-3, while showing no binding to VEGFR-2 for both human (FIG. 8) and mouse versions of the receptor (FIGS. 15A and 15B). Of note, VEGFR-3 binding of these variants outperform those of a previously established VEGFR-3 specific ligand VEGF-C152S/C-156S (FIG. 12). Inspection of the clones identified 7 key residues 115, 119, 126, 163, 164, 186 and 188, resulting in our final variant (LS-VEGF-C); biophysical characterization by surface plasma resonance showed that LS-VEGF-C preserved picomolar affinity for VEGFR-3 with complete suppression of VEGFR-2 affinity compared to WT-VEGF-C (FIGS. 6 and 7).

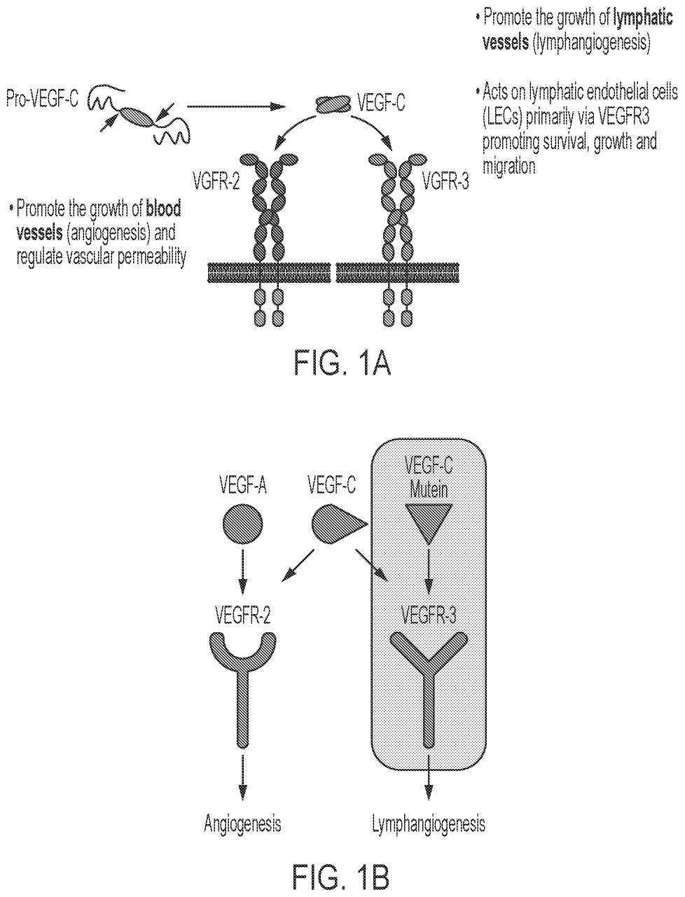

To evaluate whether functionality was conserved, HUVEC and HDLEC cells were used, which are vascular endothelial and lymphatic endothelial cells respectively. Upon administering VEGF-A, LS-VEGF-C, and WT-VEGF-C, which demonstrate binding to VEGFR-2, VEGFR-3, or both (FIG. 1B), downstream signaling was detected by measuring ERK activation in the cells. HUVECs, which have expression of VEGFR-2, demonstrated similar enhanced ERK activation by VEGF-A and WT-VEGF-C, but not LS-VEGF-C (FIG. 15C). In contrast, HDLECs demonstrated similar ERK phosphorylation between WT-VEGF-C and LS-VEGF-C (FIG. 15C), which reflects the conserved VEGFR-3 binding and signaling in the mutant protein. Investigation into cellular proliferation upon VEGF administration confirms this trend; vascular endothelial cells have increased proliferation with VEGF-A and WT-VEGF-C, while lymphatic endothelial cells have increased proliferation with any VEGF, but most statistically significantly with LS-VEGF-C (FIG. 15D). Due to the lymphatic endothelial cells displaying both VEGFR-2 and VEGFR-3, it is not wholly surprising that VEGF-A and WT-VEGF-C also leads to increased proliferation of lymphatic endothelial cells.⁵ Together, these data demonstrate that LS-VEGF-C is a new mutant form of VEGF-C highly specific for VEGFR-3 binding and lymphangiogenesis.

mRNA/Nanoparticle

VEGF-C wild type and mutein coding mRNA was synthesized by TriLink Bio Technologies with full substitution of pseudouridine and 5-methylcytosine bases, capped using CleanCap reagent AG and poly-adenylated (120A). mRNA was mixed at a ratio of 1 ug per 0.1 μL of in vivo JETPEI (Polyplus Transfection) and vortexed for 30 seconds and incubated in room temperature for 15 minutes before use.

### Cells

Human umbilical vascular endothelial cells (HUVECs) and human dermal lymphatic endothelial cells (HDLECs) were obtained from (Promocell). They were cultured in MV media (Promocell) with supplements (0.05 mL fetal calf serum/mL, 0.004 mL endothelial cell growth supplement/mL, 10 ng recombinant human EGF/mL, 90 ug heparin/mL, 1 ug hydrocortisone/mL).

### Western Blot

HEK293T cells were transfected with the VEGFC mRNA constructs combined with lipofectamine. Supernatant was taken from these cells and HUVEC, HDLEC-j, or HDLEC-a cells were incubated with it. In other experiments, VEGF-C or VEGF-A proteins were directly added to media and put on top of cells. Samples were lysed in RIPA buffer and boiled for 5 minutes with sample buffer. In other experiments, HUVEC and HDLEC cells were treated with 100 ng/ml of VEGF-A, WT-VEGF-C, or LS-VEGF-C in RPMI medium with 1% FBS supplement. Samples were trypsinized (0.05%), quenched with RPMI medium (1% FBS supplement). Supernatant was aspirated, and cell pellets were resuspended and subsequently lysed in RIPA buffer, after which they were boiled for 5 minutes with sample buffer.

Western blotting was performed in a manner similar to that previously reported³. In short, 10% gels were used and run at 10 mA per gel for 30 min and 40 mA per gel until appropriate separation of ladder. Wet transfer was performed at 120 mA per gel for 90 min on ice. After blocking with milk-TBST and 3 washes with TBST, anti-pERK was used at a concentration of 1:1000 and incubated overnight in the cold room. After washing, HRP-conjugated anti-rabbit secondary antibodies were used at a concentration of 1:500 at room temperature for 2 h and imaged using the ChemiDoc MP imaging system (Bio-Rad).

### Mice

Six-to-ten-week-old mixed sex C57BL/6J (WT), DBA2J and DBA/2J-Gpnmb+/SjJ mice were purchased from Jackson Laboratory, and subsequently bred and housed at Yale University. All procedures used in this study (sex-matched, age-matched) complied with federal guidelines and the institutional policies of the Yale School of Medicine Animal Care and Use Committee.

### MTT Assay

Using a 96 well plate, 10,000 cells per well were seeded. Cells were first starved for 12 hours in RPMI medium without supplements, before undergoing a wash with PBS and then being treated to experimental conditions. Negative controls were treated to RPMI medium with 1% FBS supplement. Experimental conditions consisted of the addition of 100 ng/ml of VEGF-A, WT-VEGF-C, or LS-VEGF-C to the negative control media (RPMI medium with 1% FBS supplement). A standard curve was made with cell titration. 10 uL of a 12 mM MTT stock solution was then added to each sample. After homogenizing each well by pipetting up and down, absorbance was made at 570 nm and cell count was determined by matching to the standard curve.

### Isolation of Endothelial Cells

The cornea, retina, choroid and optic nerve was isolated for single cell dissociation. These tissues were digested with 1 mg ml⁻¹ collagenase D (Roche) and 30 ug ml⁻¹ DNase I (Sigma-Aldrich) in RPMI at 37° C. for 45 min. Samples were then pipetted up and down to mechanically dissociate the tissue and filtered through a 70-um filter. Samples were then spun down at 5 minutes at 500× g. Cell pellet was then resuspended in FACS buffer (PBS with 2% FBS and 1 mM EDTA) for staining.

### Flow Cytometry

Nonspecific binding was blocked using a Fc receptor-blocking solution (TruStain FcX™, 101320, BioLegend) for 10 minutes at 4° C. prior to immunostaining. Subsequently, the cells were stained with corresponding antibodies for 30 min at 4° C. Cells were then washed to remove excess antibodies and resuspended in FACS buffer. Samples were run on an Attune NxT flow cytometer and then analyzed using FlowJo software (10.8.1, Tree Star).

For AKT phosphorylation staining, surface markers were first stained on ice for 30 min. Cells were then fixed, and stained following the directions of the BD Phosflow kit. Samples were run on an Attune NxT flow cytometer and then analyzed using FlowJo software (10.8.1, Tree Star).

### Measurements of Intraocular Pressure

Intraocular pressures were measured following the consensus recommendations (https://iovs.arvojournals.org/article.aspx?articleid=2778419). Mice were exposed to brief exposure of isoflurane for sedation, after which intraocular pressure was measured by an iCare tonometer. IOP measurement is an average of 6 measurements in one sedation session. Mice were positioned consistently, such that the probe was perpendicular to the surface of the eye when the measurements were taken.

Administrations into the Eye

After intraperitoneal injection of a mixture of ketamine (50 mg kg⁻¹) and xylazine (5 mg kg⁻¹), mice then received topical, intravitreal, or intracameral administration. Topical administration involved placing 5 uL of 1 ug/uL solutions onto the eye. Intravitreal and intracameral administration involved a small puncture along the edge of the cornea to allow access for a Hamilton syringe. For intracameral administration, the needle travels into the anterior chamber. For intravitreal administration, the needle enters the vitreous humor space. Both intravitreal and intracameral administration involved 1 ug/uL solutions. After administration, the eyes were then covered with an artificial tear ointment. Any eyes not being evaluated was also covered with an artificial tear ointment to avoid drying out.

### Combinatorial Therapy

After IVT administration of LS-VEGF-C, mice were then anesthetized with an intraperitoneal injection of ketamine (25 mg kg⁻¹) and xylazine (2.5 mg kg⁻¹) mixture two days later. Subsequently, eyes received topical administration of an FDA-approved drug to assess for combinatorial effect.

### Microbead Model

Adapted from Sappington et al.¹⁸, the mice were anesthetized through intraperitoneal injection of ketamine (50 mg kg⁻¹) and xylazine (5 mg kg⁻¹) and then received intracameral administration of polysterene beads. Artificial tear ointment was then placed topically to avoid drying out the cornea.

### Intravitreal AAV Injections and Imaging

WT mice were intravitreally injected with AAVs (dose) with PBS, VEGF-A (concentration), WT-VEGF-C (concentration), or LS-VEGF-C (concentration). After 1 day, the mice were anesthetized intraperitoneally injecting a mixture of ketamine (50 mg kg⁻¹) and xylazine (5 mg kg⁻¹). 100 uL of dye at a concentration of 20 mg/mL was also injected intraperitoneally. Eyes were dilated with 1% tropicamide. After 5 minutes incubation, the mice were placed upon a mount for fundus, fluorescein angiography, OCT imaging on Phoenix Micron IV.

### Absorbance Measurement of Evans Blue

For Evans blue readouts, instead of the dye, Evans blue was injected intraperitoneally. After 4 hours, the mouse was then euthanized and perfused with PBS. The eyes were then isolated and homogenized with beads, before running on a plate reader (Abs_max at 620 nm).

### Tissue Processing and Microscopy

For retinal wholemounts, mice were first enucleated, and the eyes were then fixed in 1% formaldehyde. Upon careful removal of the optic nerve, cornea, and the sclera, the isolated retina was then dissected into quarters with cuts halfway to the optic nerve After staining with Brn3a and DAPI, confocal imaging was done on a LeicaSP8 microscope.

For cross-section of the optic nerve, the optic nerve was fixed in 4% before being processed and embedded in resin/OTC/paraffin mixtures. Blocks were then sectioned on an microtome/cryostat. Cross-sections were then visualized on a transmission electron microscope.

### NMDA Excitation Study

Adapted from Schlüter et al.³¹, 10 nmol of NMDA was delivered intravitreally and RGCs were evaluated at day 1 post injection by confocal microscopy of retinal wholemounts, as described above.

### Statistical Analysis

All statistical analysis was performed using GraphPad Prism software. Data were analyzed with a two-tailed unpaired Student's t-test or paired Student's t-test with Prism software. Statistical significance is defined as *P<0.05, **P<0.01, and ***P<0.001.

### Intravitreal Injection

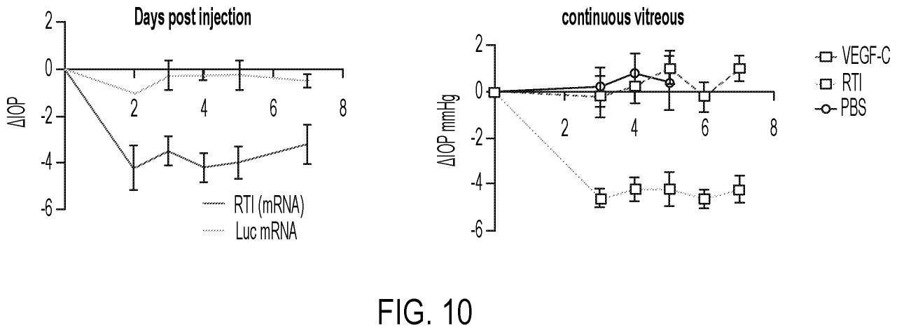

Mice were anaesthetized using ketamine and xylazine. One drop of 0.5% Tropicamide was applied to the eyes. Mouse was positioned to expose the superior nasal region of the eye and using a 33 g needle, the superior nasal sclera at the level of the pars plana was punctured. Mouse head was secured, and the needle was positioned at a 45 degree angle. Once the tip was inserted, 2 μL of mRNA-nanoparticle formulation was injected and needle was left in for 5 seconds to prevent backflow. Antibacterial ophthalmic ointment was applied afterwards to prevent infection and mice were placed in a heated cage until full recovery. FIG. 10 depicts the use of VEGF-C mutein in vivo. Single administration of VEGF-C muteins intravitreally resulted in sustained decrease in intraocular pressure (IOP) in a wild type mouse compared with its counterpart.

### Tumor Inoculation

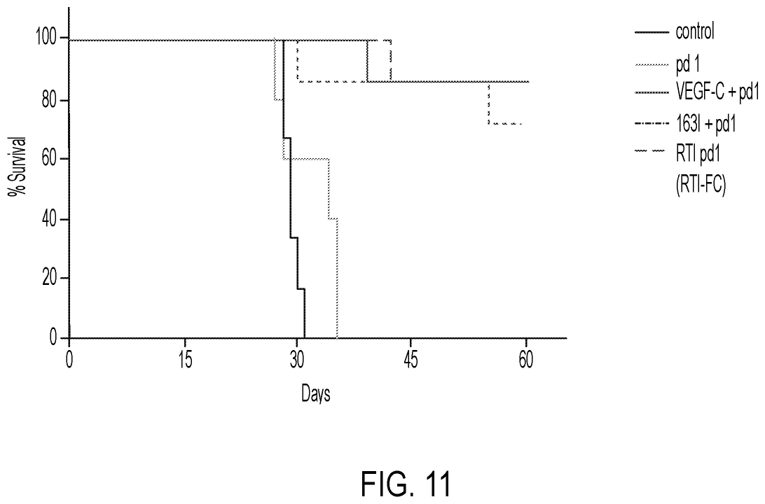

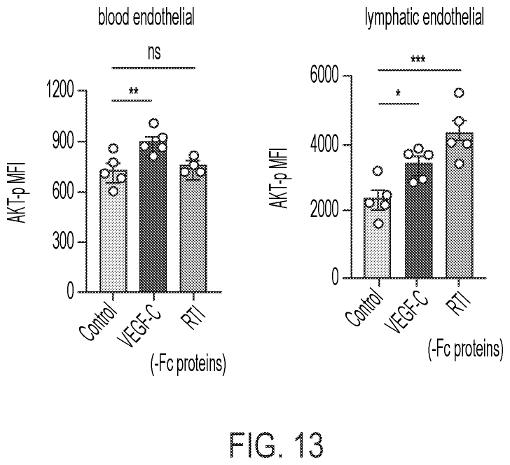

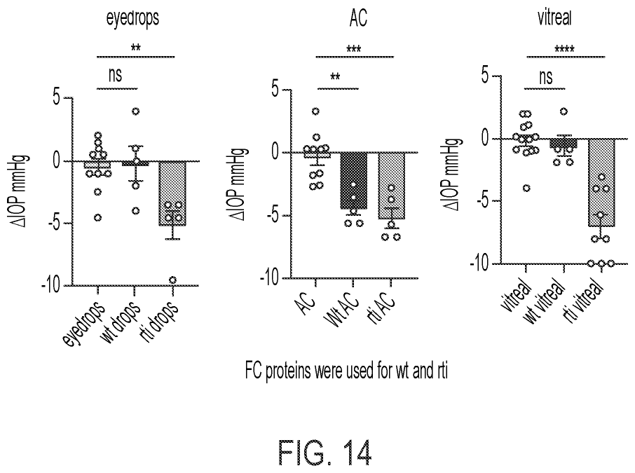

Mice were anaesthetized using a mixture of ketamine (50 mg kg⁻¹) and xylazine (5 mg kg⁻¹), injected intraperitoneally. Mice heads were shaved and then placed in a stereotaxic frame. After sterilization of the scalp with alcohol and betadine, a midline scalp incision was made to expose the coronal and sagittal sutures, and a burr hole was drilled 2 mm lateral to the sagittal suture and 0.5 mm posterior to the bregma. A 10-μl Hamilton syringe loaded with tumor cells was inserted into the burr hole at a depth of 2.5 mm from the surface of the brain and left to equilibrate for 1 minute before infusion. A micro-infusion pump (World Precision Instruments) was used to infuse 3 μl of tumor cells at 1 μl min⁻¹. Once the infusion was finished, the syringe was left in place for another minute before removal of the syringe. Bone wax was used to fill the burr hole and the skin was stapled and cleaned. Following intramuscular administration of analgesic (meloxicam and buprenorphine, 1 mg kg⁻¹), mice were placed in a heated cage until full recovery. FIG. 11 depicts the use of VEGF-C mutein in vivo. Wild type VEGF-C and VEGF-C muteins were evaluated in vivo for the treatment of brain tumors. In combination with anti-PD-1 antibodies, VEGF-C muteins showed significant therapeutic benefits treating brain tumors. Furthermore, as shown in FIG. 12, reported VEGF-C mutants in literature loses binding affinity towards both VEGFR-2 and VEGFR-3. Mutants such as C152S, which has been previously reported in literature, loses activity towards both VEGFR-2 and VEGFR-3, losing its potent lymphangiogenic activity. As shown in FIG. 13, isolated VEGF-C muteins of the present disclosure displayed specific signaling through VEGFR-3 in vivo. Wild type VEGF-C and VEGF-C mutein (RTI) signaling was evaluated in vivo. These vectors were injected into eyes of mice. 24 hours later, eyes were enucleated and made into single cell suspensions to check for AKT-phosphorylation. Wild type VEGF-C retained signaling through VEGFR-2 in blood endothelial cells while mutant VEGF-C(RTI) no longer had signaling. Both the wild type protein and RTI mutein signaled through VEGFR-3 in lymphatic endothelial cells. Furthermore, as shown in FIG. 14, routes of administration resulted in differential drops in IOP in vivo. VEGF-C or VEGF-C mutein (RTI) was administered either by eye drops, injection into the anterior chamber (AC) or intravitreally. While in the AC, both wild type and the mutein had similar activity, the mutein showed selective ability to decrease eye pressure when administered via eyedrops or intravitreally.

### Intracisterna-Magna Injection

For intracisterna-magna injections, mice were anaesthetized using ketamine and xylazine, and the dorsal neck was shaved and cleaned with alcohol. A 2-cm incision was made at the base of the skull, and the dorsal neck muscles were separated using forceps. After visualization of the cisterna magna, a Hamilton syringe with a 15-degree, 33-gauge needle was used to puncture the dura. Three microliters of mRNA vector (4-5 ug) was administered per mouse at a rate of 1 μl min⁻¹. After completion of the injection, the needle was left in to prevent backflow for an additional 3 minutes. The skin was stapled and cleaned and the same postoperative procedures were performed as for the tumor inoculations.

### Example 2. LS-VEGF-C Demonstrates Potent Therapeutic Efficacy in Multiple Settings

To confirm that LS-VEGF-C acts through VEGFR-3 signaling in vivo, LS-VEGF-C and WT-VEGF-C were first administered into wild-type mice and then endothelial cells (CD45⁻CD31⁺) were evaluated for activation of downstream signaling using phos-flow. WT-VEGF-C showed significant increase of AKT-phosphorylation in endothelial cells regardless of VEGFR-3 expression (FIG. 13). In comparison, LS-VEGF-C induced significantly greater AKT-phosphorylation in cells that expressed VEGFR-3 (CD45⁻CD31⁺VEGFR3⁺) while not in cells that did not express VEGFR-3 (CD45⁻CD31⁺VEGFR3⁻), suggesting that the in vivo activity of LS-VEGF-C was reflective of what was observed in vitro.

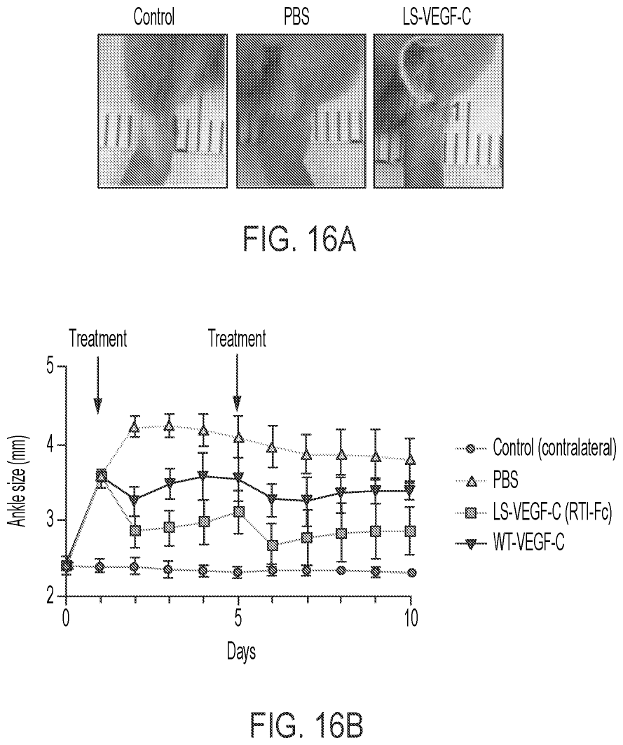

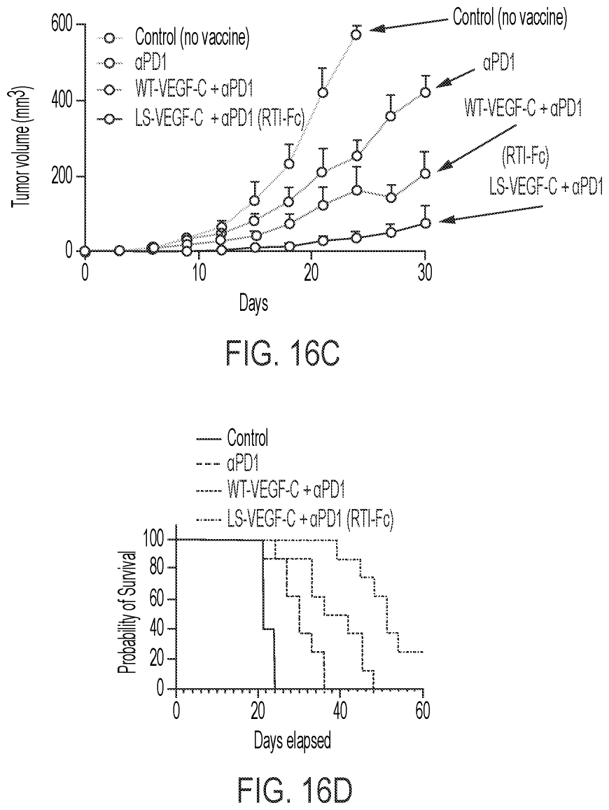

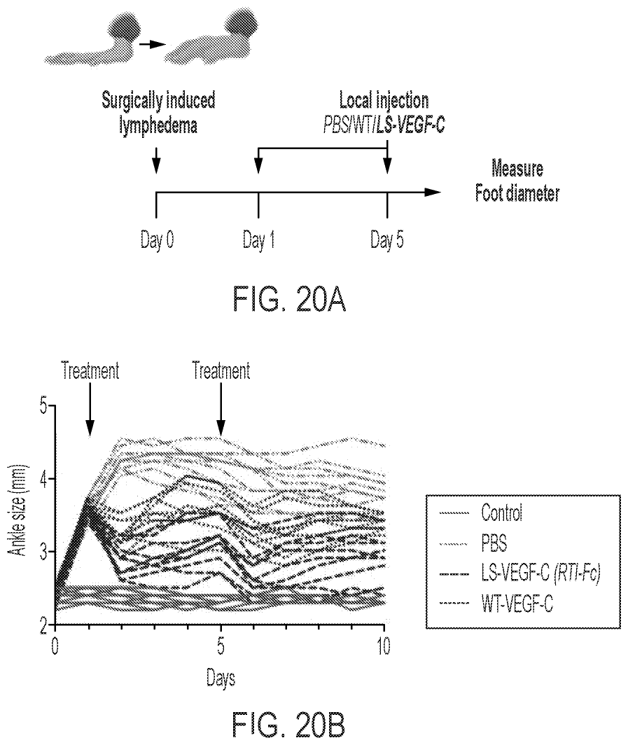

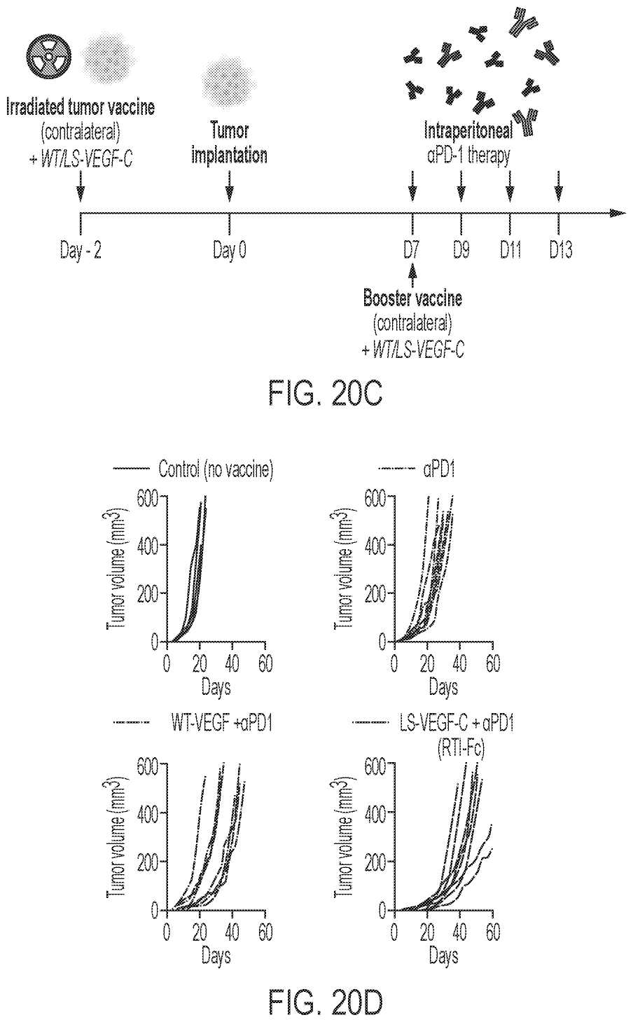

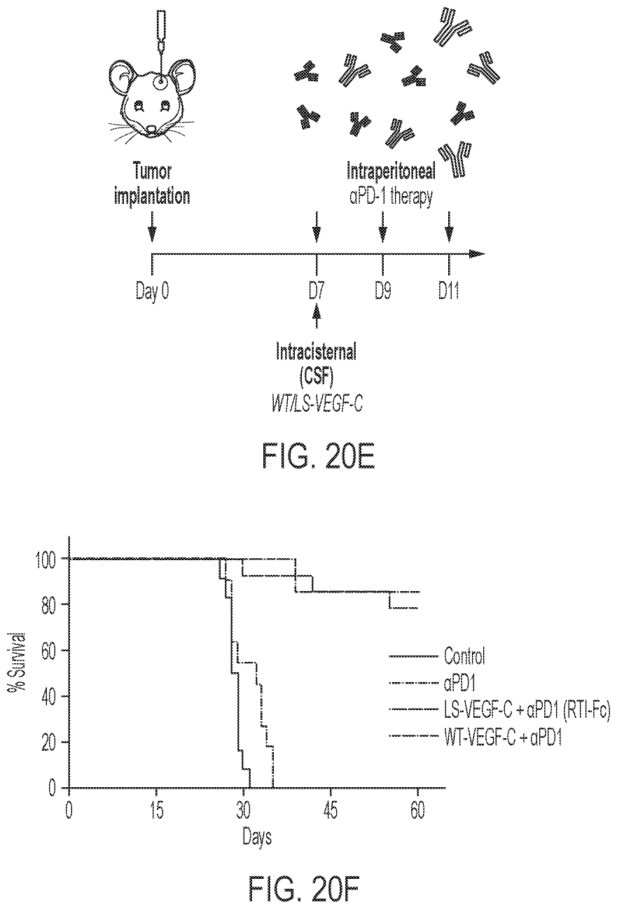

Since its discovery, VEGF-C's potential of lymphatic modulation was demonstrated in vivo in many pathological processes. LS-VEGF-C's ability to provide therapeutic lymphangiogenesis was tested herein in several of these models. First, a mouse model of hind limb lymphedema¹⁰ was induced through local lymphatic ablation by surgical ablation. Administration of LS-VEGF-C was able to resolve swelling more effectively than WT-VEGF-C and to an extent comparable to normal (FIGS. 16A, 16B, 20A and 20B). Not only was it effective as a single agent in this setting after a single dose, the molecule showed continued activity after multiple administrations, highlighting its potential to be a therapeutic for chronic diseases. Second, LS-VEGF-C was evaluated as an adjuvant for anti-PD-1 cancer immunotherapy in a mouse model of melanoma. In both tumor volume reduction and survival, LS-VEGF-C demonstrated superior preclinical efficacy, compared to WT-VEGF-C (FIGS. 16C, 16D, 20C and 20D). Finally, LS-VEGF-C was utilized as an adjuvant therapy to checkpoint inhibitor therapy for the treatment of glioblastoma. LS-VEGF-C demonstrated activity comparable to the WT-VEGF-C, resulting in survival of most of the animals treated, in an otherwise fatal outcome (FIGS. 20E and 20F). These set of experiments establish the conservation of LS-VEGF-C's biological functions in both controlling biophysical properties and immunological outcomes while demonstrating its superiority over the wild type growth factor as an in vivo pharmacologic agent.

### Example 3. Harnessing Ocular Lymphatics for Biophysical Modulation

Previous studies show that the eye has a lymphatic system draining the anterior compartment; lymph vessels in the conjunctiva¹¹ and ciliary body¹² have previously been described, as well as a posterior glymphatic clearance system.¹³ It was recently identified by our team that ocular lymphatics are compartmentalized and established anterior and posterior part of the eye have distinct lymphatic drainage systems. By expanding these lymphatic structures through lymphangiogenesis, it was hypothesized herein that ocular administration of LS-VEGF-C would lead to increased drainage and drop intraocular pressure in the eye.

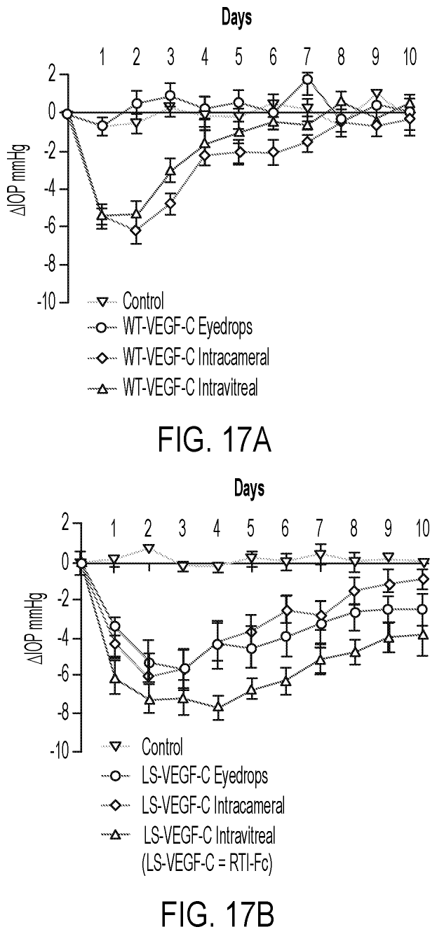

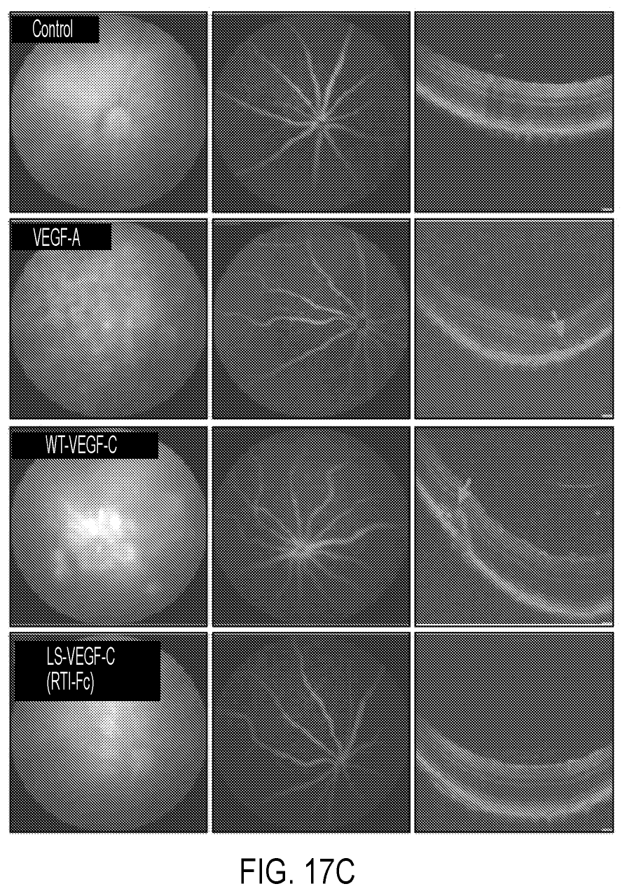

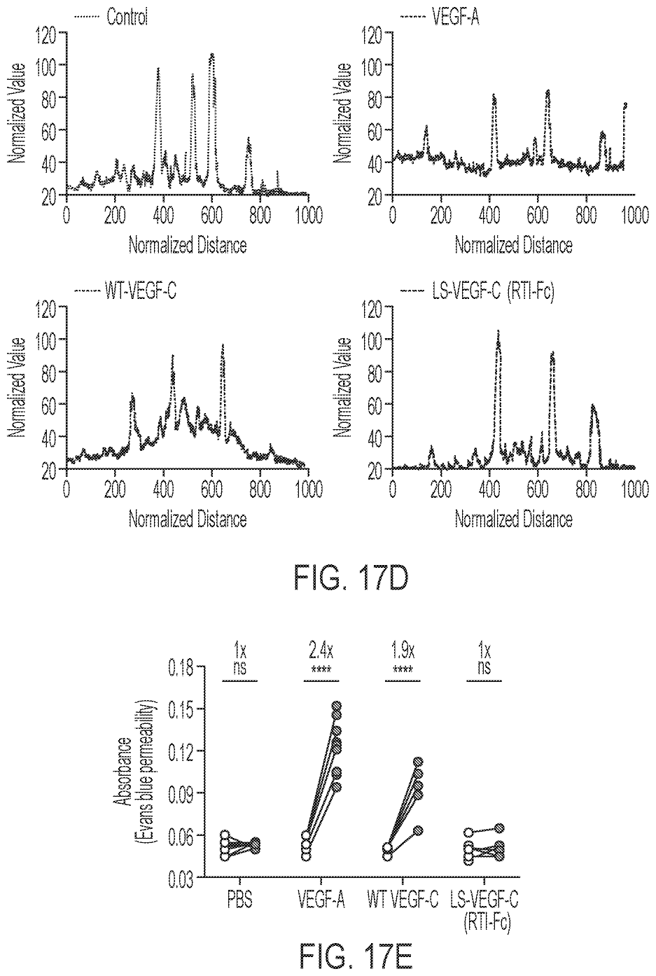

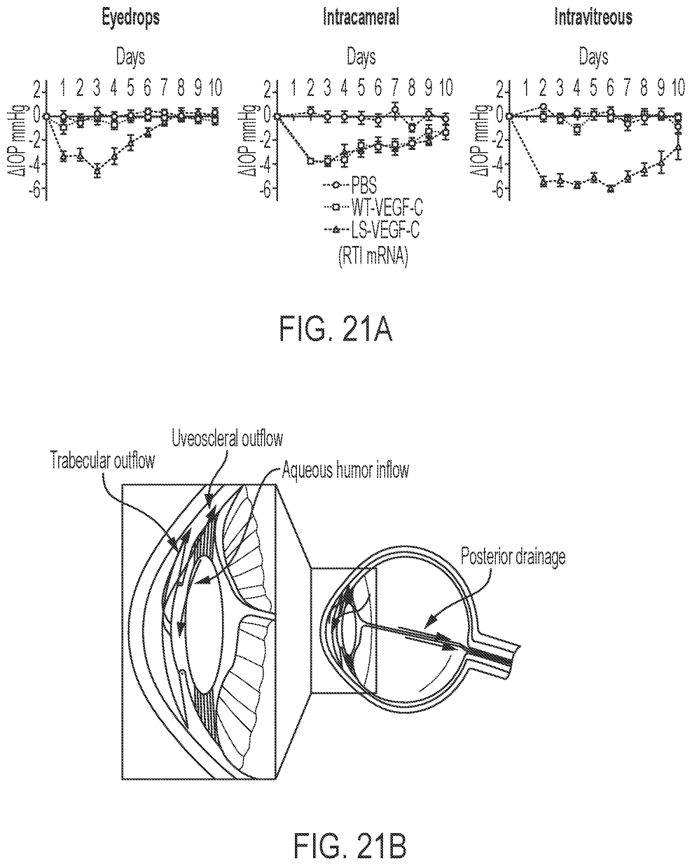

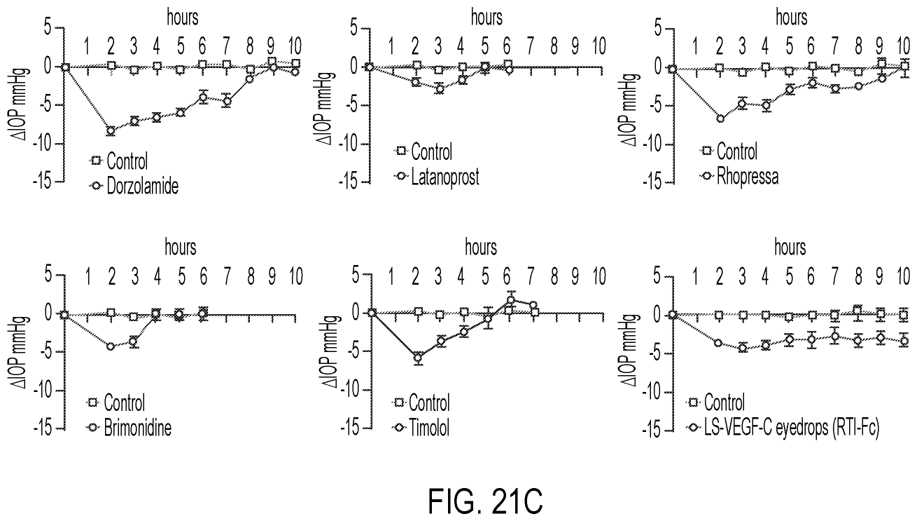

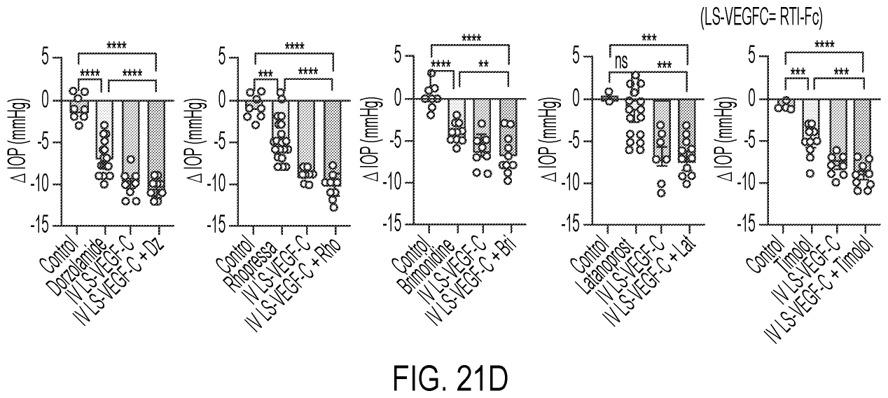

WT-VEGF-C lead to lower intraocular pressure (IOP) measurements by intracameral injection, similar to previous reports¹⁴, and after intravitreal injection (FIG. 17A). In comparison, LS-VEGF-C led to sustained and enhanced reduction in IOP after any administration method, whether topical, intracameral, or intravitreal (FIG. 17B). The greater impact and versatility of LS-VEGF-C may stem from VEGFR-3 specificity, with WT-VEGF-C suffering from having two possible receptors to bind to, i.e., VEGFR-2 acting as a VEGF-C-sink¹⁵. This may also contribute to the lack of a phenotypic effect with WT-VEGF-C by eyedrop, as well as the fast clearance and multiple ocular barriers to overcome which hinder many topical applications¹⁶. Interestingly, in a mRNA form, WT-VEGF-C fails to effect a reduction in IOP when administered topically or intravitreally, but performs similarly as the more efficacious LS-VEGF-C when administered intracamerally (FIG. 21A, highlighting the importance of both the delivery method and vehicle. Importantly, LS-VEGF-C demonstrated a prolonged effect in dropping eye pressure, with drops in IOP observable within a few hours, and persisting up to days in comparison to commonly used glaucoma eye drops that showed few hours of activity (FIGS. 21B and 21C). LS-VEGF-C's activity as a single agent was significantly stronger than the other glaucoma drugs and combinatorial therapy was not required to achieve a plateau in IOP decrease (FIG. 21D). The potency of LS-VEGF-C in a normotensive IOP model demonstrated the translation of our in vitro findings to a physiological phenomenon.

### Example 4. LS-VEGF-C Disengages Vascular Side-Effects In Vivo

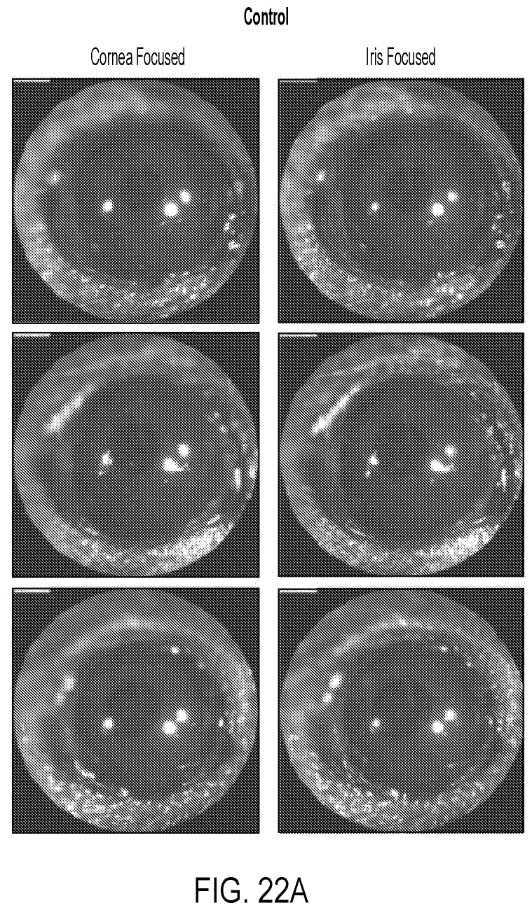

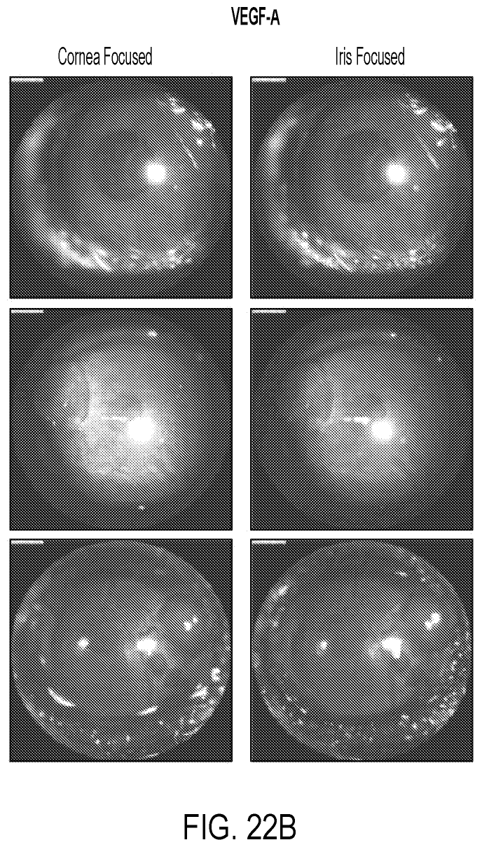

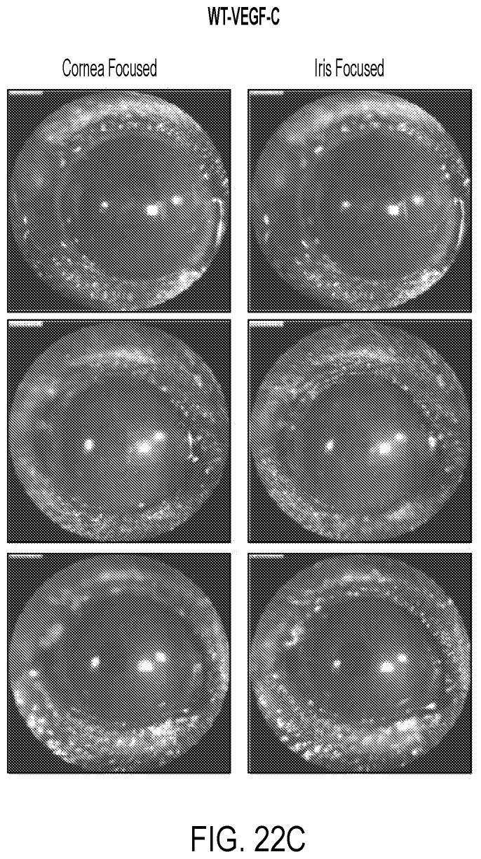

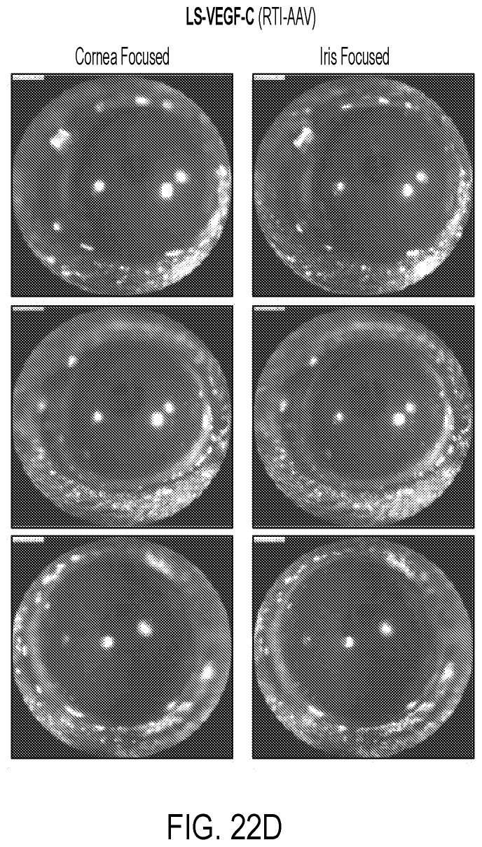

To evaluate the off-target effects and vascular changes from VEGF growth factors, retinal vasculature was imaged after intravitreal injection of VEGF-A, WT-VEGF-C, and LS-VEGF-C. Looking at fundus photos, compared to the PBS control, VEGF-A and WT-VEGF-C demonstrated spots of hyper-reflection along with increased vascular tortuosity (FIG. 17C left column). Fluorescein angiography demonstrated similar anatomical vascular findings with enhanced visibility of permeability into the retinal parenchyma. VEGF-A and WT-VEGF-C resulted in increased dye presence in the extravascular space (FIG. 17C middle column) which could be quantified by measuring fluorescence intensity across the image (FIG. 17D). In control and LS-VEGF-C administered retinas, no intensity above 40 was observed except overlaying major blood vessels, however in VEGF-A and WT-VEGF-C administered retinas large regional patches displayed intensity above 40. Optical coherence tomography (OCT) displayed consistent findings as the angiography, with pockets of fluid in near the choroid/retinal pigment epithelial layers after VEGF-A or WT-VEGF-C administration in the eye but not with LS-VEGF-C administration (FIG. 17C right column). As a parallel method of validating these findings, Evans blue absorbance was measured after systemic injection of Evans blue and perfusion of mice with PBS to evaluate for retinal vascular permeability. Similar to the angiography data, these data revealed that LS-VEGF-C was able to mitigate increased vascular permeability, displaying unchanged, healthy retinal vasculature (FIG. 17E). In the anterior side of the eye, VEGF-A resulted in uncontrolled angiogenesis in the iris and cornea along with corneal cloudiness, indicative of edema or fluid leakage into the anterior chamber (FIG. 22B). WT-VEGF-C resulted in a similar cloudiness of the cornea but without obvious neovascularization, while LS-VEGF-C displayed physical findings similar to the control (FIGS. 22C and 22D). Not only does LS-VEGF-C demonstrate utility by showing mitigation of unwanted vascularity, its properties as a novel molecular tool elucidate the contrasting in vivo effects of VEGFR-2 and VEGFR-3 signaling.

### Example 5. LS-VEGF-C Provides Neuroprotection in Ocular Hypertension

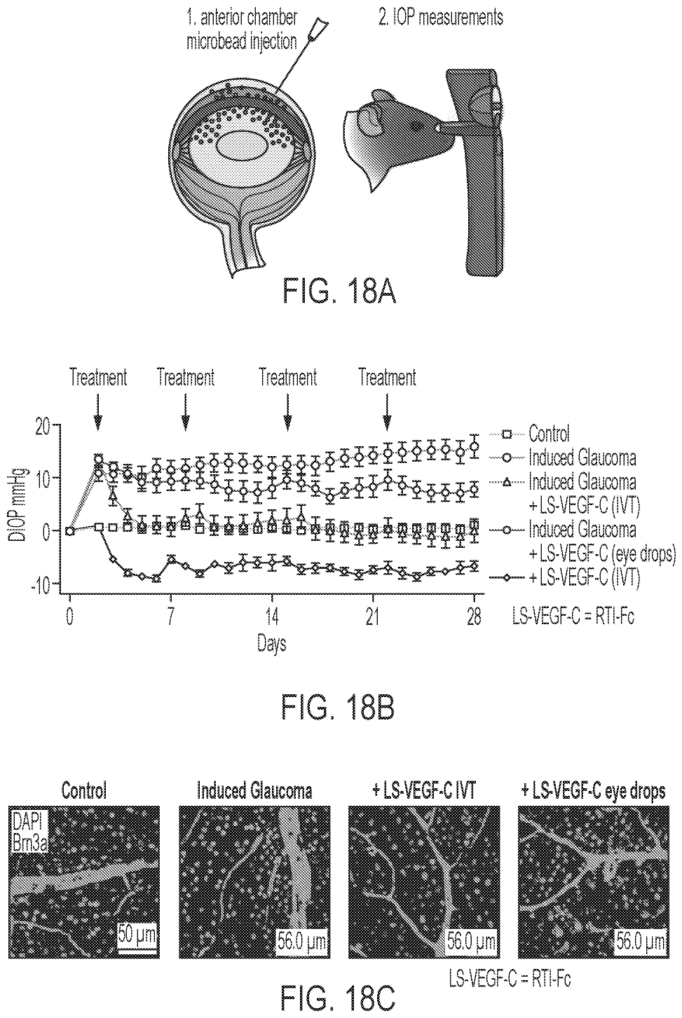

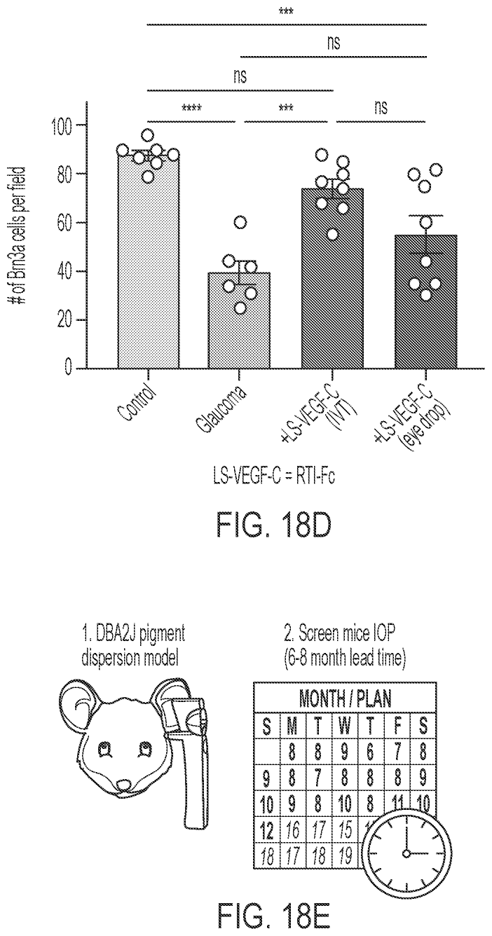

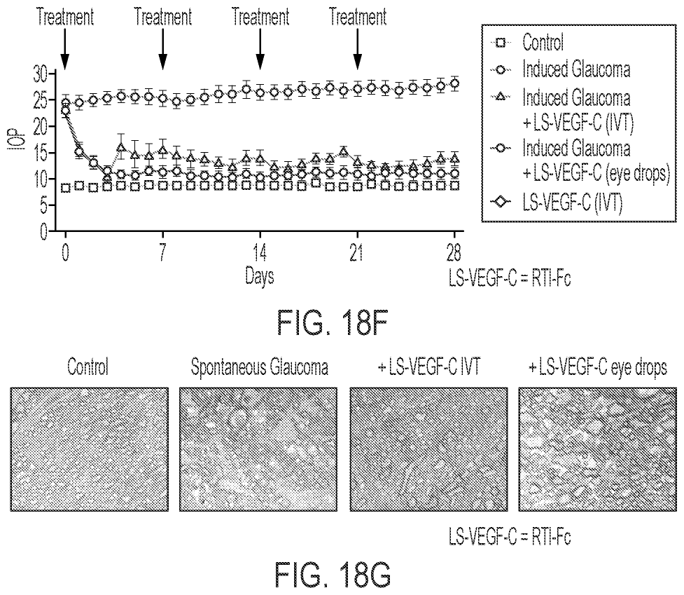

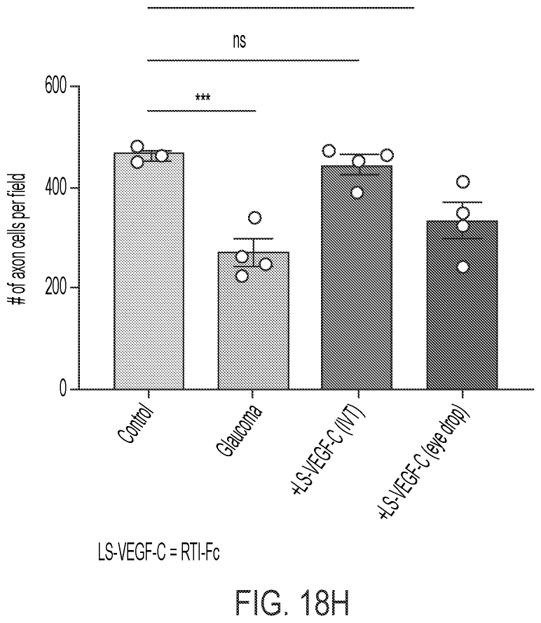

Having demonstrated IOP decreasing efficacy in wild-type mice, it was examined whether LS-VEGF-C could offer therapeutic benefits in glaucoma models. Glaucoma is characterized as a neurodegenerative disease, due to retinal ganglion cell (RGC) death, and is often associated with elevated intraocular pressure¹⁷. A microbead model was first used, which blocks outflow pathways in the eye and induces elevated IOP in a short time period¹⁸, reminiscent of acute closed angle glaucoma (FIG. 18A). Mice demonstrated an average of 20 mmHg IOP increase after the bead administration into the anterior chamber. LS-VEGF-C was administered in two different routes to evaluate its efficacy. Intravitreal (IVT) administration of LS-VEGF-C was able to revert the elevated IOP to baseline on average. Upon closer look, it was observed that this effect was bimodal, with mice having IOP decreases of 10 mmHg, values similar to wild type mice treated with LS-VEGF-C, or mice not having great responses and having persistently elevated IOP. Notably, topically administered LS-VEGF-C offered limited benefits. Mice saw decreases in eye pressure but it was not sustained and never below their baseline measurements (FIG. 18B). Imaging of RGCs, indicated by Brn3a staining, demonstrated that LS-VEGF-C lends neuroprotection to RGCs. A clear reduction of RGCs was evident in the induced glaucoma condition, which was improved upon intravitreal administration of LS-VEGF-C (FIG. 18C). Although as a group, eyedrop administration of eyedrop seemed to not have a statistically significant effect, a bimodal effect was observed with retinas that had similar number of Brn3a cells as the control and others similar to the glaucomatous eye (FIG. 18D).

To further evaluate the effects of LS-VEGF-C in glaucoma, a spontaneous glaucoma mouse was utilized, the DBA2J pigment dispersion model.¹⁹ As a negative control to establish baseline, a D2-Gpnmb+ model was used, which has a normally functioning Gpnmb gene and therefore no iris pigment dispersion that leads to spontaneous glaucoma. Although the mechanical injury to the anterior chamber is similar to those of the microbead injection, this model presents a more chronic disease state with a lead time of 6-8 months to demonstrate significantly elevated IOP (FIG. 18E). After screening for mice with elevated IOP, either PBS, or LS-VEGF-C were administered topically or intravitreally. In contrast to the microbead model, both methods of administration led to IOP decreases in the range of the control mice (FIG. 18F). The RGC axons were evaluated in the optic nerve of these mice, which showed that LS-VEGF-C managed to preserve axon integrity in this glaucoma model. Interestingly, while IVT administration showed significant pressure drop and corresponding health of optic nerve axons, topical administration did not demonstrate significant preservation of axon integrity despite having similar IOP drops (FIGS. 18G and 18H).

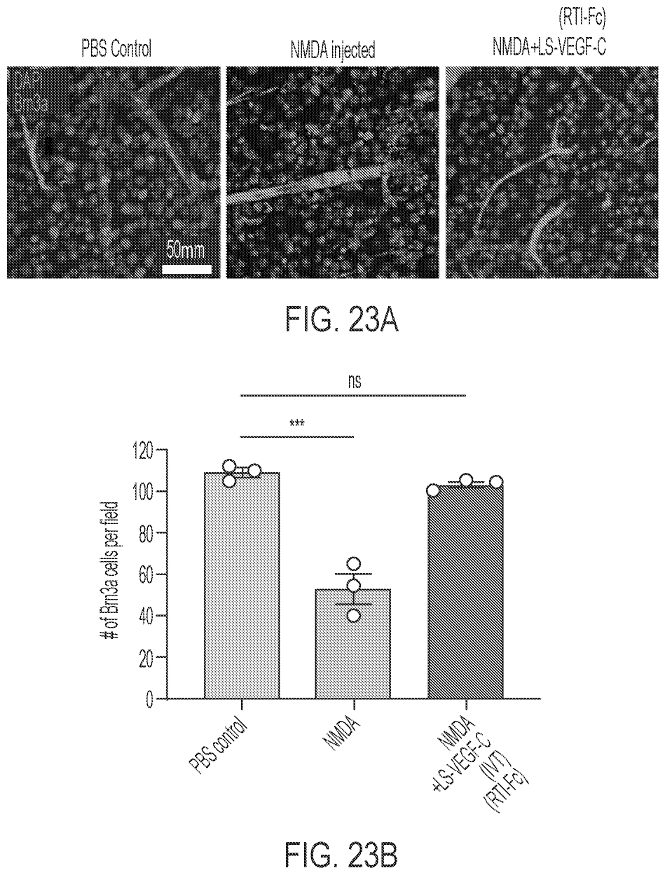

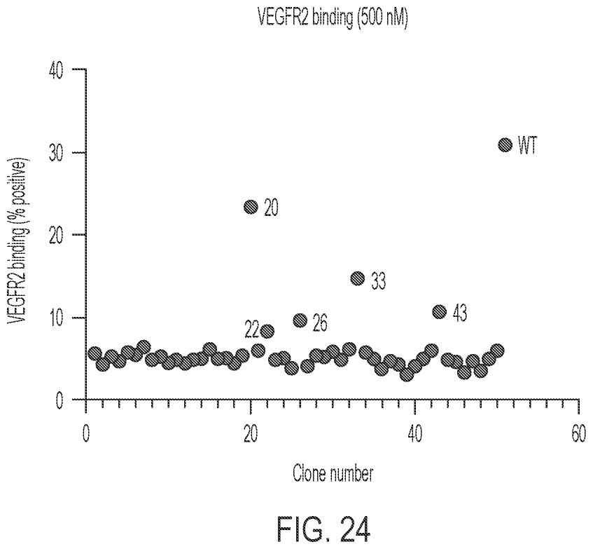

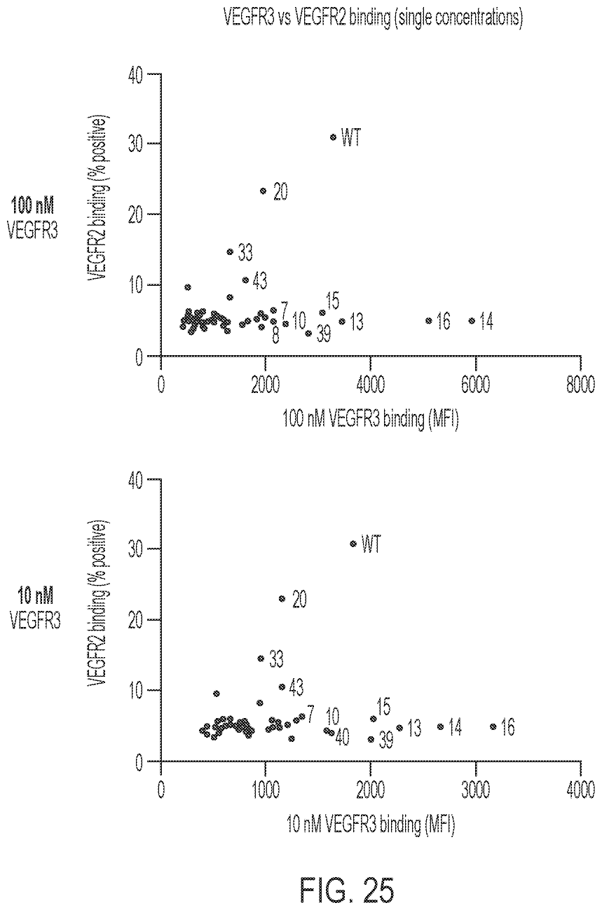

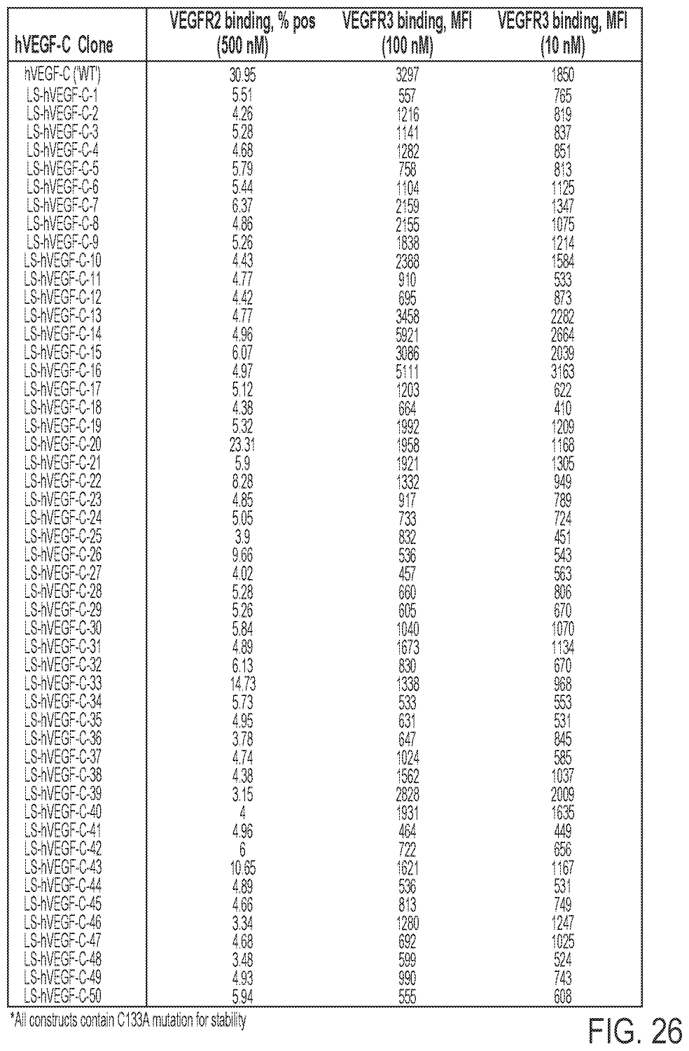

Although the exact mechanism of neuronal death in ocular-neurodegenerative diseases are still unknown, a unifying theory includes the build-up of metabolites or excitatory neuronal signals that are neurotoxic^{20,21}. Given that VEGF-C is implicated in lymphatic drainage, which allows for clearance of metabolites and macromolecules in organs^{22,23}, it was theorized herein that LS-VEGF-C may have neuroprotective properties in glaucoma beyond its ability to decrease IOP. Intravitreal injection of NMDA resulted in widespread RGC death, which was prevented with co-administration of LS-VEGF-C intravitreally (FIGS. 23A and 23B). These results collectively demonstrate that LS-VEGF-C is able to provide neuroprotection in several models of glaucoma through multi-modal mechanisms targeting IOP and clearance of neurotoxins.

### Example 6. Human LS-VEGF-C Screening

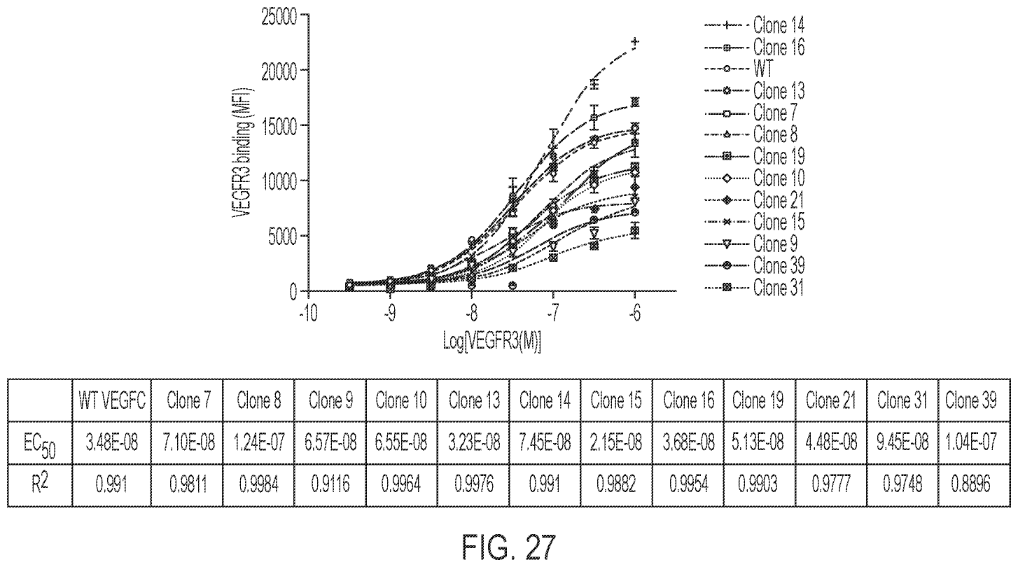

Yeast display of LS-hVEGF-C variants: Candidate human LS-VEGF-C sequences (SEQ IDs: 1-50 and 100) and WT hVEGF-C were synthesized by Twist Biosciences and inserted into a yeast display system as described above. All clones contained a C137A mutation for enhanced biochemical stability (independent of VEGFR3 bias). After induction of LS-hVEGF-C expression, yeast strains were stained with 500 nM biotinylated hVEGFR2, 100 nM biotinylated hVEGFR3, or 10 nM VEGFR3 for one hour at 4° C. Yeast were then washed with FACS buffer (PBS+0.5% BSA+0.5 mM EDTA) and stained with a fluorescent streptavidin secondary. Receptor binding was then quantified by flow cytometry. For the binding titration studies in FIG. 27, yeast were incubated with a range of hVEGFR3 concentrations: 1.00 μM, 316 nM, 100 nM, 31.6 nM, 10.0 nM, 3.16 nM, 1.00 nM, and 316 pM. Detection of hVEGFR3 binding was as described above. Table 4 shows the human clones tested. Table 5 shows the selected human clones.

TABLE 4
Human VEGF-C clones tested.

| | | mouse # | | | | | | | | | | | | | |
|---|---|---|---|---|---|---|---|---|---|---|---|---|---|---|---|
| | | 112 | 115 | 119 | 126 | 144 | 145 | 149 | 163 | 164 | 165 | 184 | 186 | 188 | 192 |
| Human | | human # | | | | | | | | | | | | | |
| clone | | 116 | 119 | 123 | 130 | 148 | 149 | 153 | 167 | 168 | 169 | 188 | 190 | 192 | 196 |
| C137A | C133A (WT) | T | L | D | Q | T | N | K | N | S | E | I | V | L | P |
| (WT) | | | | | | | | | | | | | | | |
| 1 | R163 (murine | | | | | | | | R | | | | | | |
| | clone 1) | | | | | | | | | | | | | | |
| 2 | RG | | | | | | | | R | G | | | | | |
| 3 | RI (murine | | | | | | | | R | | | | | I | |
| | clone 8) | | | | | | | | | | | | | | |
| 4 | RGI (murine | | | | | | | | R | G | | | | I | |
| | clone 4) | | | | | | | | | | | | | | |
| 5 | “RTI” | | | | | | | | R | | | | T | I | |
| 6 | RGTI | | | | | | | | R | G | | | T | I | |
| 7 | Q163 | | | | | | | | Q | | | | | | |
| 8 | QG (murine | | | | | | | | Q | G | | | | | |
| | clone 6) | | | | | | | | | | | | | | |
| 9 | QI | | | | | | | | Q | | | | | I | |
| 10 | QGI | | | | | | | | Q | G | | | | I | |
| 11 | Clone 2 (QTI) | | | | | | | | Q | | | | T | I | |
| 12 | QGTI | | | | | | | | Q | G | | | T | I | |
| 13 | I163 (murine | | | | | | | | I | | | | | | |
| | clone 3) | | | | | | | | | | | | | | |
| 14 | IG | | | | | | | | I | G | | | | | |
| 15 | II | | | | | | | | I | | | | | I | |
| 16 | IGI | | | | | | | | I | G | | | | I | |
| 17 | ITI | | | | | | | | I | | | | T | I | |
| 18 | IGTI | | | | | | | | I | G | | | T | I | |
| 19 | G164 | | | | | | | | | G | | | | | |
| 20 | I188 | | | | | | | | | | | | | I | |
| 21 | GI | | | | | | | | | G | | | | I | |
| 22 | TI | | | | | | | | | | | | T | I | |
| 23 | GTI | | | | | | | | | G | | | T | I | |
| 24 | Murine | | E | N | K | | | | | | | | | | |
| | Clone 5 | | | | | | | | | | | | | | |
| 25 | Murine | | M | | | | | | R | | | | | | |
| | Clone 7 | | | | | | | | | | | | | | |
| 26 | E115 | | E | | | | | | | | | | | | |
| 27 | ER | | E | | | | | | R | | | | | | |
| 28 | ERG | | E | | | | | | R | G | | | | | |
| 29 | EG | | E | | | | | | | G | | | | | |
| 30 | Clone 9 | | | | | | | | H | | | | T | I | |
| 31 | H163 | | | | | | | | H | | | | | | |
| 32 | Murine Clone | | | | | | | | Q | R | | | T | I | |
| | 10 (QRTI) | | | | | | | | | | | | | | |
| 33 | R164 | | | | | | | | | R | | | | | |
| 34 | RR | | | | | | | | R | R | | | | | |
| 35 | RRI | | | | | | | | R | R | | | | I | |
| 36 | RRTI | | | | | | | | R | R | | | T | I | |
| 37 | QR | | | | | | | | Q | R | | | | | |
| 38 | QRI | | | | | | | | O | R | | | | I | |
| 39 | IR | | | | | | | | I | R | | | | | |
| 40 | IRI | | | | | | | | I | R | | | | I | |
| 41 | IRTI | | | | | | | | I | R | | | T | I | |
| 42 | HR | | | | | | | | H | R | | | | | |
| 43 | R164I | | | | | | | | | R | | | | I | |
| 44 | ERR | | E | | | | | | R | R | | | | | |
| 45 | EQ | | E | | | | | | Q | | | | | | |
| 46 | EI | | E | | | | | | I | | | | | | |
| 47 | EQR | | E | | | | | | Q | R | | | | | |
| 48 | EIR | | E | | | | | | I | R | | | | | |
| 49 | EHR | | E | | | | | | H | R | | | | | |
| 50 | ERRTI | | E | | | | | | R | R | | | T | I | |

TABLE 5
Human VEGF-C clones selected.

| | | mouse residue # | | | | | | | | | | | | | |
|---|---|---|---|---|---|---|---|---|---|---|---|---|---|---|---|
| | | 112 | 115 | 119 | 126 | 144 | 145 | 149 | 163 | 164 | 165 | 184 | 186 | 188 | 192 |
| Human | | human residue # | | | | | | | | | | | | | |
| clone # | | 116 | 119 | 123 | 130 | 148 | 149 | 153 | 167 | 168 | 169 | 188 | 190 | 192 | 196 |
| C137A | C133A (WT) | T | L | D | Q | T | N | K | N | S | E | I | V | L | P |
| (WT) | | | | | | | | | | | | | | | |
| 7 | Q167 | | | | | | | | Q | | | | | | |
| 8 | QG (murina | | | | | | | | Q | G | | | | | |
| | clone 6) | | | | | | | | | | | | | | |
| 9 | QI | | | | | | | | Q | | | | | I | |
| 10 | QGI | | | | | | | | Q | G | | | | I | |
| 13 | I167 (murina | | | | | | | | I | | | | | | |
| | clone 3) | | | | | | | | | | | | | | |
| 14 | IG | | | | | | | | I | G | | | | | |
| 15 | II | | | | | | | | I | | | | | I | |
| 16 | IGI | | | | | | | | I | G | | | | I | |
| 19 | G168 | | | | | | | | | G | | | | | |
| 21 | GI | | | | | | | | | G | | | | I | |
| 31 | H167 | | | | | | | | H | | | | | | |
| 39 | IR | | | | | | | | I | R | | | | | |

### Example 7. Human LS-VEGF-C-Fc Characterization

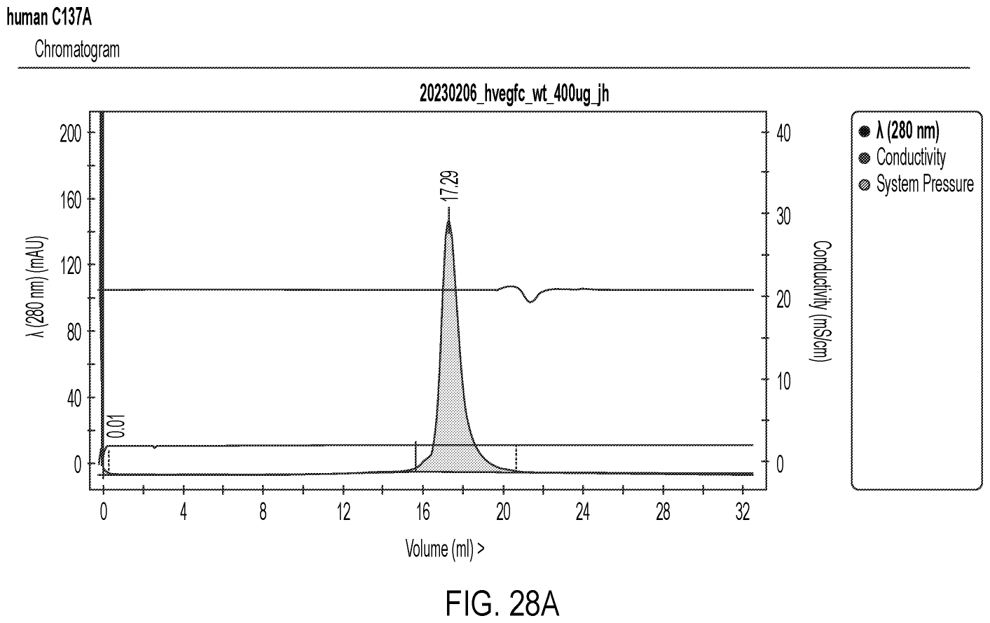

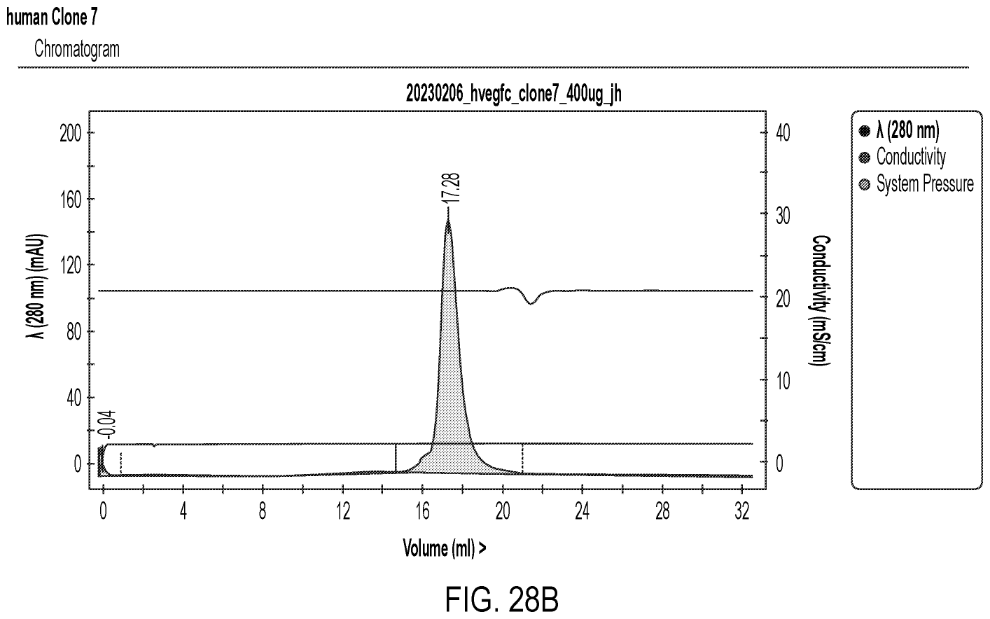

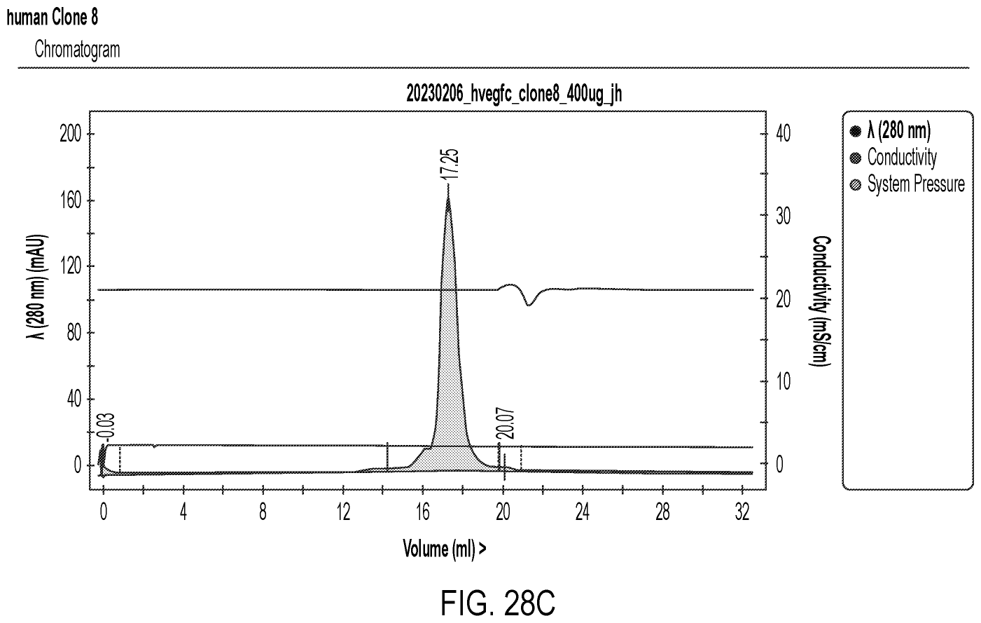

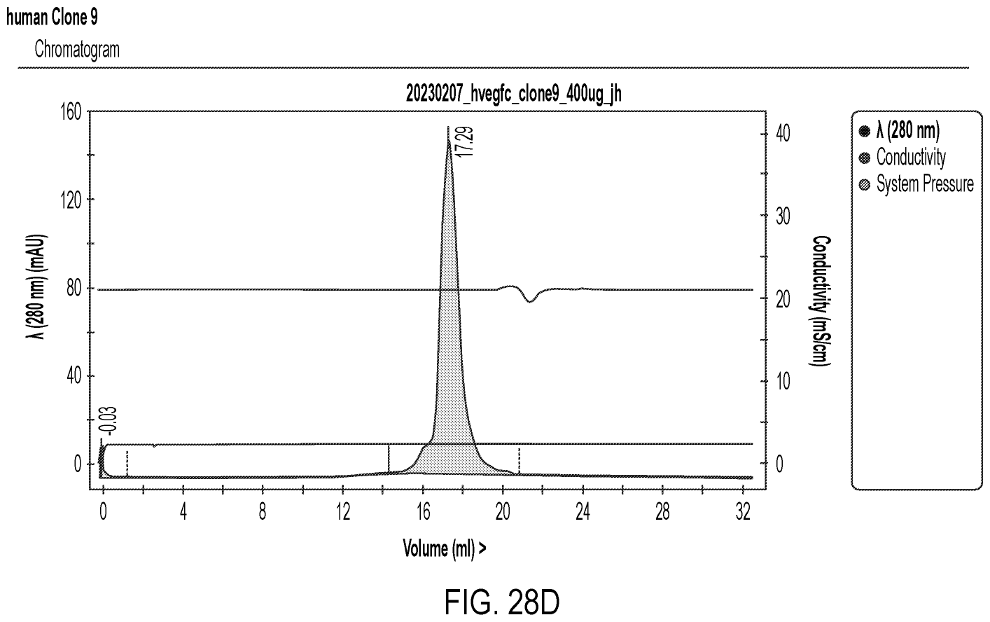

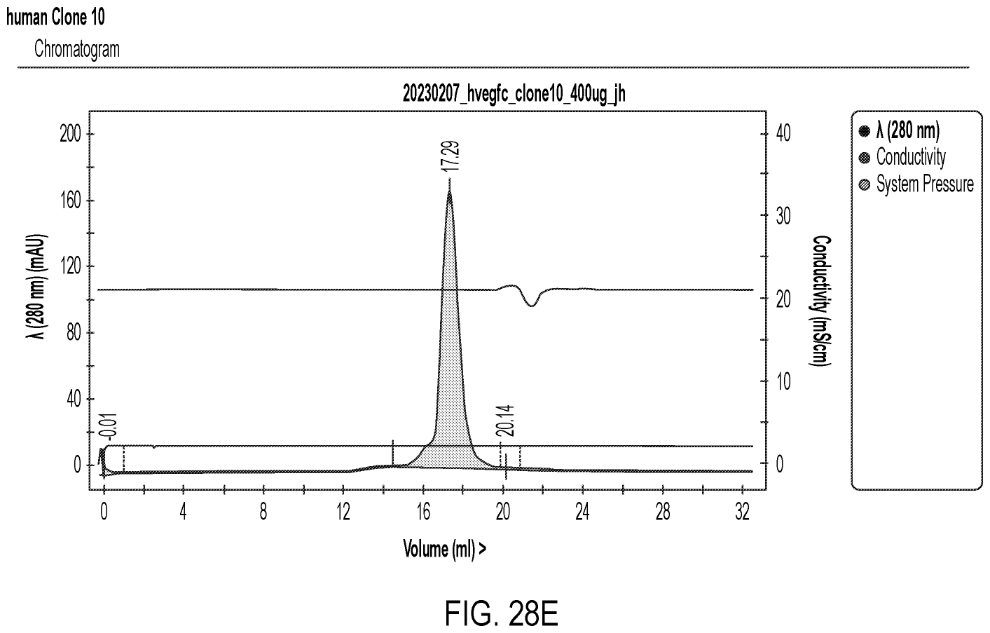

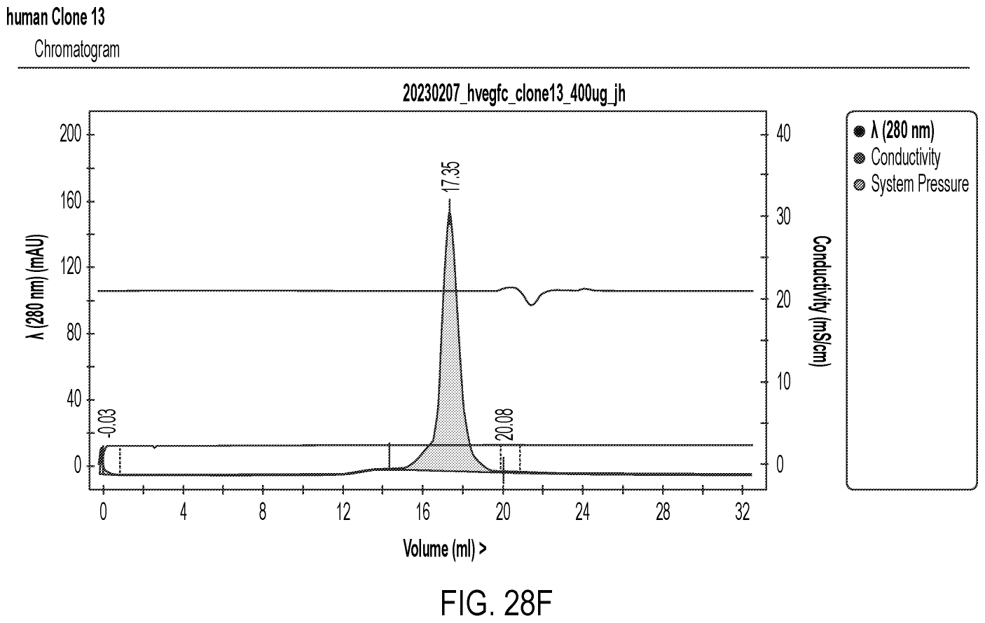

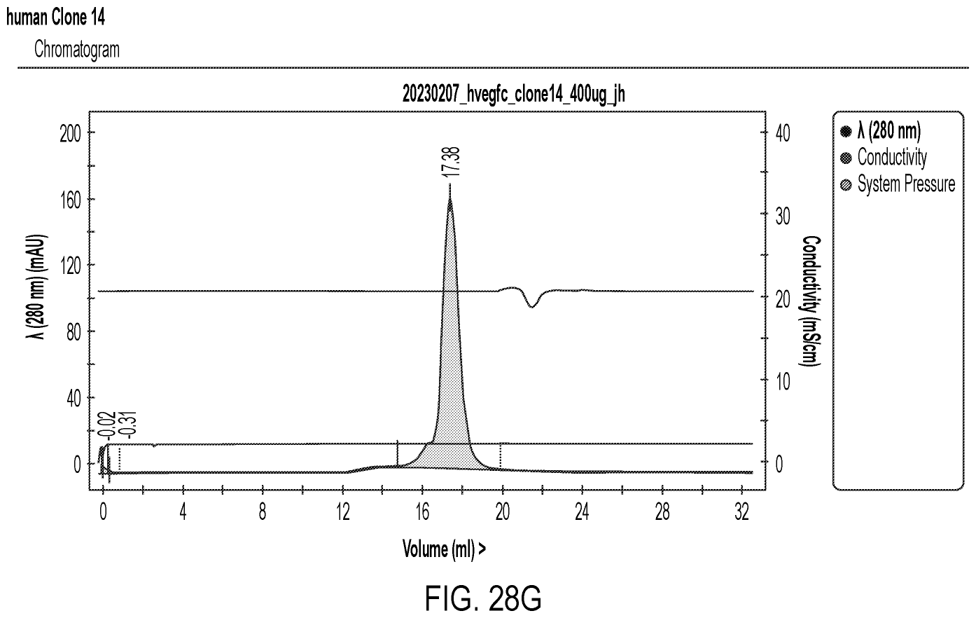

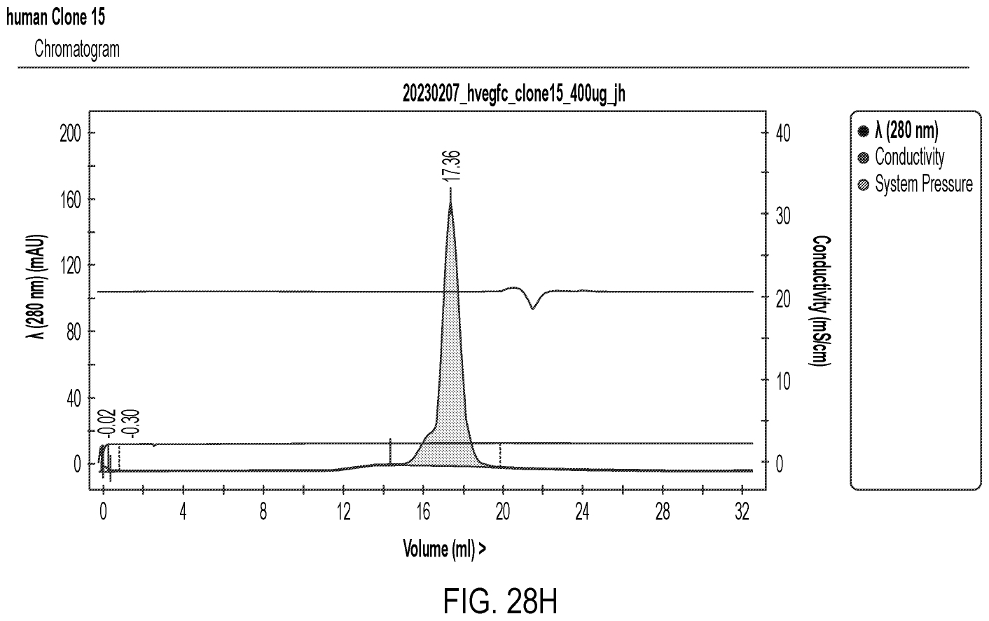

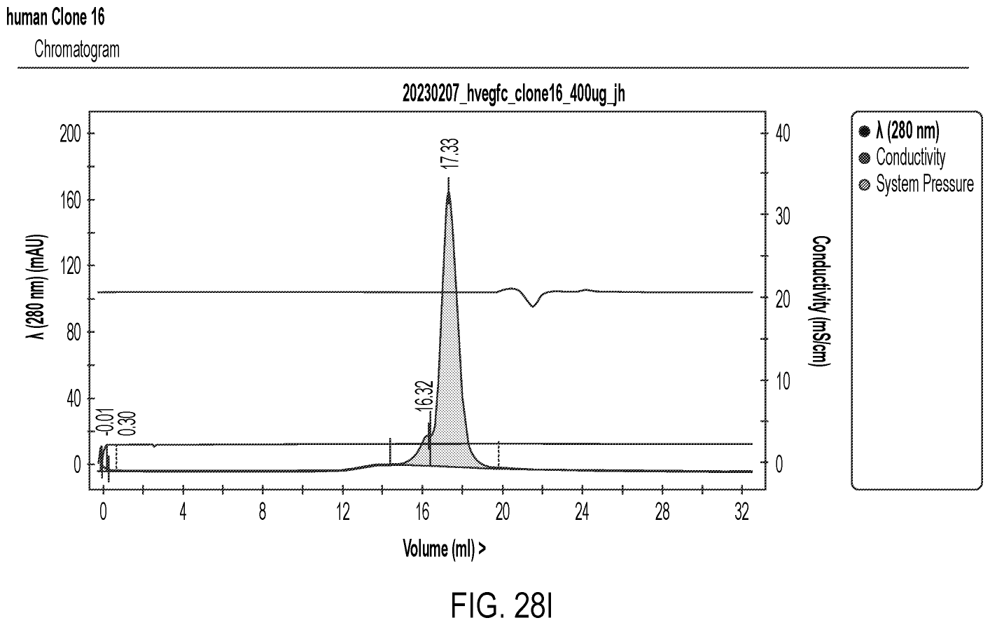

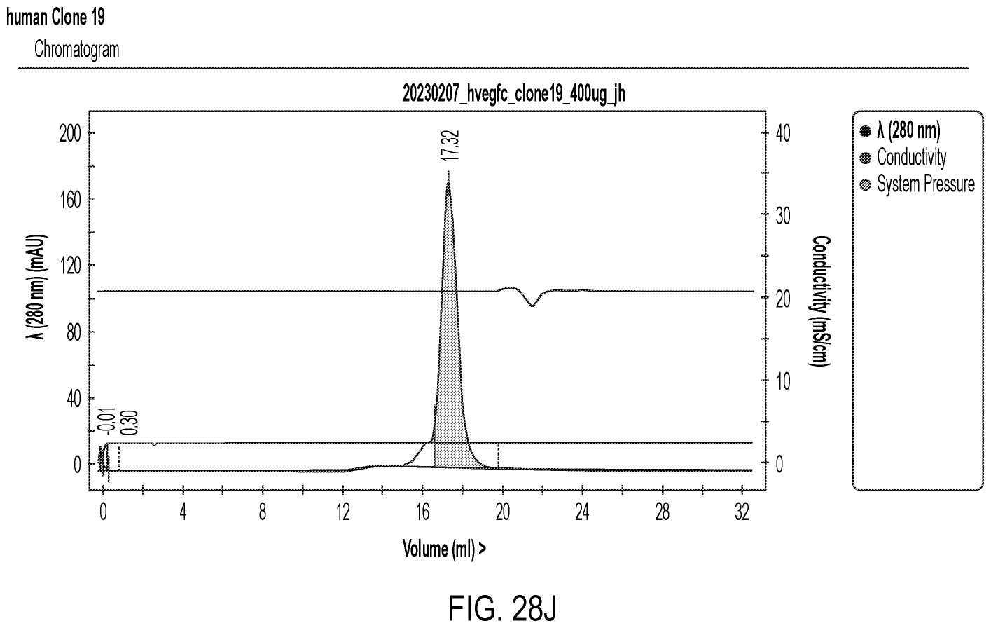

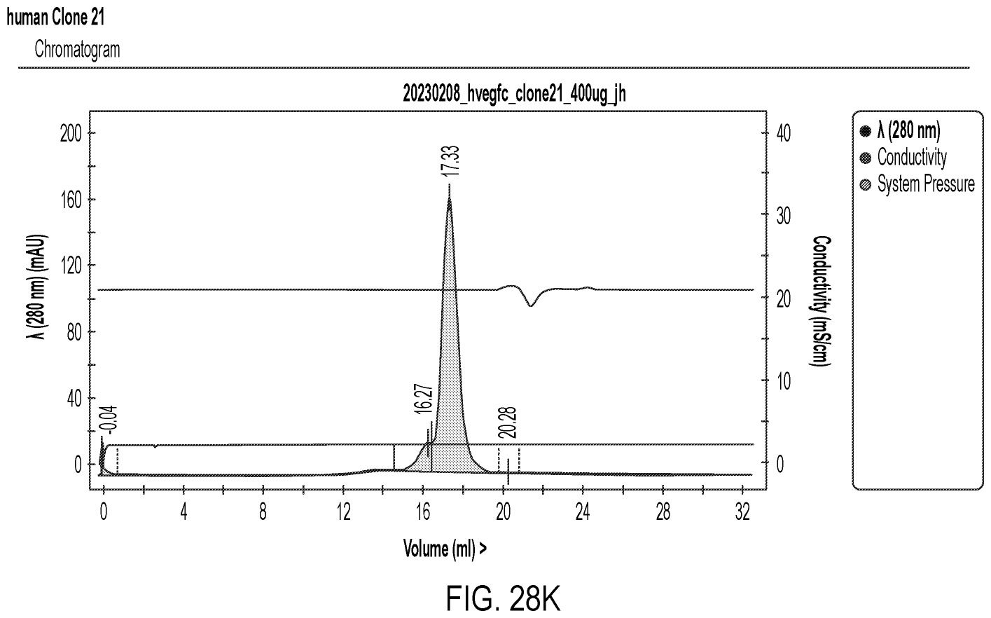

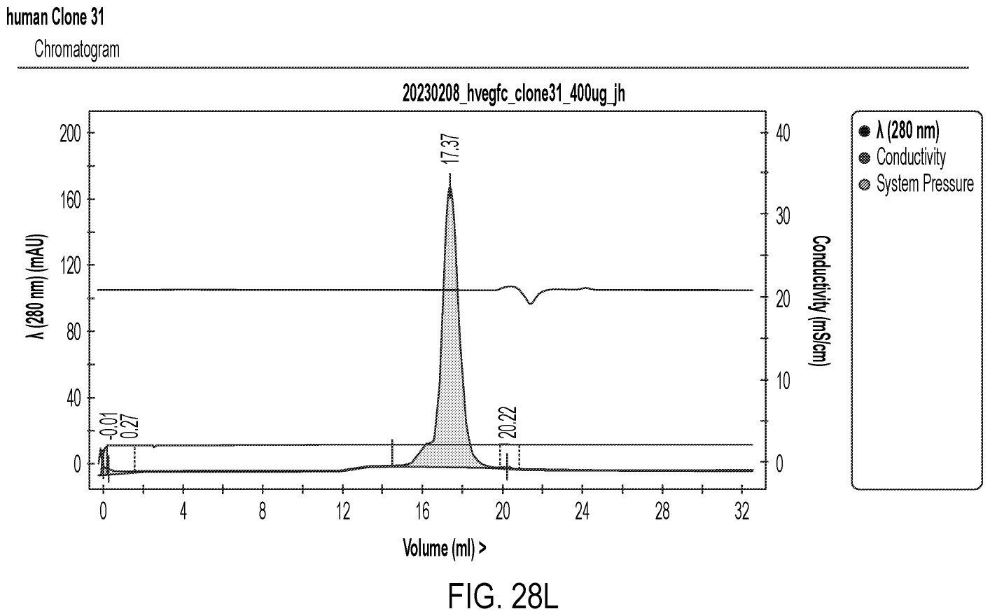

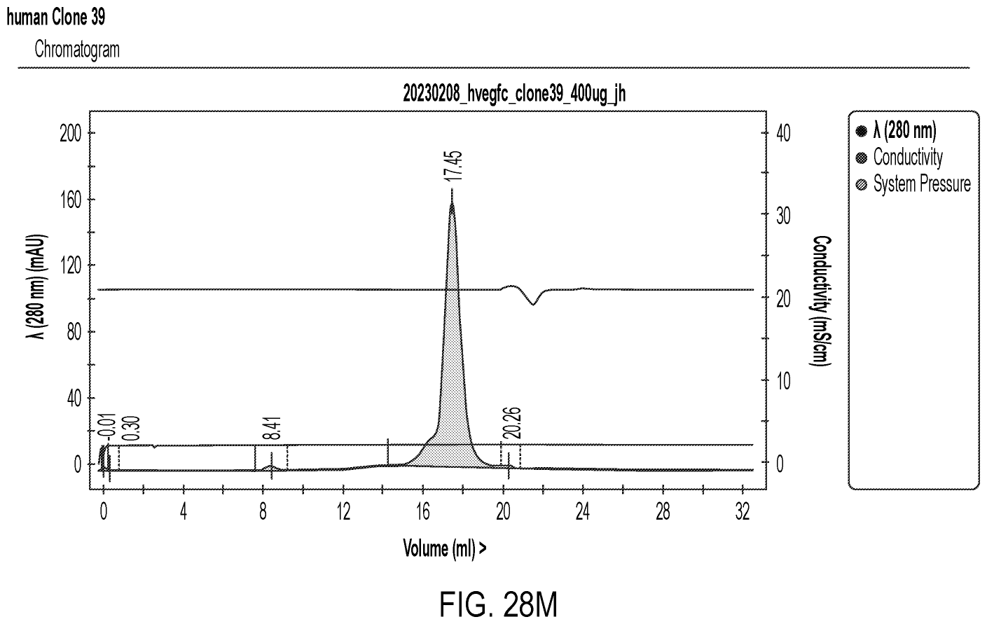

Human VEGFC muteins fused to human IgG1 Fc (N297Q) were transiently expressed in Expi293 cells using the Expifectamine 293 transfection kit (Thermo-Fisher Scientific) according to the manufacturer's instructions. Expression supernatants were then subjected to Ni-NTA chromatography and protein yield was quantified in milligrams per liter (Table 6). Quality was then assessed by size exclusion chromatography using a Superose 6 column (GE Healthcare) (FIGS. 28A-28M).

TABLE 6
Expression of human VEGFC muteins fused to human IgG1 Fc.

| Human Clone | Yield (mg/L) |
|---|---|
| C137A | 185.6 |
| 7 | 188.9 |
| 8 | 167.8 |
| 9 | 160.0 |
| 10 | 153.3 |
| 13 | 152.2 |
| 14 | 168.9 |
| 15 | 126.7 |
| 16 | 145.6 |
| 19 | 137.8 |
| 21 | 112.2 |
| 31 | 150.0 |
| 39 | 118.9 |

### Example 8. VEGFR3 Expression in Lymphatics is Likely the Source of Mediating the Decrease in Eye Pressure

VEGFR3fl/fl mice were bred with Cdh5aCRE, NESTINCRE and PROXICRE (all ERT2, tamoxifen inducible) mice to generate tamoxifen inducible, conditional knockout mice. In the Control settings, mice received just corn oil, in the other settings, mice received tamoxifen daily for 7 days. All mice were then given ocular hypertension with bead injection into the anterior chambers. After increases in intraocular pressure was confirmed (baseline), all mice were treated with RTI-Fc intravitreally and eye pressures were measured 7 days later.

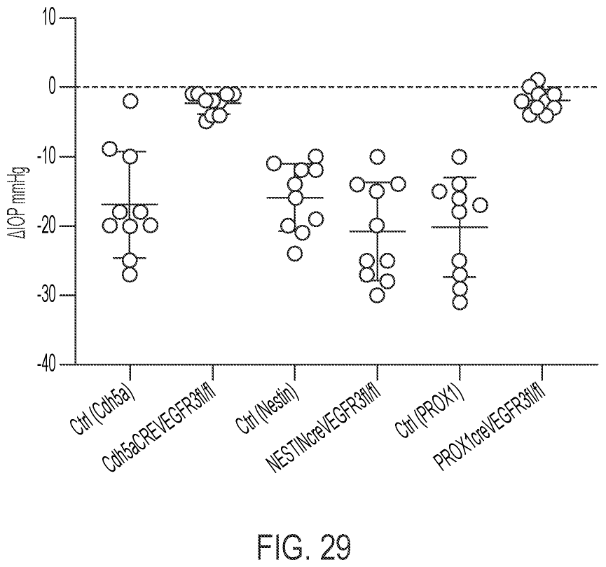

Data, shown in FIG. 29, demonstrated that VEGFR3 expression in prox1 and cdh5a positive cells result in decrease in eye pressure but not in nestin cells. Since prox1 is expressed in neurons and lymphatics, cdh5a in lymphatics and blood vessels, and nestin in neurons, this demonstrates that VEGFR3 expression in lymphatics is likely the source of mediating the decrease in eye pressure. Showing that RTI activity is specifically important in lymphatic endothelial cells.

### Example 9. Evaluation of VEGF-C Mutein Conjugates for Intraocular Pressure Drop

Wildtype mice were either treated with intravitreal LS-VEGF-C in the form of a monomer, with albumin conjugation or Fc conjugation and evaluated for intraocular pressure drops.

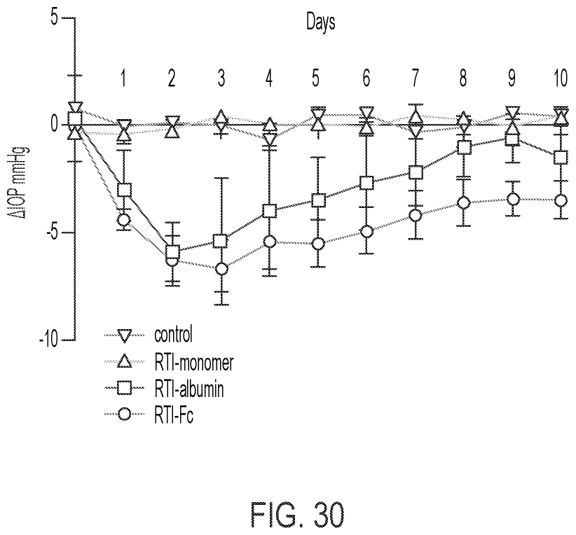

The monomer had almost no effect while the albumin conjugation and Fc conjugation had similar peak decreases in IOP with the Fc conjugate showing superiority overtime (FIG. 30).

### Discussion

The creation of a lymphatic-specific VEGF-C that provides a new molecular tool to study lymphatic biology is provided herein. Although lymphatic vasculature dysregulation is implicated in many disease processes, translation of these findings into clinic is limited by lack of methods to specifically stimulate lymphatics. Previous efforts identified muteins that lost VEGFR-2 binding, but also significantly lost binding towards VEGFR-3²⁴⁻²⁶. To address these clinical barriers, a library of VEGF-C muteins was created herein to generate LS-VEGF-C proteins that demonstrate picomolar binding to VEGFR-3 without binding VEGFR-2. By utilizing this new property, it was first highlighted herein the potential of this new molecule to treat various diseases known to have lymphatic dysfunction. By completely abrogating the angiogenic activity, LS-VEGF-C's activity could be further expanded into new disease spaces. The superior efficacy of LS-VEGF-C as a therapy for glaucoma was shown herein, a disease with clearly identifiable biophysical dysfunction that can benefit from lymphatic therapy-WT-VEGF-C is precluded as a possible candidate due to the dangers of angiogenesis in the eye. LS-VEGF-C not only demonstrated strong in vivo activity consistent with its binding properties, but also resulted in no angiogenesis or vascular permeability.

This is not the first time that lymphatic-based therapy was proposed for glaucoma. Trabecular meshwork and Schlemm's canal show signatures of lymphatic vasculature and have been identified as regions that can be stimulated by lymphatic signaling^{14,27,28}. This concept has also been applied to aqueous mapping in NHPs and humans²⁹. In addition, uveoscleral lymphatic pathways are also thought to be involved in aqueous humor drainage¹². As stated above, the focus on decreasing ocular pressure has been focused specifically in the anterior compartment, with all drugs and surgical procedures targeting components of the anterior eye. However, the pathology of glaucoma occurs in the posterior eye, with damage to the optic nerve being the primary sign of progression. It was decided herein that IOP decreasing focused in this compartment will provide a unique approach to complement current therapeutic strategies. As a therapeutic molecule, LS-VEGF-C demonstrated herein potent effects in decreasing IOP in two models of ocular hypertension which resulted in significant neuronal health preservation. Beyond this, it was demonstrated herein that stimulation of the optic nerve lymphatics allows for drainage of neurotoxic molecules from the eye that allows for neuroprotection.

Most importantly, LS-VEGF-C is not only a new pharmacological agent but a tool to answer questions regarding lymphatic biology. Previous reports have tried to specifically stimulate VEGFR-3 and concluded that without VEGFR-2 signaling, effects were not as potent³⁰. In contrast, LS-VEGF-C demonstrated stronger phenotypic differences compared to WT-VEGF-C in vivo. This is likely due to a combination of factors including 1) without VEGFR-2 binding, there is no VEGF-C being sequestered away from its lymphatic binding partner VEGFR-3 and 2) LS-VEGF-C retains picomolar concentration binding affinity to VEGFR-3. This makes our mutein not only an excellent candidate for clinical implications requiring lymphatic stimulation, but also a unique tool to decouple VEGFR-2 (angiogenic) and VEGFR-3 (lymphangiogenic) signaling. Its utility can be imagined for diseases with obvious indications such as lymphedema2 to recent discoveries in neurodegeneration^{22,23}, having potential to make significant impact in human health and disease. In summary, the present results from developing a VEGFR-3-specific ligand establish the therapeutic potential and versatility of lymphangiogenesis, even in immune-privileged spaces.

### REFERENCES

(1) Yang, Q.; Cho, K. S.; Chen, H.; Yu, D.; Wang, W. H.; Luo, G.; Pang, I. H.; Guo, W.; Chen, D. F. Microbead-Induced Ocular Hypertensive Mouse Model for Screening and Testing of Aqueous Production Suppressants for Glaucoma. Invest. Ophthalmol. Vis. Sci. 2012, 53 (7), 3733-3741. https://doi.org/10.1167/IOVS.12-9814.
(2) Szöke, D.; Kovács, G.; Kemecsei, É.; Bálint, L.; Szoták-Ajtay, K.; Aradi, P.; Styevkóné Dinnyés, A.; Mui, B. L.; Tam, Y. K.; Madden, T. D.; Karikó, K.; Kataru, R. P.; Hope, M. J.; Weissman, D.; Mehrara, B. J.; Pardi, N.; Jakus, Z. Nucleoside-Modified VEGFC MRNA Induces Organ-Specific Lymphatic Growth and Reverses Experimental Lymphedema. Nat. Commun. 2021 121 2021, 12 (1), 1-18. https://doi.org/10.1038/s41467-021-23546-6.
(3) Song, E.; Mao, T.; Dong, H.; Boisserand, L. S. B.; Antila, S.; Bosenberg, M.; Alitalo, K.; Thomas, J. L.; Iwasaki, A. VEGF-C-Driven Lymphatic Drainage Enables Immunosurveillance of Brain Tumours. Nat. 2020 5777792 2020, 577 (7792), 689-694. https://doi.org/10.1038/s41586-019-1912-x.
(4) Abhinand, C. S.; Raju, R.; Soumya, S. J.; Arya, P. S.; Sudhakaran, P. R. VEGF-A/VEGFR2 Signaling Network in Endothelial Cells Relevant to Angiogenesis. J. Cell Commun. Signal. 2016, 10 (4), 347. https://doi.org/10.1007/S12079-016-0352-8.
(5) Deng, Y.; Zhang, X.; Simons, M. Molecular Controls of Lymphatic VEGFR3 Signaling. Arterioscler. Thromb. Vasc. Biol. 2015, 35 (2), 421. https://doi.org/10.1161/ATVBAHA.114.304881.
(6) Petrova, T. V.; Koh, G. Y. Organ-Specific Lymphatic Vasculature: From Development to Pathophysiology. J. Exp. Med. 2018, 215 (1), 35-49. https://doi.org/10.1084/JEM.20171868.
(7) Karkkainen, M. J.; Haiko, P.; Sainio, K.; Partanen, J.; Taipale, J.; Petrova, T. V.; Jeltsch, M.; Jackson, D. G.; Talikka, M.; Rauvala, H.; Betsholtz, C.; Alitalo, K. Vascular Endothelial Growth Factor C Is Required for Sprouting of the First Lymphatic Vessels from Embryonic Veins. Nat. Immunol. 2004, 5 (1), 74-80. https://doi.org/10.1038/NI1013.
(8) Mäkinen, T.; Veikkola, T.; Mustjoki, S.; Karpanen, T.; Catimel, B.; Nice, E. C.; Wise, L.; Mercer, A.; Kowalski, H.; Kerjaschki, D.; Stacker, S. A.; Achen, M. G.; Alitalo, K. Isolated Lymphatic Endothelial Cells Transduce Growth, Survival and Migratory Signals via the VEGF-C/D Receptor VEGFR-3. EMBO J. 2001, 20 (17), 4762-4773. https://doi.org/10.1093/EMBOJ/20.17.4762.
(9) Szuba, A.; Skobe, M.; Karkkainen, M. J.; Shin, W. S.; Beynet, D. P.; Rockson, N. B.; Dakhil, N.; Spilman, S.; Goris, M. L.; Strauss, H. W.; Quertermous, T.; Alitalo, K.; Rockson, S. G. Therapeutic Lymphangiogenesis with Human Recombinant VEGF-C. FASEB J. 2002, 16 (14), 1985-1987. https://doi.org/10.1096/FJ.02-0401FJE.
(10) Wang, G.-Y; Zhong, S.-Z. A Model of Experimental Lymphedema in Rats' Limbs. Microsurgery 1985, 6 (4), 204-210. https://doi.org/10.1002/MICR.1920060404.
(11) Yücel, Y. H.; Cardinell, K.; Khattak, S.; Zhou, X.; Lapinski, M.; Cheng, F.; Gupta, N. Active Lymphatic Drainage From the Eye Measured by Noninvasive Photoacoustic Imaging of Near-Infrared Nanoparticles. Invest. Ophthalmol. Vis. Sci. 2018, 59 (7), 2699-2707. https://doi.org/10.1167/IOVS.17-22850.
(12) Yücel, Y. H.; Johnston, M. G.; Ly, T.; Patel, M.; Drake, B.; Gümüş, E.; Fraenkl, S. A.; Moore, S.; Tobbia, D.; Armstrong, D.; Horvath, E.; Gupta, N. Identification of Lymphatics in the Ciliary Body of the Human Eye: A Novel “Uveolymphatic” Outflow Pathway. Exp. Eye Res. 2009, 89 (5), 810-819. https://doi.org/10.1016/J.EXER.2009.08.010.
(13) Wang, X.; Lou, N.; Eberhardt, A.; Yang, Y.; Kusk, P.; Xu, Q.; Förstera, B.; Peng, S.; Shi, M.; Ladrón-De-Guevara, A.; Delle, C.; Sigurdsson, B.; Xavier, A. L. R.; Ertürk, A.; Libby, R. T.; Chen, L.; Thrane, A. S.; Nedergaard, M. An Ocular Glymphatic Clearance System Removes β-Amyloid from the Rodent Eye. Sci. Transl. Med. 2020, 12 (536), 3210. https://doi.org/10.1126/SCITRANSLMED.AAW3210/SUPPL_FILE/AAW3210_SM.PDF.
(14) Aspelund, A.; Tammela, T.; Antila, S.; Nurmi, H.; Leppänen, V. M.; Zarkada, G.; Stanczuk, L.; Francois, M.; Mäkinen, T.; Saharinen, P.; Immonen, I.; Alitalo, K. The Schlemm's Canal Is a VEGF-C/VEGFR-3-Responsive Lymphatic-like Vessel. J. Clin. Invest. 2014, 124 (9), 3975-3986. https://doi.org/10.1172/JCI75395.
(15) Pavlakovic, H.; Becker, J.; Albuquerque, R., Wilting, J.; Ambati, J. Soluble VEGFR-2: An Antilymphangiogenic Variant of VEGF Receptors. Ann. N. Y. Acad. Sci. 2010, 1207 (SUPPL.1), E7-E15. https://doi.org/10.1111/J.1749-6632.2010.05714.X.
(16) Subrizi, A.; del Amo, E. M.; Korzhikov-Vlakh, V.; Tennikova, T.; Ruponen, M.; Urtti, A. Design Principles of Ocular Drug Delivery Systems: Importance of Drug Payload, Release Rate, and Material Properties. Drug Discov. Today 2019, 24 (8), 1446-1457. https://doi.org/10.1016/J.DRUDIS.2019.02.001.
(17) Lommatzsch, C.; Rothaus, K.; Schopmeyer, L.; Feldmann, M.; Bauer, D.; Grisanti, S.; Heinz, C.; Kasper, M. Elevated Endothelin-1 Levels as Risk Factor for an Impaired Ocular Blood Flow Measured by OCT-A in Glaucoma. Sci. Reports 2022 121 2022, 12 (1), 1-12. https://doi.org/10.1038/s41598-022-15401-5.
(18) Sappington, R. M.; Carlson, B. J.; Crish, S. D.; Calkins, D. J. The Microbead Occlusion Model: A Paradigm for Induced Ocular Hypertension in Rats and Mice. Investig. Ophthalmol. Vis. Sci. 2010, 51 (1), 207-216. https://doi.org/10.1167/IOVS.09-3947.
(19) John, S. W.; Smith, R. S.; Savinova, O. V; Hawes, N. L.; Chang, B.; Turnbull, D.; Davisson, M.; Roderick, T. H.; Heckenlively, R. Essential Iris Atrophy, Pigment Dispersion, and Glaucoma in DBA/2J Mice. Invest. Ophthalmol. Vis. Sci. 1998, 39 (6), 951-962.
(20) Villanueva, J. R.; Esteban, J. M.; Villanueva, L. J. R. Retinal Cell Protection in Ocular Excitotoxicity Diseases. Possible Alternatives Offered by Microparticulate Drug Delivery Systems and Future Prospects. Pharmaceutics 2020, 12 (2). https://doi.org/10.3390/PHARMACEUTICS12020094.
(21) Marchesi, N.; Fahmideh, F.; Boschi, F.; Pascale, A., Barbieri, A. Ocular Neurodegenerative Diseases: Interconnection between Retina and Cortical Areas. Cells 2021, 10 (9). https://doi.org/10.3390/CELLS10092394.
(22) Da Mesquita, S.; Papadopoulos, Z.; Dykstra, T.; Brase, L.; Farias, F. G.; Wall, M.; Jiang, H.; Kodira, C. D.; de Lima, K. A.; Herz, J.; Louveau, A.; Goldman, D. H.; Salvador, A. F.; Onengut-Gumuscu, S.; Farber, E.; Dabhi, N.; Kennedy, T.; Milam, M. G.; Baker, W.; Smirnov, I.; Rich, S. S.; Benitez, B. A.; Karch, C. M.; Perrin, R. J.; Farlow, M.; Chhatwal, J. P.; Holtzman, D. M.; Cruchaga, C.; Harari, O.; Kipnis, J. Meningeal Lymphatics Modulate Microglial Activation and Immunotherapy in Alzheimer's Disease. Nature 2021, 593 (7858), 255. https://doi.org/10.1038/S41586-021-03489-0.
(23) Da Mesquita, S.; Louveau, A.; Vaccari, A.; Smirnov, I.; Cornelison, R. C.; Kingsmore, K. M.; Contarino, C.; Onengut-Gumuscu, S.; Farber, E.; Raper, D.; Viar, K. E.; Powell, R. D.; Baker, W.; Dabhi, N.; Bai, R.; Cao, R.; Hu, S.; Rich, S. S.; Munson, J. M.; Lopes, M. B.; Overall, C. C.; Acton, S. T.; Kipnis, J. Functional Aspects of Meningeal Lymphatics in Ageing and Alzheimer's Disease. Nat. 2018 5607717 2018, 560 (7717), 185-191. https://doi.org/10.1038/s41586-018-0368-8.
(24) Joukov, V.; Kumar, V.; Sorsa, T.; Arighi, E.; Weich, H.; Saksela, O.; Alitalo, K. A Recombinant Mutant Vascular Endothelial Growth Factor-C That Has Lost Vascular Endothelial Growth Factor Receptor-2 Binding, Activation, and Vascular Permeability Activities*. 1998. https://doi.org/10.1074/jbc.273.12.6599.
(25) Joukov, V.; Sorsa, T.; Kumar, V.; Jeltsch, M.; Claesson-Welsh, L.; Cao, Y.; Saksela, O.; Kalkkinen, N.; Alitalo, K. Proteolytic Processing Regulates Receptor Specificity and Activity of VEGF-C. EMBO J. 1997, 16 (13), 3898-3911. https://doi.org/10.1093/EMBOJ/16.13.3898.
(26) Jeltsch, M.; Karpanen, T.; Strandin, T.; Aho, K.; Lankinen, H.; Alitalo, K. Vascular Endothelial Growth Factor (VEGF)/VEGF-C Mosaic Molecules Reveal Specificity Determinants and Feature Novel Receptor Binding Patterns. J. Biol. Chem. 2006, 281 (17), 12187-12195. https://doi.org/10.1074/JBC.M511593200.
(27) Park, D. Y.; Lee, J.; Park, I.; Choi, D.; Lee, S.; Song, S.; Hwang, Y.; Hong, K. Y.; Nakaoka, Y.; Makinen, T.; Kim, P.; Alitalo, K.; Hong, Y. K.; Koh, G. Y. Lymphatic Regulator PROX1 Determines Schlemm's Canal Integrity and Identity. J. Clin. Invest. 2014, 124 (9), 3960-3974. https://doi.org/10.1172/JCI75392.
(28) Thomson, B. R.; Liu, P.; Onay, T.; Du, J.; Tompson, S. W.; Misener, S.; Purohit, R. R.; Young, T. L.; Jin, J.; Quaggin, S. E. Cellular Crosstalk Regulates the Aqueous Humor Outflow Pathway and Provides New Targets for Glaucoma Therapies. Nat. Commun. 2021, 12 (1). https://doi.org/10.1038/S41467-021-26346-0.
(29) Huang, A. S.; Li, M.; Yang, D.; Wang, H.; Wang, N.; Weinreb, R. N. Aqueous Angiography in Living Nonhuman Primates Shows Segmental, Pulsatile, and Dynamic Angiographic Aqueous Humor Outflow. Ophthalmology 2017, 124 (6), 793-803. https://doi.org/10.1016/J.OPHTHA.2017.01.030.
(30) Nilsson, I.; Bahram, F.; Li, X.; Gualandi, L.; Koch, S.; Jarvius, M.; Söderberg, O.; Anisimov, A.; Kholová, I.; Pytowski, B.; Baldwin, M.; Ylä-Herttuala, S.; Alitalo, K.; Kreuger, J.; Claesson-Welsh, L. VEGF Receptor 2/-3 Heterodimers Detected in Situ by Proximity Ligation on Angiogenic Sprouts. EMBO J. 2010, 29 (8), 1377-1388. https://doi.org/10.1038/EMBOJ.2010.30.
(31) Schlüter, A.; Aksan, B.; Diem, R.; Fairless, R.; Mauceri, D. VEGFD Protects Retinal Ganglion Cells and, Consequently, Capillaries against Excitotoxic Injury. Mol. Ther. Methods Clin. Dev. 2020, 17, 281. https://doi.org/10.1016/J.OMTM.2019.12.009.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

1. An isolated vascular endothelial growth factor C (VEGF-C) mutein protein or a functional fragment thereof, wherein the VEGF-C mutein protein or functional fragment thereof has a reduced or no ability to stimulate blood endothelial cell proliferation, as compared to a wild-type VEGF-C protein from the same species but preserves the ability to stimulate lymphatic endothelial cell proliferation.

2. An isolated vascular endothelial growth factor C (VEGF-C) mutein protein or a functional fragment thereof, wherein the VEGF-C mutein protein or functional fragment thereof (i) has a reduced binding affinity to vascular endothelial growth factor receptor-2 (VEGFR-2) as compared to a wild-type VEGF-C protein from the same species, (ii) has the ability to bind and generate signaling through vascular endothelial growth factor receptor-3 (VEGFR-3), and (iii) comprises an amino acid sequence that is at least 70% identical to the amino acid sequence of the wild-type VEGF-C protein from the same species.

3. The VEGF-C mutein protein or functional fragment thereof of claim 1 or claim 2, wherein the VEGF-C mutein protein or functional fragment thereof generates reduced or no signaling through VEGFR-2 as compared to the wild-type VEGF-C protein from the same species.

4. The VEGF-C mutein protein or functional fragment thereof of claim 3, wherein the VEGF-C mutein protein or functional fragment thereof does not generate signaling through VEGFR-2.

5. The VEGF-C mutein protein or functional fragment thereof of claim 3 or claim 4, wherein the signaling through VEGFR-2 is determined by measuring VEGFR-2-dependent AKT-phosphorylation and/or ERK-phosphorylation level in blood endothelial cells, by a wound healing assay (scratch assay), by a proliferation assay, or by an angiogenesis assay.

6. The VEGF-C mutein protein or functional fragment thereof of any one of claims 2-5, wherein the signaling through VEGFR-3 is determined by measuring VEGFR-3-dependent AKT-phosphorylation and/or ERK-phosphorylation level in lymphatic endothelial cells, by a wound healing assay, by a proliferation assay, or by an angiogenesis assay.

7. The VEGF-C mutein protein or functional fragment thereof of any one of claims 1-6, wherein the VEGF-C mutein protein or functional fragment thereof does not induce angiogenesis.

8. The VEGF-C mutein protein or functional fragment thereof of any of claims 1-7, wherein the VEGF-C mutein protein is a mutein of a wild-type VEGF-C protein comprising amino acids 111-211 of SEQ ID NO: 4, or a polypeptide defined by the corresponding positions within a wild-type VEGF-C protein of another species.

9. The VEGF-C mutein protein or functional fragment thereof of claim 8, wherein the wild-type VEGF-C protein comprises amino acids 111-211 of SEQ ID NO: 4.

10. The VEGF-C mutein protein or functional fragment thereof of any one of claims 1-9, wherein the VEGF-C mutein protein or functional fragment thereof comprises one or more mutations selected from mutations at residues T112, L115, D119, Q126, T144, N145, K149, N163, S164, E165, I184, V186, L188, and P192, wherein the positions of said residues are defined in relation to SEQ ID NO: 4, or mutations at the corresponding residues within the wild-type VEGF-C protein of another species.

11. The VEGF-C mutein protein or functional fragment thereof of any one of claims 1-10, wherein the VEGF-C mutein protein or functional fragment thereof further comprises a mutation at residue C133, wherein the position of said residue is defined in relation to SEQ ID NO: 4, or a mutation at the corresponding residue within the wild-type VEGF-C protein of another species.

12. The VEGF-C mutein protein or functional fragment thereof of claim 11, wherein the mutation at residue C133 is C133A mutation.

13. The VEGF-C mutein protein or functional fragment thereof of any of claims 1-7, wherein the VEGF-C mutein protein is a mutein of a wild-type VEGF-C protein comprising amino acids 115-215 of SEQ ID NO: 1, or a polypeptide defined by the corresponding positions within the wild-type VEGF-C protein of another species.

14. The VEGF-C mutein protein or functional fragment thereof of claim 13, wherein the wild-type VEGF-C protein comprises amino acids 115-215 of SEQ ID NO: 1.

15. The VEGF-C mutein protein or functional fragment thereof of any one of claims 1-7 and 13-14, wherein the VEGF-C mutein protein or functional fragment thereof comprises one or more mutations selected from mutations at residues T116, L119, D123, Q130, T148, N149, K153, N167, S168, E169, I188, V190, L192, and P196, wherein the positions of said residues are defined in relation to SEQ ID NO: 1.

16. The VEGF-C mutein protein or functional fragment thereof of any one of claims 1-7 and 13-15, wherein the VEGF-C mutein protein or functional fragment thereof further comprises a mutation at residue C137, wherein the position of said residue is defined in relation to SEQ ID NO: 1, or a mutation at the corresponding residue within the wild-type VEGF-C protein of another species.

17. The VEGF-C mutein protein or functional fragment thereof of claim 16, wherein the mutation at residue C137 is C137A mutation.

18. The VEGF-C mutein protein or functional fragment thereof of any one of claims 15-17, wherein the mutation at residue L119 is L119E mutation, or L119M mutation; the mutation at residue D123 is D123N mutation; the mutation at residue Q130 is Q130K mutation; the mutation at residue N167 is N167R mutation, N167I mutation, N167Q mutation, or N167H mutation; the mutation at residue S168 is S168G mutation, or S168R mutation; the mutation at residue V190 is V190T mutation; and/or the mutation at residue L192 is L192I mutation.

19. The VEGF-C mutein protein or functional fragment thereof of any one of claims 15-18, wherein the VEGF-C mutein protein or functional fragment thereof comprises one or more mutations selected from mutations at residues N167, S168, and/or L192.

20. The VEGF-C mutein protein or functional fragment thereof of claim 19, wherein the mutation at residue N167 is N167I mutation, N167Q mutation, or N167H mutation; the mutation at residue S168 is S168G mutation, or S168R mutation; and/or the mutation at residue L192 is L192I mutation.

21. The VEGF-C mutein protein or functional fragment thereof of claim 20, wherein the VEGF-C mutein protein or functional fragment thereof comprises N167Q mutation.

22. The VEGF-C mutein protein of claim 21, wherein the VEGF-C mutein protein comprises the amino acid sequence of SEQ ID NO: 160 or SEQ ID NO: 56.

23. The VEGF-C mutein protein of claim 22, wherein the VEGF-C mutein protein consists of the amino acid sequence of SEQ ID NO: 160 or SEQ ID NO: 56.

24. The VEGF-C mutein protein or functional fragment thereof of claim 20, wherein the VEGF-C mutein protein or functional fragment thereof comprises N167Q mutation and S168G mutation.

25. The VEGF-C mutein protein of claim 24, wherein the VEGF-C mutein protein comprises the amino acid sequence of SEQ ID NO: 161 or SEQ ID NO: 57.

26. The VEGF-C mutein protein of claim 25, wherein the VEGF-C mutein protein consists of the amino acid sequence of SEQ ID NO: 161 or SEQ ID NO: 57.

27. The VEGF-C mutein protein or functional fragment thereof of claim 20, wherein the VEGF-C mutein protein or functional fragment thereof comprises N167Q mutation and L192I mutation.

28. The VEGF-C mutein protein of claim 27, wherein the VEGF-C mutein protein comprises the amino acid sequence of SEQ ID NO: 162 or SEQ ID NO: 58.

29. The VEGF-C mutein protein of claim 28, wherein the VEGF-C mutein protein consists of the amino acid sequence of SEQ ID NO: 162 or SEQ ID NO: 58.

30. The VEGF-C mutein protein or functional fragment thereof of claim 20, wherein the VEGF-C mutein protein or functional fragment thereof comprises N167Q mutation, S168G mutation, and L192I mutation.

31. The VEGF-C mutein protein of claim 30, wherein the VEGF-C mutein protein comprises the amino acid sequence of SEQ ID NO: 163 or SEQ ID NO: 59.

32. The VEGF-C mutein protein of claim 31, wherein the VEGF-C mutein protein consists of the amino acid sequence of SEQ ID NO: 163 or SEQ ID NO: 59.

33. The VEGF-C mutein protein or functional fragment thereof of claim 20, wherein the VEGF-C mutein protein or functional fragment thereof comprises N167I mutation.

34. The VEGF-C mutein protein of claim 33, wherein the VEGF-C mutein protein comprises the amino acid sequence of SEQ ID NO: 166 or SEQ ID NO: 62.

35. The VEGF-C mutein protein of claim 34, wherein the VEGF-C mutein protein consists of the amino acid sequence of SEQ ID NO: 166 or SEQ ID NO: 62.

36. The VEGF-C mutein protein or functional fragment thereof of claim 20, wherein the VEGF-C mutein protein or functional fragment thereof comprises N167I mutation and S168G mutation.

37. The VEGF-C mutein protein of claim 36, wherein the VEGF-C mutein protein comprises the amino acid sequence of SEQ ID NO: 167 or SEQ ID NO: 63.

38. The VEGF-C mutein protein of claim 37, wherein the VEGF-C mutein protein consists of the amino acid sequence of SEQ ID NO: 167 or SEQ ID NO: 63.

39. The VEGF-C mutein protein or functional fragment thereof of claim 20, wherein the VEGF-C mutein protein or functional fragment thereof comprises N167I mutation and L192I mutation.

40. The VEGF-C mutein protein of claim 39, wherein the VEGF-C mutein protein comprises the amino acid sequence of SEQ ID NO: 168 or SEQ ID NO: 64.

41. The VEGF-C mutein protein of claim 40, wherein the VEGF-C mutein protein consists of the amino acid sequence of SEQ ID NO: 168 or SEQ ID NO: 64.

42. The VEGF-C mutein protein or functional fragment thereof of claim 20, wherein the VEGF-C mutein protein or functional fragment thereof comprises N167I mutation, S168G mutation, and L192I mutation.

43. The VEGF-C mutein protein of claim 42, wherein the VEGF-C mutein protein comprises the amino acid sequence of SEQ ID NO: 169 or SEQ ID NO: 65.

44. The VEGF-C mutein protein of claim 43, wherein the VEGF-C mutein protein consists of the amino acid sequence of SEQ ID NO: 169 or SEQ ID NO: 65.

45. The VEGF-C mutein protein or functional fragment thereof of claim 20, wherein the VEGF-C mutein protein or functional fragment thereof comprises S168G mutation.

46. The VEGF-C mutein protein of claim 45, wherein the VEGF-C mutein protein comprises the amino acid sequence of SEQ ID NO: 172 or SEQ ID NO: 68.

47. The VEGF-C mutein protein of claim 46, wherein the VEGF-C mutein protein consists of the amino acid sequence of SEQ ID NO: 172 or SEQ ID NO: 68.

48. The VEGF-C mutein protein or functional fragment thereof of claim 20, wherein the VEGF-C mutein protein or functional fragment thereof comprises S168G mutation and L192I mutation.

49. The VEGF-C mutein protein of claim 48, wherein the VEGF-C mutein protein comprises the amino acid sequence of SEQ ID NO: 174 or SEQ ID NO: 70.

50. The VEGF-C mutein protein of claim 49, wherein the VEGF-C mutein protein consists of the amino acid sequence of SEQ ID NO: 174 or SEQ ID NO: 70.

51. The VEGF-C mutein protein or functional fragment thereof of claim 20, wherein the VEGF-C mutein protein or functional fragment thereof comprises N167H mutation.

52. The VEGF-C mutein protein of claim 51 wherein the VEGF-C mutein protein comprises the amino acid sequence of SEQ ID NO: 184 or SEQ ID NO: 80.

53. The VEGF-C mutein protein of claim 52, wherein the VEGF-C mutein protein consists of the amino acid sequence of SEQ ID NO: 184 or SEQ ID NO: 80.

54. The VEGF-C mutein protein or functional fragment thereof of claim 20, wherein the VEGF-C mutein protein or functional fragment thereof comprises N167I mutation and S168R mutation.

55. The VEGF-C mutein protein of claim 54, wherein the VEGF-C mutein protein comprises the amino acid sequence of SEQ ID NO: 192 or SEQ ID NO: 88.

56. The VEGF-C mutein protein of claim 55, wherein the VEGF-C mutein protein consists of the amino acid sequence of SEQ ID NO: 192 or SEQ ID NO: 88.

57. A fusion protein or conjugate comprising the VEGF-C mutein protein or functional fragment thereof of any of claims 1-56, wherein the mutein protein or functional fragment thereof, is fused and/or conjugated to one of more heterologous moieties.

58. The fusion protein or conjugate of claim 57, wherein the one of more heterologous moieties are selected from an immunoglobulin or a functional fragment thereof, an albumin or a functional fragment thereof, an albumin-binding antibody or a functional fragment thereof, and a polyethylene glycol (PEG) polymer.

59. The fusion protein or conjugate of claim 58, wherein the immunoglobulin or functional fragment thereof comprises an IgG Fc domain.

60. The fusion protein of claim 59, wherein the IgG Fc domain is modified to reduce a Fc effector function.

61. The fusion protein of claim 60, wherein the IgG Fc domain comprises a mutation at residue N297.

62. The fusion protein of claim 61, wherein the mutation at residue N297 is selected from N297Q, N297A and N297G.

63. An isolated polynucleotide molecule encoding the VEGF-C mutein protein or functional fragment thereof of any one of claims 1-56 or the fusion protein or conjugate of any one of claims 57-62.

64. The polynucleotide molecule of claim 63, wherein the polynucleotide molecule comprises the nucleotide sequence of SEQ ID NO: 210 or SEQ ID NO: 108.

65. The polynucleotide molecule of claim 63, wherein the polynucleotide molecule comprises the nucleotide sequence of SEQ ID NO: 211 or SEQ ID NO: 109.

66. The polynucleotide molecule of claim 63, wherein the polynucleotide molecule comprises the nucleotide sequence of SEQ ID NO: 212 or SEQ ID NO: 110.

67. The polynucleotide molecule of claim 63, wherein the polynucleotide molecule comprises the nucleotide sequence of SEQ ID NO: 213 or SEQ ID NO: 111.

68. The polynucleotide molecule of claim 63, wherein the polynucleotide molecule comprises the nucleotide sequence of SEQ ID NO: 216 or SEQ ID NO: 114.

69. The polynucleotide molecule of claim 63, wherein the polynucleotide molecule comprises the nucleotide sequence of SEQ ID NO: 217 or SEQ ID NO: 115.

70. The polynucleotide molecule of claim 63, wherein the polynucleotide molecule comprises the nucleotide sequence of SEQ ID NO: 218 or SEQ ID NO: 116.

71. The polynucleotide molecule of claim 63, wherein the polynucleotide molecule comprises the nucleotide sequence of SEQ ID NO: 219 or SEQ ID NO: 117.

72. The polynucleotide molecule of claim 63, wherein the polynucleotide molecule comprises the nucleotide sequence of SEQ ID NO: 222 or SEQ ID NO: 120.

73. The polynucleotide molecule of claim 63, wherein the polynucleotide molecule comprises the nucleotide sequence of SEQ ID NO: 224 or SEQ ID NO: 122.

74. The polynucleotide molecule of claim 63, wherein the polynucleotide molecule comprises the nucleotide sequence of SEQ ID NO: 234 or SEQ ID NO: 132.

75. The polynucleotide molecule of claim 63, wherein the polynucleotide molecule comprises the nucleotide sequence of SEQ ID NO: 242 or SEQ ID NO: 140.

76. The polynucleotide molecule of any one of claims 63-75, wherein the polynucleotide molecule comprises a nucleotide sequence encoding the VEGF-C mutein protein or functional fragment thereof which is operably linked to a promoter.

77. The polynucleotide molecule of any one of claims 63-75, wherein the polynucleotide molecule is an mRNA.

78. The polynucleotide molecule of any one of claims 63-77, wherein the polynucleotide molecule comprises one or more nucleotide modifications.

79. The polynucleotide molecule of claim 78, wherein the one or more nucleotide modifications are a 5′cap, a 5-methylcytosine, or a pseudo-uridine.

80. A vector comprising the polynucleotide molecule of any one of claims 63-79.

81. The vector of claim 80, wherein the vector is a viral vector.

82. The vector of claim 81, wherein the viral vector is derived from a herpes virus, a cytomegalovirus, a poliovirus, an alphavirus, a vaccinia virus, a rabies virus, an adeno-associated virus (AAV), a retrovirus, a lentivirus, or an adenovirus.

83. A particle comprising the polynucleotide molecule of any one of claims 63-79.

84. The particle of claim 83, wherein the particle is a nanoparticle, lipid particle, microparticle, lipid nanoparticle, polymer particle, or virus-like particle (VLP).

85. A host cell comprising the polynucleotide of any one of claims 63-79 or the vector of any one of claims 80-82.

86. A method of producing a VEGF-C mutein protein or functional fragment thereof, or fusion protein thereof, comprising culturing the host cell of claim 85 under conditions at which the VEGF-C mutein protein or functional fragment thereof, or fusion protein thereof is expressed.

87. A VEGF-C mutein protein or functional fragment thereof, or fusion protein thereof, produced by the method of claim 86.

88. A kit comprising the VEGF-C mutein protein or functional fragment thereof of any one of claims 1-56 or the fusion protein or conjugate of any one of claims 57-62, and optionally instructions for use.

89. A kit comprising the polynucleotide of any of claims 63-79 or the vector of any one of claims 80-82, or the particle of claim 83 or claim 84, and optionally instructions for use.

90. A pharmaceutical composition comprising a VEGF-C mutein protein or functional fragment thereof of any of claims 1-56 or the fusion protein or conjugate of any one of claims 57-62, or the polynucleotide molecule of any one of claims 63-79, or the vector of any one of claims 80-82, or the particle of claim 83 or claim 84, and a pharmaceutically acceptable carrier or diluent.

91. The pharmaceutical composition of claim 90, wherein the composition comprises mRNA encoding the VEGF-C mutein protein or functional fragment thereof, or fusion protein thereof as mRNA-nanoparticle formulation.

92. The pharmaceutical composition of claim 90 or claim 91, further comprising an immunotherapeutic agent.

93. The pharmaceutical composition of claim 92, wherein the immunotherapeutic agent is an immune checkpoint inhibitor.

94. The pharmaceutical composition of claim 93, wherein the immune checkpoint inhibitor targets PD-1, PD-L1, CTLA-4, TIGIT, TIM-3, LAG-3, BTLA, GITR, 4-1BB, or Ox-40.

95. The pharmaceutical composition of claim 94, wherein the immune checkpoint inhibitor is an anti-PD-1 antibody, an anti-PD-L1 antibody, an anti-CTLA-4 antibody, an anti-TIGIT antibody, an anti-TIM-3 antibody, an anti-LAG-3 antibody, an anti-BLTA antibody, an anti-GITR antibody, an anti-4-IBB antibody, or an anti-Ox-40 antibody.

96. The pharmaceutical composition of any one of claims 90-95, wherein the composition is formulated for intrathecal administration.

97. The pharmaceutical composition of any one of claims 90-95, wherein the composition is formulated for intratumoral administration.

98. The pharmaceutical composition of any one of claims 90-95, wherein the composition is formulated for systemic administration.

99. The pharmaceutical composition of any one of claims 90-95, wherein the composition is formulated for intracisternal administration.

100. The pharmaceutical composition of any of claims 90-95, wherein the composition is formulated for eye-drop administration.

101. The pharmaceutical composition of any of claims 90-95, wherein the composition is formulated for intraocular administration.

102. A method of inducing lymphangiogenesis in a subject in need thereof, the method comprising administering to the subject an effective amount of the VEGF-C mutein protein or functional fragment thereof of any of claims 1-56 or the fusion protein or conjugate of any one of claims 57-62, or the polynucleotide molecule of any one of claims 63-79, or the vector of any one of claims 80-82, or the particle of claim 83 or claim 84, or the pharmaceutical composition of any one of claims 90-101.

103. The method of claim 102, wherein the administration of said VEGF-C mutein protein or functional fragment thereof, fusion protein, conjugate, polynucleotide molecule, vector, particle, or pharmaceutical composition, does not cause one or more side effects associated with administration of a wild-type VEGF-C protein, or corresponding fusion protein, conjugate, polynucleotide molecule, vector, particle, or pharmaceutical composition.

104. The method of claim 103, wherein the one or more side effects are angiogenesis and/or increased intraocular pressure (IOP).

105. The method of any one of claims 102-104, wherein the VEGF-C mutein protein or functional fragment thereof, fusion protein, conjugate, polynucleotide molecule, vector, particle, or pharmaceutical composition is administered intrathecally, intraocularly, intratumorally, intracisternally, intravitreally, via eye drops, subcutaneously, intradermally, via inhalation, via long-dwelling catheter, orally, topically, or systemically

106. The method of any one of claims 102-104, wherein the VEGF-C mutein protein or functional fragment thereof, fusion protein, conjugate, polynucleotide molecule, vector, particle, or pharmaceutical composition is administered to the cisterna magna or directly into the lymphatic system.

107. The method of any one of claims 102-107, wherein the subject has a disease or condition selected from cancer, coronary vessel function, osmoregulation, heart ischemia, restenosis, fibrosis, colitis, chronic liver disease, polycystic kidney disease, diseases or conditions associated with lymph node transplant, Alzheimer's disease, Parkinson's disease, stroke, cerebral ischemia, wound healing, lymphedema, Hennekam syndrome, Milroy's disease, Turner syndrome, age related macular degeneration, glaucoma, central serous chorioretinopathy, diabetic retinopathy, macular edema and retinal edema.

108. The method of claim 107, wherein the diseases or conditions associated with lymph node transplant are breast cancer associated lymphedema, idiopathic lymphedema, and/or heart failure associated lymphedema.

109. A method of treating a disease or condition in a subject in need thereof, the method comprising administering to the subject an effective amount of a VEGF-C mutein protein or functional fragment thereof of any of claims 1-56, or the fusion protein or conjugate of any one of claims 57-62, or the polynucleotide molecule of any one of claims 63-79, or the vector of any one of claims 80-82, or the particle of claim 83 or claim 84, or the pharmaceutical composition of any one of claims 90-101.

110. The method of claim 109, wherein the disease or condition is cancer, coronary vessel function, osmoregulation, heart ischemia, restenosis, fibrosis, colitis, chronic liver disease, polycystic kidney disease, diseases or conditions associated with lymph node transplant, Alzheimer's disease, Parkinson's disease, stroke, cerebral ischemia, wound healing, lymphedema, Hennekam syndrome, Milroy's disease, Turner syndrome, age related macular degeneration, glaucoma, central serous chorioretinopathy, diabetic retinopathy, macular edema, and retinal edema.

111. The method of claim 110, wherein the cancer is melanoma, lung cancer, breast cancer, stomach cancer, esophageal cancer, ovarian cancer, uterine cancer, cervical cancer, head and neck squamous cell carcinoma, thyroid cancer, liquid cancer, kidney cancers, urothelial bladder cancers, prostate cancer, pheochromocytoma, cholangiocarcinoma, liver hepatocellular carcinoma, pancreatic ductal adenocarcinoma, thymoma, sarcoma, mesothelioma, testicular cancer, or colorectal cancer.

112. The method of claim 110, wherein the cancer is in the brain or the central nervous system of the subject.

113. The method of claim 110, wherein the cancer is selected from glioma, ependymoma, subependymoma, primitive neuroectodermal tumor, ganglioglioma, Schwannoma, germinoma, craniopharyngioma, meningioma, CNS lymphoma, pineal tumor, retinoblastoma, uveal melanoma, and rhabdoid tumor.

114. The method of any one of claims 109-113, further comprising administering an immunotherapeutic agent.

115. The method of claim 114, wherein the immunotherapeutic agent is an immune checkpoint inhibitor.

116. The method of claim 115, wherein the immune checkpoint inhibitor targets PD-1, PD-L1, CTLA-4, TIGIT, TIM-3, LAG-3, BTLA, GITR, 4-1BB, or Ox-40.

117. The method of claim 116, wherein the immune checkpoint inhibitor is an anti-PD-1 antibody, an anti-PD-L1 antibody, an anti-CTLA-4 antibody, an anti-TIGIT antibody, an anti-TIM-3 antibody, an anti-LAG-3 antibody, an anti-BLTA antibody, an anti-GITR antibody, an anti-4-IBB antibody, or an anti-Ox-40 antibody.

118. The method of any one of claims 109-117, wherein the VEGF-C mutein protein or functional fragment thereof, fusion protein, conjugate, polynucleotide molecule, vector, particle, or pharmaceutical composition is administered intrathecally, intraocularly, intratumorally, intracisternally, intravitreally, via eye drops, subcutaneously, intradermally, via inhalation, via long-dwelling catheter, orally, topically, or systemically.

119. The method of any one of claims 109-117, wherein the VEGF-C mutein protein or functional fragment thereof, fusion protein, conjugate, polynucleotide molecule, vector, particle, or pharmaceutical composition is administered to the cisterna magna or directly into the lymphatic system.

120. The method of any one of claims 109-119, wherein the disease is cancer, and wherein the method further comprises administering an additional anti-cancer treatment to the subject.

121. The method of claim 120, wherein the additional anti-cancer treatment is selected from surgery, radiation therapy, administration of a chemotherapeutic agent, an immunotherapy, and any combinations thereof.

122. A method for modulating intraocular pressure in a subject in need thereof comprising administering to the subject an effective amount of the VEGF-C mutein protein or functional fragment thereof of any of claims 1-56, or the fusion protein or conjugate of any one of claims 57-62, or the polynucleotide molecule of any one of claims 63-79, or the vector of any one of claims 80-82, or the particle of claim 83 or claim 84, or the pharmaceutical composition of any one of claims 90-101, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above.

123. The method of claim 122, wherein the VEGF-C mutein protein or the corresponding wild-type VEGF-C protein, or functional fragment thereof, fusion protein, conjugate, polynucleotide molecule, vector, particle, or pharmaceutical composition, or the corresponding wild-type VEGF-C protein or functional fragment thereof, or the fusion protein or conjugate thereof, or the polynucleotide molecule encoding said wild-type VEGF-C protein or functional fragment thereof, or the vector or particle comprising said polynucleotide molecule, or the pharmaceutical composition comprising any of the above is administered to the posterior eye.

124. The method of any one of claims 122-123, wherein the administration is intraocular.

125. The method of claim 124, wherein the intraocular administration is intravitreal, via eye drops, or subretinal.

126. A method for removing unwanted fluid in an eye of a subject in need thereof comprising administering to the subject an effective amount of the VEGF-C mutein protein or functional fragment thereof of any of claims 1-56, or the fusion protein or conjugate of any one of claims 57-62, or the polynucleotide molecule of any one of claims 63-79, or the vector of any one of claims 80-82, or the particle of claim 83 or claim 84, or the pharmaceutical composition of any one of claims 90-101, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above.

127. The method of claim 126, wherein the unwanted fluid is optic nerve, retinal, subretinal, choroidal, or suprachoroidal fluid.

128. The method of any one of claims 122-127, wherein the subject has glaucoma, macular edema, central serous chorioretinopathy, retinal edema, papilledema, macular degeneration, or diabetic retinopathy.

129. The method of any one of claims 124-126, wherein the administration is intraocular.

130. The method of claim 127, wherein the intraocular administration is intravitreal, via eye drops, or subretinal.

131. A method for providing neuroprotection in a subject in need thereof comprising administering to the subject an effective amount of the VEGF-C mutein protein or functional fragment thereof of any of claims 1-56, or the fusion protein or conjugate of any one of claims 57-62, or the polynucleotide molecule of any one of claims 63-79, or the vector of any one of claims 80-82, or the particle of claim 83 or claim 84, or the pharmaceutical composition of any one of claims 90-101, or a corresponding wild-type VEGF-C protein or functional fragment thereof, or a fusion protein or conjugate thereof, or a polynucleotide molecule encoding said wild-type VEGF-C protein or functional fragment thereof, or a vector or particle comprising said polynucleotide molecule, or a pharmaceutical composition comprising any of the above.

132. A vaccine comprising the VEGF-C mutein protein or functional fragment thereof of any of claims 1-56, or the fusion protein or conjugate of any one of claims 57-62, or the polynucleotide molecule of any one of claims 63-79, or the vector of any one of claims 80-82, or the particle of claim 83 or claim 84, or the pharmaceutical composition of any one of claims 90-101.

133. A method inducing an immune response in a subject in need thereof in a subject in need thereof, the method comprising administering to the subject an effective amount of the vaccine of claim 132.

134. The method of any one of claims 102-133, wherein the subject is a human.

135. A method of generating a library of VEGF-C mutein proteins having selective binding to VEGFR-3, wherein amino acid residues comprising the shared binding interface on VEGF-C protein which binds both VEGFR-3 and VEGFR-2 are diversified to other amino acids via mutagenesis of the corresponding nucleic acid sequence.

136. A yeast cell library for selection of VEGF-C mutein proteins, comprising a plurality of yeast cells comprising a cell wall peptide anchor sequence, a linker peptide, and the VEGF-C mutein protein sequence.
